SEQUENCE LISTING (185 sequences)

SEQ ID NO: 1 - protein, 1210 aa, Homo sapiens [sequence omitted]

SEQ ID NO: 2 - DNA, 1869 bp, Homo sapiens
  misc_feature 1..1869: note = Wild type human EGFR ectodomain cDNA
  source 1..1869: mol_type = genomic DNA
ctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgcc

SEQ ID NO: 3 - protein, 621 aa, Homo sapiens [sequence omitted]

SEQ ID NO: 4 - DNA, 996 bp, Homo sapiens
  misc_feature 1..996: note = Human EGFRvIII ectodomain cDNA sequence (nucleotides 1-996)
  source 1..996: mol_type = genomic DNA
ctggaagagaagaaaggcaactacgtcgtgaccgaccacggcagctgtgtgcgggcttgtggcgccgatagctacgagatggaagaggacggcgtgcggaagtgcaagaagtgcgagggcccctgccggaaagtgtgcaacggcatcggcatcggagagttcaaggacagcctgagcatcaacgccaccaacatcaagcacttcaagaactgcaccagcatcagcggcgacctgcacatcctgcccgtggcctttagaggcgacagcttcacccacacccccccactggacccccaggaactggacatcctgaaaaccgtgaaagagatcaccggctttctgctgattcaggcctggcccgagaaccggacagacctgcacgccttcgagaacctggaaatcatccggggcaggaccaagcagcacggccagttttctctggccgtggtgtccctgaacatcaccagcctgggcctgcggagcctgaaagaaatcagcgacggcgacgtgatcatctccggcaacaagaacctgtgctacgccaacaccatcaactggaagaagctgttcggcacctccggccagaaaacaaagatcatcagcaaccggggcgagaacagctgcaaggccacaggacaagtgtgccacgccctgtgtagccctgagggctgttggggacccgagcccagagattgcgtgtcctgcagaaacgtgtcccggggcagagaatgcgtggacaagtgcaacctgctggaaggcgagccccgcgagttcgtggaaaacagcgagtgcatccagtgccaccccgagtgtctgccccaggccatgaacattacctgcaccggcagaggccccgacaactgtatccagtgcgcccactacatcgacggcccccactgcgtgaaaacctgtcctgctggcgtgatgggagagaacaacaccctcgtgtggaagtacgccgacgccggccatgtgtgccacctgtgtcaccccaat

SEQ ID NO: 5 - protein, 332 aa, Homo sapiens
  REGION 1..332: note = Human EGFRvIII ectodomain (amino acids 1-332)
LEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPN

SEQ ID NO: 6 - protein, 23 aa, Homo sapiens
  REGION 1..23: note = Human EGFRvIII amino acid residues 15 to 37
SCVRACGADSYEMEEDGVRKCKK

SEQ ID NO: 7 - protein, 112 aa, synthetic construct
  REGION 1..112: note = 5G6 light chain variable region
DVVMTQTPLTLSVTIGQPASISCKSSQSLLDSDGKTYLNWLLQRPGQSPKRLIYLASKLDSGVPDRFTGSGSGTDFTLKISRVEAEDLGVYYCWQATHFPWTFGGGTKLEIK

SEQ ID NO: 8 - protein, 16 aa, synthetic construct
  REGION 1..16: note = 5G6 CDRL1
KSSQSLLDSDGKTYLN

SEQ ID NO: 9 - protein, 7 aa, synthetic construct
  REGION 1..7: note = 5G6 CDRL2
LASKLDS

SEQ ID NO: 10 - protein, 9 aa, synthetic construct
  REGION 1..9: note = 5G6 CDRL3
WQATHFPWT

SEQ ID NO: 11 - DNA, 336 bp, synthetic construct
  misc_feature 1..336: note = 5G6 - Light chain variable region cDNA
  source 1..336: mol_type = other DNA
gatgttgtgatgacccagactccactcactttgtcggttaccattggacaaccagcctccatctcttgcaagtcaagtcagagcctcttagatagtgatggaaagacatatttgaattggttgttacagaggcctggccagtctccaaagcgcctaatctatctggcgtctaaactggactctggagtccctgacaggttcactggcagtggatcagggacagatttcacactgaaaatcagcagagtggaggctgaggatttgggagtttattattgctggcaagctacacattttccgtggacgttcggtggaggcaccaagctggaaatcaaa

SEQ ID NO: 12 - protein, 116 aa, synthetic construct
  REGION 1..116: note = 5G6 heavy chain variable region
EVQLQQSGAELARPGASVKMSCKASGYTFTSYWMHWVKQRPGQGLEWIGAIYPGNSDISYNQKFKGKAKLTAVTSATTAYMELSSLTNEDSAVYYCTLYDYDPDYWGQGTTLTVSS

SEQ ID NO: 13 - protein, 5 aa, synthetic construct
  REGION 1..5: note = 5G6 CDRH1
SYWMH

SEQ ID NO: 14 - protein, 17 aa, synthetic construct
  REGION 1..17: note = 5G6 CDRH2
AIYPGNSDISYNQKFKG

SEQ ID NO: 15 - protein, 7 aa, synthetic construct
  REGION 1..7: note = 5G6 CDRH3
YDYDPDY

SEQ ID NO: 16 - DNA, 348 bp, synthetic construct
  misc_feature 1..348: note = 5G6 - Heavy chain variable region cDNA
  source 1..348: mol_type = other DNA
gaggtccaactgcagcagtctggggctgagctggcaagacctggggcttcagtgaagatgtcctgcaaggcttctggctacacctttaccagctactggatgcactgggtaaaacagaggcctggacagggtctggaatggattggcgctatttatcctggaaatagtgatattagctacaatcagaagttcaagggcaaggccaaactgactgcagtcacatccgccaccactgcctacatggagctcagcagcctaacaaatgaggactctgcggtctattactgtaccctctatgattacgaccctgactactggggccaaggcaccactctcacagtctcctca

SEQ ID NO: 17 - protein, 113 aa, synthetic construct
  REGION 1..113: note = 1A8 light chain variable region
DIVMTQSPSSLAMSVGQKVTMNCKSSQSLLNSSNQKNYLAWFQQKPGQSPKLLVYFASTRESGVPDRFIGSGSGTDFTLTISSVQAEDLADYFCQQHYSTPLTFGAGTKLELK

SEQ ID NO: 18 - protein, 17 aa, synthetic construct
  REGION 1..17: note = 1A8 CDRL1
KSSQSLLNSSNQKNYLA

SEQ ID NO: 19 - protein, 7 aa, synthetic construct
  REGION 1..7: note = 1A8 CDRL2
FASTRES

SEQ ID NO: 20 - protein, 9 aa, synthetic construct
  REGION 1..9: note = 1A8 CDRL3
QQHYSTPLT

SEQ ID NO: 21 - DNA, 339 bp, synthetic construct
  misc_feature 1..339: note = 1A8 light chain variable region cDNA
  source 1..339: mol_type = other DNA
gacattgtgatgacacagtctccatcctccctggctatgtcagtaggacagaaggtcactatgaactgcaagtccagtcagagccttttaaatagtagcaatcaaaagaactatttggcctggttccagcagaaaccaggacagtctcctaaacttctggtatactttgcttccactagggaatctggggtccctgatcgcttcataggcagtggatctgggacagatttcactcttaccatcagcagtgtgcaggctgaagacctggcagattacttctgtcagcaacattatagcactcctctcacgttcggtgctgggaccaagctggagctgaaa

SEQ ID NO: 22 - protein, 114 aa, synthetic construct
  REGION 1..114: note = 1A8 heavy chain variable region
EVQLQQSGAELVRPGALVKLSCKASGFNIKDYYMHWVKQRPEQGLEWIGWIDPENGNTIYDPKFQGKATITADTSSNTAYLQLSSLASEDTAVYYCARGWLLLWGQGTTLTVSS

SEQ ID NO: 23 - protein, 5 aa, synthetic construct
  REGION 1..5: note = 1A8 CDRH1
DYYMH

SEQ ID NO: 24 - protein, 17 aa, synthetic construct
  REGION 1..17: note = 1A8 CDRH2
WIDPENGNTIYDPKFQG

SEQ ID NO: 25 - protein, 5 aa, synthetic construct
  REGION 1..5: note = 1A8 CDRH3
GWLLL

SEQ ID NO: 26 - DNA, 342 bp, synthetic construct
  misc_feature 1..342: note = 1A8 heavy chain variable region cDNA
  source 1..342: mol_type = other DNA
gaggttcagctgcagcagtctggggctgagcttgtgaggccaggggccttagtcaagttgtcctgcaaagcttctggcttcaacattaaagactactatatgcactgggtgaagcagaggcctgaacagggcctggagtggattggatggattgatcctgagaatggtaatactatatatgacccgaagttccagggcaaggccactataacagcagacacatcctccaacacagcctacctgcagctcagcagcctggcatctgaggacactgccgtctattactgtgctagaggatggttactactttggggccaaggcaccactctcacagtctcctca

SEQ ID NO: 27 - protein, 108 aa, synthetic construct
  REGION 1..108: note = 4B3 light chain variable region
EIVLTQSPALMAASPGEKVTITCSVSSSISSSNLHWYQQKSETSPKPWIYGTSNLASGVPVRFSGSGSGTSYSLTISSMEAEDAATYYCQQWSSYPLTFGAGTKLELE

SEQ ID NO: 28 - protein, 12 aa, synthetic construct
  REGION 1..12: note = 4B3 CDRL1
SVSSSISSSNLH

SEQ ID NO: 29 - protein, 7 aa, synthetic construct
  REGION 1..7: note = 4B3 CDRL2
GTSNLAS

SEQ ID NO: 30 - protein, 9 aa, synthetic construct
  REGION 1..9: note = 4B3 CDRL3
QQWSSYPLT

SEQ ID NO: 31 - DNA, 324 bp, synthetic construct
  misc_feature 1..324: note = 4B3 light chain variable region cDNA
  source 1..324: mol_type = other DNA
gaaattgtgctcacccagtctccagcactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccactcacgttcggtgctgggaccaagctggaactggaa

SEQ ID NO: 32 - protein, 119 aa, synthetic construct
  REGION 1..119: note = 4B3 heavy chain variable region
EVQLQQSGPELVKPGSSVKISCKASGYTFTDYNMDWVKQSHGKSLEWIGTINPNNGGTSYNQKFKGKATLTVDKSSNTAYMELRSLTSEDSAVYYCARGYDYDLWFAYWGQGTLVTVSA

SEQ ID NO: 33 - protein, 5 aa, synthetic construct
  REGION 1..5: note = 4B3 CDRH1
DYNMD

SEQ ID NO: 34 - protein, 17 aa, synthetic construct
  REGION 1..17: note = 4B3 CDRH2
TINPNNGGTSYNQKFKG

SEQ ID NO: 35 - protein, 10 aa, synthetic construct
  REGION 1..10: note = 4B3 CDRH3
GYDYDLWFAY

SEQ ID NO: 36 - DNA, 357 bp, synthetic construct
  misc_feature 1..357: note = 4B3 heavy chain variable region cDNA
  source 1..357: mol_type = other DNA
gaggtccagctgcaacagtctggacctgagctggtgaagcctgggtcttcagtgaagatatcctgcaaagcttctggatacacattcactgactacaacatggactgggtgaagcagagccatggaaagagccttgagtggattggtactattaatcctaacaatggtggtactagctacaaccagaagttcaagggcaaggccacattgactgtagacaagtcctccaacacagcctacatggagctccgcagcctgacatctgaggactctgcagtctattactgtgcaagaggctatgattacgacttgtggtttgcttactggggccaagggactctggtcactgtctctgca

SEQ ID NO: 37 - protein, 107 aa, synthetic construct
  REGION 1..107: note = 4E11 light chain variable region
DILMTQSPSSMSVSLGDTVSITCHASQGINSNIGWLLQKPGKSFKGLIYHGTNLEDGVPSRFSGSGSGTDYSLTISSLESEDFADYYCVQYAQFPYTFGGGTKLEIK

SEQ ID NO: 38 - protein, 11 aa, synthetic construct
  REGION 1..11: note = 4E11 CDRL1
HASQGINSNIG

SEQ ID NO: 39 - protein, 7 aa, synthetic construct
  REGION 1..7: note = 4E11 CDRL2
HGTNLED

SEQ ID NO: 40 - protein, 9 aa, synthetic construct
  REGION 1..9: note = 4E11 CDRL3
VQYAQFPYT

SEQ ID NO: 41 - DNA, 321 bp, synthetic construct
  misc_feature 1..321: note = 4E11 light chain variable region cDNA
  source 1..321: mol_type = other DNA
gacatcctgatgacccaatctccatcctccatgtctgtatctctgggagacacagtcagcatcacttgccatgcaagtcagggcattaacagtaatatagggtggttgctgcagaaaccagggaaatcatttaagggcctgatctatcatggaaccaacttggaagatggagttccatcaaggttcagtggcagtggatctggaacagattattctctcaccatcagcagcctggaatctgaggattttgctgactattactgtgtacagtatgctcagtttccgtacacgttcggaggggggaccaaactggaaataaaa

SEQ ID NO: 42 - protein, 116 aa, synthetic construct
  REGION 1..116: note = 4E11 heavy chain variable region
DVQLQESGPGLVKPSQSLSLTCTVTGYSITSDYAWNWIRQFPGNKLEWMGYIGYNGRTSYNPSLKSRISITRDTSKNQFFLQLNYVTTEDTATFYCARLGRGFAYWGQGTLVTVSA

SEQ ID NO: 43 - protein, 6 aa, synthetic construct
  REGION 1..6: note = 4E11 CDRH1
SDYAWN

SEQ ID NO: 44 - protein, 16 aa, synthetic construct
  REGION 1..16: note = 4E11 CDRH2
YIGYNGRTSYNPSLKS

SEQ ID NO: 45 - protein, 7 aa, synthetic construct
  REGION 1..7: note = 4E11 CDRH3
LGRGFAY

SEQ ID NO: 46 - DNA, 348 bp, synthetic construct
  misc_feature 1..348: note = 4E11 heavy chain variable region cDNA
  source 1..348: mol_type = other DNA
gatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataggctacaatggtagaactagttacaacccatctctcaaaagtcgaatctctatcactcgagacacatccaagaaccagttcttcctgcagttgaattatgtgactactgaggacacagccacattttactgtgcaagactgggccgagggtttgcttactggggccaagggactctggtcactgtctctgca

SEQ ID NO: 47 - protein, 108 aa, synthetic construct
  REGION 1..108: note = 5D8 light chain variable region
EIVLTQSPVFMAASPGEKVTITCSVSSSISSSNLHWYQQKSETSPKPWIYGTSNLASGVPVRFSGSGSGTSYSLTISSMEAEDAATYYCQQWSSYPLTFGAGTKLELK

SEQ ID NO: 48 - protein, 12 aa, synthetic construct
  REGION 1..12: note = 5D8 CDRL1
SVSSSISSSNLH

SEQ ID NO: 49 - protein, 7 aa, synthetic construct
  REGION 1..7: note = 5D8 CDRL2
GTSNLAS

SEQ ID NO: 50 - protein, 9 aa, synthetic construct
  REGION 1..9: note = 5D8 CDRL3
QQWSSYPLT

SEQ ID NO: 51 - DNA, 324 bp, synthetic construct
  misc_feature 1..324: note = 5D8 light chain variable region cDNA
  source 1..324: mol_type = other DNA
gaaattgtgctcacccagtctccagtattcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccactcacgttcggtgctgggaccaagctggagctgaaa

SEQ ID NO: 52 - protein, 119 aa, synthetic construct
  REGION 1..119: note = 5D8 heavy chain variable region
EVQLQQSGPDLVKPGSSVKISCKASGYTFTDYNIDWVKQSHGKSLEWIGTINPNYGGTSYNQKFKGKATLTVDKSSSTAYMELRSLTSEDSAVYYCARGYDYDLWFAYWGQGTLVTVSA

SEQ ID NO: 53 - protein, 5 aa, synthetic construct
  REGION 1..5: note = 5D8 CDRH1
DYNID

SEQ ID NO: 54 - protein, 17 aa, synthetic construct
  REGION 1..17: note = 5D8 CDRH2
TINPNYGGTSYNQKFKG

SEQ ID NO: 55 - protein, 10 aa, synthetic construct
  REGION 1..10: note = 5D8 CDRH3
GYDYDLWFAY

SEQ ID NO: 56 - DNA, 357 bp, synthetic construct
  misc_feature 1..357: note = 5D8 heavy chain variable region cDNA
  source 1..357: mol_type = other DNA
gaggtccagctgcaacagtctggacctgacctggtgaagcctgggtcttcagtgaagatttcctgcaaagcttctggatacacattcactgactacaacattgactgggtgaagcagagccatggaaagagccttgagtggattggaactattaatcctaactatggtggtacttcctacaaccagaagttcaagggcaaggccacattgactgtagacaagtcctccagcacagcctacatggagctccgcagcctgacatctgaggactctgcagtctattactgtgcaagaggctatgattacgacttgtggtttgcttactggggccaagggactctggtcactgtctctgca

SEQ ID NO: 57 - protein, 108 aa, synthetic construct
  REGION 1..108: note = 9C9 light chain variable region
EIVLTQSPTLMAASPGEKVTITCSVSSSISSSNLHWYQQKSETSPKPWIYDTSNLASGVPIRFSGSGSGTSYSLTISSVEAEDAATYYCQQWSSYPLTFGSGTKLEIK

SEQ ID NO: 58 - protein, 12 aa, synthetic construct
  REGION 1..12: note = 9C9 CDRL1
SVSSSISSSNLH

SEQ ID NO: 59 - protein, 7 aa, synthetic construct
  REGION 1..7: note = 9C9 CDRL2
DTSNLAS

SEQ ID NO: 60 - protein, 9 aa, synthetic construct
  REGION 1..9: note = 9C9 CDRL3
QQWSSYPLT

SEQ ID NO: 61 - DNA, 324 bp, synthetic construct
  misc_feature 1..324: note = 9C9 light chain variable region cDNA
  source 1..324: mol_type = other DNA
gaaattgtgctcacccagtctccaacactcatggctgcatctccaggggagaaggtcaccatcacctgcagtgtcagctcaagtataagttccagcaacttgcactggtaccagcagaagtcagaaacctcccccaaaccctggatttatgacacatccaacctggcttctggagtccctattcgcttcagtggcagtggatctgggacctcttattctctcacaatcagcagcgtggaggctgaagatgctgccacttattactgtcaacagtggagtagttacccactcacgttcggctcggggacaaagttggaaataaaa

SEQ ID NO: 62 - protein, 114 aa, synthetic construct
  REGION 1..114: note = 9C9 heavy chain variable region
QVQLQQPGAELVKPGASVKLSCKASGYTFTSYWMHWVKQRPGQDLEWIGEIDPSDSYTNYNQKFKGKATLTVDKSSSTAYIQLSSLTSEDSALYYCARFDFAYWGQGTLVTVSA

SEQ ID NO: 63 - protein, 5 aa, synthetic construct
  REGION 1..5: note = 9C9 CDRH1
SYWMH

SEQ ID NO: 64 - protein, 17 aa, synthetic construct
  REGION 1..17: note = 9C9 CDRH2
EIDPSDSYTNYNQKFKG

SEQ ID NO: 65 - protein, 5 aa, synthetic construct
  REGION 1..5: note = 9C9 CDRH3
FDFAY

SEQ ID NO: 66 - DNA, 342 bp, synthetic construct
  misc_feature 1..342: note = 9C9 heavy chain variable region cDNA
  source 1..342: mol_type = other DNA
caggtccaactgcagcagcctggggctgagcttgtgaagcctggggcttcagtgaagctgtcctgcaaggcttctggctacaccttcaccagctactggatgcactgggtgaaacagaggcctggacaagaccttgagtggatcggagagattgatccttctgatagttatactaactacaatcaaaagttcaagggcaaggccacattgactgtagacaaatcctccagcacagcctacattcagctcagcagcctgacatctgaggactctgcgctctattactgtgcaagattcgattttgcttactggggccaagggactctggtcactgtctctgca

SEQ ID NO: 67 - protein, 112 aa, synthetic construct
  REGION 1..112: note = 11B1 light chain 1 (L1) variable region (dominant)
DVVMTQTPLSLPVSLGDQASISCRSSQSLVYSNGNTYLHWYLQKPGQSPKLLIYKVSNRFSGVPDRFSGSGSGTDFTLKISRVEAEDLGVYFCSQSTHVPFTFGSGTKLEIK

SEQ ID NO: 68 - protein, 16 aa, synthetic construct
  REGION 1..16: note = 11B1 L1 CDRL1
RSSQSLVYSNGNTYLH

SEQ ID NO: 69 - protein, 7 aa, synthetic construct
  REGION 1..7: note = 11B1 L1 CDRL2
KVSNRFS

SEQ ID NO: 70 - protein, 9 aa, synthetic construct
  REGION 1..9: note = 11B1 L1 CDRL3
SQSTHVPFT

SEQ ID NO: 71 - DNA, 336 bp, synthetic construct
  misc_feature 1..336: note = 11B1 light chain 1 (L1) variable region cDNA (dominant)
  source 1..336: mol_type = other DNA
gatgttgtgatgacccaaactccactctccctgcctgtcagtcttggagatcaagcctccatctcttgcagatctagtcagagccttgtatatagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccattcacgttcggctcggggacaaagttggaaataaaa

SEQ ID NO: 72 - protein, 108 aa, synthetic construct
  REGION 1..108: note = 11B1 light chain 2 (L2) variable region
ENVLTQSPAIMSASLGEKVTMSCRASSSVNYMYWCQQKSDASPKLWIYYTSNLAPGVPARFSGSGSGNSYSLTISSMEGEDVATYYCQQFTSSPSMHTFGGGTKLEIK

SEQ ID NO: 73 - protein, 10 aa, synthetic construct
  REGION 1..10: note = 11B1 L2 CDRL1
RASSSVNYMY

SEQ ID NO: 74 - protein, 7 aa, synthetic construct
  REGION 1..7: note = 11B1 L2 CDRL2
YTSNLAP

SEQ ID NO: 75 - protein, 11 aa, synthetic construct
  REGION 1..11: note = 11B1 L2 CDRL3
QQFTSSPSMHT

SEQ ID NO: 76 - DNA, 324 bp, synthetic construct
  misc_feature 1..324: note = 11B1 light chain 2 (L2) variable region cDNA
  source 1..324: mol_type = other DNA
gaaaatgtgctcacccagtctccagcaatcatgtctgcatctctaggggagaaggtcaccatgagctgcagggccagctcaagtgtaaattacatgtactggtgccagcagaagtcagatgcctcccccaaactatggatttattacacatccaacctggctcctggagtcccagctcgcttcagtggcagtgggtctgggaactcttattctctcacaatcagcagcatggagggtgaagatgttgccacttattactgccagcagtttactagttccccatccatgcacacgttcggaggggggaccaagctggaaataaaa

SEQ ID NO: 77 - protein, 122 aa, synthetic construct
  REGION 1..122: note = 11B1 heavy chain variable region
QIQLVQSGPELKKPGETVKISCKASGYTFTTAGMQWVKKMPGKGFKWIGWINTHSGDPKYAEDFKGRFAFSLETYASTAYLQISNLKNEDTASYFCARTHIYDGYNYAMDYWGQGTSVTVSS

SEQ ID NO: 78 - protein, 5 aa, synthetic construct
  REGION 1..5: note = 11B1 CDRH1
TAGMQ

SEQ ID NO: 79 - protein, 17 aa, synthetic construct
  REGION 1..17: note = 11B1 CDRH2
WINTHSGDPKYAEDFKG

SEQ ID NO: 80 - protein, 13 aa, synthetic construct
  REGION 1..13: note = 11B1 CDRH3
THIYDGYNYAMDY

SEQ ID NO: 81 - DNA, 366 bp, synthetic construct
  misc_feature 1..366: note = 11B1 heavy chain variable region cDNA
  source 1..366: mol_type = other DNA
cagatccagttggtgcagtctggacctgagctgaagaagcctggagagacagtcaagatctcctgcaaggcttctgggtataccttcacaactgctggaatgcagtgggtaaaaaagatgccaggaaagggttttaagtggattggctggataaacacccactctggagatccaaaatatgcagaagacttcaagggacggtttgccttctctttggaaacctacgccagtactgcatatttgcagataagcaacctcaaaaacgaggacactgcttcgtatttctgtgcgaggacccacatctatgatggttataactatgctatggactactggggtcaagggacctcagtcaccgtctcctca

SEQ ID NO: 82 - protein, 112 aa, synthetic construct; identical to SEQ ID NO: 67
  REGION 1..112: note = 11C8 light chain 1 (L1) variable region (dominant)

SEQ ID NO: 83 - protein, 16 aa, synthetic construct
  REGION 1..16: note = 11C8 L1 CDRL1
RSSQSLVYSNGNTYLH

SEQ ID NO: 84 - protein, 7 aa, synthetic construct
  REGION 1..7: note = 11C8 L1 CDRL2
KVSNRFS

SEQ ID NO: 85 - protein, 9 aa, synthetic construct
  REGION 1..9: note = 11C8 L1 CDRL3
SQSTHVPFT

SEQ ID NO: 86 - DNA, 336 bp, synthetic construct; identical to SEQ ID NO: 71
  misc_feature 1..336: note = 11C8 light chain 1 (L1) variable region cDNA (dominant)
  source 1..336: mol_type = other DNA

SEQ ID NO: 87 - protein, 108 aa, synthetic construct; identical to SEQ ID NO: 72
  REGION 1..108: note = 11C8 light chain 2 (L2) variable region

SEQ ID NO: 88 - protein, 10 aa, synthetic construct
  REGION 1..10: note = 11C8 L2 CDRL1
RASSSVNYMY

SEQ ID NO: 89 - protein, 7 aa, synthetic construct
  REGION 1..7: note = 11C8 L2 CDRL2
YTSNLAP

SEQ ID NO: 90 - protein, 11 aa, synthetic construct
  REGION 1..11: note = 11C8 L2 CDRL3
QQFTSSPSMHT

SEQ ID NO: 91 - DNA, 324 bp, synthetic construct; identical to SEQ ID NO: 76
  misc_feature 1..324: note = 11C8 light chain 2 (L2) variable region cDNA
  source 1..324: mol_type = other DNA

SEQ ID NO: 92 - protein, 122 aa, synthetic construct
  REGION 1..122: note = 11C8 heavy chain variable region
QIQLVQSGPELKKPGETVKISCKASGYTFTTAGMQWVQKMPGKGFKWIGWINTHSGDPKYAEDFKGRFAFSLETYASTAYLQISNLKNEDTASYFCARTHIYDGYNYAMDYWGQGTSVTVSS

SEQ ID NO: 93 - protein, 5 aa, synthetic construct
  REGION 1..5: note = 11C8 CDRH1
TAGMQ

SEQ ID NO: 94 - protein, 17 aa, synthetic construct
  REGION 1..17: note = 11C8 CDRH2
WINTHSGDPKYAEDFKG

SEQ ID NO: 95 - protein, 13 aa, synthetic construct
  REGION 1..13: note = 11C8 CDRH3
THIYDGYNYAMDY

SEQ ID NO: 96 - DNA, 366 bp, synthetic construct
  misc_feature 1..366: note = 11C8 heavy chain variable region cDNA
  source 1..366: mol_type = other DNA
cagatccagttggtgcagtctggacctgagctgaagaagcctggagagacagtcaagatctcctgcaaggcttctgggtataccttcacaactgctggaatgcagtgggtacaaaagatgccaggaaagggttttaagtggattggctggataaacacccactctggagatccaaaatatgcagaagacttcaagggacggtttgccttctctttggaaacctacgccagtactgcatatttgcagataagcaacctcaaaaacgaggacactgcttcgtatttctgtgcgaggacccacatctatgatggttacaactatgctatggactactggggtcaagggacctcagtcaccgtctcctca

SEQ ID NO: 97 - protein, 112 aa, synthetic construct; identical to SEQ ID NO: 67
  REGION 1..112: note = 11H3 light chain variable region

SEQ ID NO: 98 - protein, 16 aa, synthetic construct
  REGION 1..16: note = 11H3 CDRL1
RSSQSLVYSNGNTYLH

SEQ ID NO: 99 - protein, 7 aa, synthetic construct
  REGION 1..7: note = 11H3 CDRL2
KVSNRFS

SEQ ID NO: 100 - protein, 9 aa, synthetic construct
  REGION 1..9: note = 11H3 CDRL3
SQSTHVPFT

SEQ ID NO: 101 - DNA, 336 bp, synthetic construct; identical to SEQ ID NO: 71
  misc_feature 1..336: note = 11H3 light chain variable region cDNA
  source 1..336: mol_type = other DNA

SEQ ID NO: 102 - protein, 122 aa, synthetic construct
  REGION 1..122: note = 11H3 heavy chain variable region
QIQLVQSGPELKKPGETVKISCKASGYTFTTAGMQWVQKMPGKGFKWIGWINTHSGDPKYAEDFKGRFAFSLETYASTAYLQISNLKNEDTATYFCARTHIYDGYNYAMDYWGQGTSVTVSS

SEQ ID NO: 103 - protein, 5 aa, synthetic construct
  REGION 1..5: note = 11H3 CDRH1
TAGMQ

SEQ ID NO: 104 - protein, 17 aa, synthetic construct
  REGION 1..17: note = 11H3 CDRH2
WINTHSGDPKYAEDFKG

SEQ ID NO: 105 - protein, 13 aa, synthetic construct
  REGION 1..13: note = 11H3 CDRH3
THIYDGYNYAMDY

SEQ ID NO: 106 - DNA, 366 bp, synthetic construct
  misc_feature 1..366: note = 11H3 heavy chain variable region cDNA
  source 1..366: mol_type = other DNA
cagatccagttggtgcagtctggacctgagctgaagaagcctggagagacagtcaagatctcctgcaaggcttctgggtataccttcacaactgctggaatgcagtgggtacaaaagatgccaggaaagggttttaagtggattggctggataaacacccactctggagatccaaaatatgcagaagacttcaagggacggtttgccttctctttggaaacctacgccagcactgcatatttgcagataagcaacctcaaaaacgaggacactgctacgtatttctgtgcgaggacccatatctatgatggttataattatgctatggactactggggtcaaggaacctcagtcaccgtctcctca

SEQ ID NO: 107 - protein, 445 aa, synthetic construct
  REGION 1..445: note = 4E11 Heavy chain human IgG1
DVQLQESGPGLVKPSQSLSLTCTVTGYSITSDYAWNWIRQFPGNKLEWMGYIGYNGRTSYNPSLKSRISITRDTSKNQFFLQLNYVTTEDTATFYCARLGRGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 108 - protein, 214 aa, synthetic construct
  REGION 1..214: note = 4E11 Light chain human Kappa
DILMTQSPSSMSVSLGDTVSITCHASQGINSNIGWLLQKPGKSFKGLIYHGTNLEDGVPSRFSGSGSGTDYSLTISSLESEDFADYYCVQYAQFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 445 aa, synthetic construct
  REGION 1..445: note = 5G6 Heavy chain human IgG1
EVQLQQSGAELARPGASVKMSCKASGYTFTSYWMHWVKQRPGQGLEWIGAIYPGNSDISYNQKFKGKAKLTAVTSATTAYMELSSLTNEDSAVYYCTLYDYDPDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 110 - protein, 219 aa, synthetic construct
  REGION 1..219: note = 5G6 Light chain human Kappa
DVVMTQTPLTLSVTIGQPASISCKSSQSLLDSDGKTYLNWLLQRPGQSPKRLIYLASKLDSGVPDRFTGSGSGTDFTLKISRVEAEDLGVYYCWQATHFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 449 aa, synthetic construct
  REGION 1..449: note = 13.1.2 Heavy chain human IgG1
QVQLVESGGGVVQPGRSLRLSCAASGFTFSSYGMHWVRQAPGKGLEWVAVIWYDGSNKYYVDSVKGRFTISRDNSKNTLYLQMNSLRAEDTAVYYCARDGWQQLAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 112 - protein, 219 aa, synthetic construct
  REGION 1..219: note = 13.1.2 Light chain human Kappa
DIVMTQTPLSSPVTLGQPASISCRSSQSLVHSDGNTYLSWLHQRPGQPPRLLIYKISNRFSGVPDRFSGSGAGTAFTLKISRVEAEDVGVYYCMQATQLPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 20 aa, synthetic construct
  REGION 1..20: note = N-terminal light chain signal sequence
MVLQTQVFISLLLWISGAYG

SEQ ID NO: 114 - protein, 19 aa, synthetic construct
  REGION 1..19: note = N-terminal heavy chain signal sequence
MDWTWRILFLVAAATGTHA

SEQ ID NO: 115 - protein, 108 aa, synthetic construct
  REGION 1..108: note = consensus VLs of 4B3 and 5D8
  VARIANT 9: note = Ala, Val, or a conservative amino acid substitution of Ala or Val
  VARIANT 10: note = Leu, Phe, or a conservative amino acid substitution of Leu or Phe
  VARIANT 108: note = Lys, Glu, or a conservative amino acid substitution of Lys or Glu
EIVLTQSPXXMAASPGEKVTITCSVSSSISSSNLHWYQQKSETSPKPWIYGTSNLASGVPVRFSGSGSGTSYSLTISSMEAEDAATYYCQQWSSYPLTFGAGTKLELX

SEQ ID NO: 116 - protein, 119 aa, synthetic construct
  REGION 1..119: note = consensus VHs of 4B3 and 5D8
  VARIANT 10: note = Glu, Asp, or a conservative amino acid substitution of Glu or Asp
  VARIANT 34: note = Met, Ile, or a conservative amino acid substitution of Met or Ile
  VARIANT 55: note = Asn, Tyr, or a conservative amino acid substitution of Asn or Tyr
  VARIANT 77: note = Asn, Ser, or a conservative amino acid substitution of Asn or Ser
EVQLQQSGPXLVKPGSSVKISCKASGYTFTDYNXDWVKQSHGKSLEWIGTINPNXGGTSYNQKFKGKATLTVDKSSXTAYMELRSLTSEDSAVYYCARGYDYDLWFAYWGQGTLVTVSA

SEQ ID NO: 117 - protein, 120 aa, synthetic construct
  REGION 1..120: note = Consensus of VHs of 11B1, 11C8 and 11H3
  VARIANT 38: note = Lys, Gln, or a conservative amino acid substitution thereof
  VARIANT 93: note = Ser, Thr, or a conservative amino acid substitution thereof
QIQLVQSGPELKKPGETVKISCKASGYTFTTAGMQWVXKMPGKGFKWIGWINTHSGDPKYAEDFKGRFAFSLETYASTAYLQISNLKNEDTAXYFCARTHIYDGYNYAMDYWGQGTSVTV

SEQ ID NO: 118 - protein, 108 aa, synthetic construct
  REGION 1..108: note = consensus VLs of 4B3, 5D8 and 9C9
  VARIANT 9: note = Ala, Thr, Val, or a conservative amino acid substitution of Ala,Thr, or Val
  VARIANT 10: note = Leu, Phe, or a conservative amino acid substitution of Leu or Phe
  VARIANT 51: note = Gly, Asp, or a conservative amino acid substitution of Gly or Asp
  VARIANT 61: note = Ile, Val, or a conservative amino acid substitution of Ile or Val
  VARIANT 79: note = Met, Val, or a conservative substitution of Met or Val
  VARIANT 101: note = Ala, Ser, or a conservative amino acid substitution of Ala or Ser
  VARIANT 107: note = Ile, Leu, or a conservative amino acid substitution of Ile or Leu
  VARIANT 108: note = Lys, Glu, or a conservative amino acid substitution of Lys or Glu
EIVLTQSPXXMAASPGEKVTITCSVSSSISSSNLHWYQQKSETSPKPWIYXTSNLASGVPXRFSGSGSGTSYSLTISSXEAEDAATYYCQQWSSYPLTFGXGTKLEXX

SEQ ID NO: 119 - protein, 76 aa, Homo sapiens
  REGION 1..76: note = Amino acid residues 1 to 76 of EGFRvIII
LEEKKGNYVVTDHGSCVRACGADSYEMEEDGVRKCKKCEGPCRKVCNGIGIGEFKDSLSINATNIKHFKNCTSISG

SEQ ID NO: 120 - protein, 62 aa, Homo sapiens
  REGION 1..62: note = Amino acid residues 1 to 62 of EGFRvIII
LEEKKGNYVVTDHGSCVRACGADSYEMEEDGVRKCKKCEGPCRKVCNGIGIGEFKDSLSINA

SEQ ID NO: 121 - protein, 49 aa, Homo sapiens
  REGION 1..49: note = Amino acid residues 1 to 49 of EGFRvIII
LEEKKGNYVVTDHGSCVRACGADSYEMEEDGVRKCKKCEGPCRKVCNGI

SEQ ID NO: 122 - protein, 45 aa, Homo sapiens
  REGION 1..45: note = Amino acid residues 1 to 45 of EGFRvIII
LEEKKGNYVVTDHGSCVRACGADSYEMEEDGVRKCKKCEGPCRKV

SEQ ID NO: 123 - protein, 37 aa, Homo sapiens
  REGION 1..37: note = Amino acid residues 1 to 37 of EGFRvIII
LEEKKGNYVVTDHGSCVRACGADSYEMEEDGVRKCKK

SEQ ID NO: 124 - protein, 33 aa, Homo sapiens
  REGION 1..33: note = Amino acid residues 1 to 33 of EGFRvIII
LEEKKGNYVVTDHGSCVRACGADSYEMEEDGVR

SEQ ID NO: 125 - protein, 18 aa, Homo sapiens
  REGION 1..18: note = Amino acid residues 1 to 18 of EGFRvIII
LEEKKGNYVVTDHGSCVR

SEQ ID NO: 126 - protein, 47 aa, Homo sapiens
  REGION 1..47: note = Amino acid residues 3 to 49 of EGFRvIII
EKKGNYVVTDHGSCVRACGADSYEMEEDGVRKCKKCEGPCRKVCNGI

SEQ ID NO: 127 - protein, 43 aa, Homo sapiens
  REGION 1..43: note = Amino acid residues 3 to 45 of EGFRvIII
EKKGNYVVTDHGSCVRACGADSYEMEEDGVRKCKKCEGPCRKV

SEQ ID NO: 128 - protein, 35 aa, Homo sapiens
  REGION 1..35: note = Amino acid residues 3 to 37 of EGFRvIII
EKKGNYVVTDHGSCVRACGADSYEMEEDGVRKCKK

SEQ ID NO: 129 - protein, 16 aa, Homo sapiens
  REGION 1..16: note = Amino acid residues 3 to 18 of EGFRvIII
EKKGNYVVTDHGSCVR

SEQ ID NO: 130 - protein, 44 aa, Homo sapiens
  REGION 1..44: note = Amino acid residues 6 to 49 of EGFRvIII
GNYVVTDHGSCVRACGADSYEMEEDGVRKCKKCEGPCRKVCNGI

SEQ ID NO: 131 - protein, 40 aa, Homo sapiens
  REGION 1..40: note = Amino acid residues 6 to 45 of EGFRvIII
GNYVVTDHGSCVRACGADSYEMEEDGVRKCKKCEGPCRKV

SEQ ID NO: 132 - protein, 32 aa, Homo sapiens
  REGION 1..32: note = Amino acid residues 6 to 37 of EGFRvIII
GNYVVTDHGSCVRACGADSYEMEEDGVRKCKK

SEQ ID NO: 133 - protein, 40 aa, Homo sapiens
  REGION 1..40: note = Amino acid residues 10 to 49 of EGFRvIII
VTDHGSCVRACGADSYEMEEDGVRKCKKCEGPCRKVCNGI

SEQ ID NO: 134 - protein, 36 aa, Homo sapiens
  REGION 1..36: note = Amino acid residues 10 to 45 of EGFRvIII
VTDHGSCVRACGADSYEMEEDGVRKCKKCEGPCRKV

SEQ ID NO: 135 - protein, 28 aa, Homo sapiens
  REGION 1..28: note = Amino acid residues 10 to 37 of EGFRvIII
VTDHGSCVRACGADSYEMEEDGVRKCKK

SEQ ID NO: 136 - protein, 35 aa, Homo sapiens
  REGION 1..35: note = Amino acid residues 15 to 49 of EGFRvIII
SCVRACGADSYEMEEDGVRKCKKCEGPCRKVCNGI

SEQ ID NO: 137 - protein, 31 aa, Homo sapiens
  REGION 1..31: note = Amino acid residues 15 to 45 of EGFRvIII
SCVRACGADSYEMEEDGVRKCKKCEGPCRKV

SEQ ID NO: 138 - protein, 58 aa, Homo sapiens
  REGION 1..58: note = Amino acid residues 19 to 76 of EGFRvIII
ACGADSYEMEEDGVRKCKKCEGPCRKVCNGIGIGEFKDSLSINATNIKHFKNCTSISG

SEQ ID NO: 139 - protein, 44 aa, Homo sapiens
  REGION 1..44: note = Amino acid residues 19 to 62 of EGFRvIII
ACGADSYEMEEDGVRKCKKCEGPCRKVCNGIGIGEFKDSLSINA

SEQ ID NO: 140 - protein, 31 aa, Homo sapiens
  REGION 1..31: note = Amino acid residues 19 to 49 of EGFRvIII
ACGADSYEMEEDGVRKCKKCEGPCRKVCNGI

SEQ ID NO: 141 - protein, 27 aa, Homo sapiens
  REGION 1..27: note = Amino acid residues 19 to 45 of EGFRvIII
ACGADSYEMEEDGVRKCKKCEGPCRKV

SEQ ID NO: 142 - protein, 19 aa, Homo sapiens
  REGION 1..19: note = Amino acid residues 19 to 37 of EGFRvIII
ACGADSYEMEEDGVRKCKK

SEQ ID NO: 143 - protein, 18 aa, Homo sapiens
  REGION 1..18: note = Amino acid residues 28 to 45 of EGFRvIII
EEDGVRKCKKCEGPCRKV

SEQ ID NO: 144 - protein, 10 aa, Homo sapiens
  REGION 1..10: note = Amino acid residues 28 to 37 of EGFRvIII
EEDGVRKCKK

SEQ ID NO: 145 - protein, 23 aa, synthetic construct
  REGION 1..23: note = Amino acid residues 15 to 37 of EGFRvIII, with Ser15 to Alamutation
ACVRACGADSYEMEEDGVRKCKK

SEQ ID NO: 146 - protein, 23 aa, synthetic construct
  REGION 1..23: note = Amino acid residues 15 to 37 of EGFRvIII, with Cys16 to Alamutation
SAVRACGADSYEMEEDGVRKCKK

SEQ ID NO: 147 - protein, 23 aa, synthetic construct
  REGION 1..23: note = Amino acid residues 15 to 37 of EGFRvIII, with Val17 to Alamutation
SCARACGADSYEMEEDGVRKCKK

SEQ ID NO: 148 - protein, 23 aa, synthetic construct
  REGION 1..23: note = Amino acid residues 15 to 37 of EGFRvIII, with Arg18 to Alamutation
SCVAACGADSYEMEEDGVRKCKK

SEQ ID NO: 149 - protein, 23 aa, synthetic construct
  REGION 1..23: note = Amino acid residues 15 to 37 of EGFRvIII, with Cys20 to Alamutation
SCVRAAGADSYEMEEDGVRKCKK

SEQ ID NO: 150 - protein, 23 aa, synthetic construct
  REGION 1..23: note = Amino acid residues 15 to 37 of EGFRvIII, with Gly21 to Alamutation
SCVRACAADSYEMEEDGVRKCKK

SEQ ID NO: 151 - protein, 23 aa, synthetic construct
  REGION 1..23: note = Amino acid residues 15 to 37 of EGFRvIII, with Asp23 to Alamutation
SCVRACGAASYEMEEDGVRKCKK

SEQ ID NO: 152 - protein, 23 aa, synthetic construct
  REGION 1..23: note = Amino acid residues 15 to 37 of EGFRvIII, with Ser24 to Alamutation
SCVRACGADAYEMEEDGVRKCKK

SEQ ID NO: 153 - protein, 23 aa, synthetic construct
  REGION 1..23: note = Amino acid residues 15 to 37 of EGFRvIII, with Tyr25 to Alamutation
SCVRACGADSAEMEEDGVRKCKK

SEQ ID NO: 154 - protein, 23 aa, synthetic construct
  REGION 1..23: note = Amino acid residues 15 to 37 of EGFRvIII, with Glu26 to Alamutation
SCVRACGADSYAMEEDGVRKCKK

SEQ ID NO: 155 - protein, 23 aa, synthetic construct
  REGION 1..23: note = Amino acid residues 15 to 37 of EGFRvIII, with Met27 to Alamutation
SCVRACGADSYEAEEDGVRKCKK

SEQ ID NO: 156 - protein, 23 aa, synthetic construct
  REGION 1..23: note = Amino acid residues 15 to 37 of EGFRvIII, with Glu28 to Alamutation
SCVRACGADSYEMAEDGVRKCKK

SEQ ID NO: 157 - protein, 23 aa, synthetic construct
  REGION 1..23: note = Amino acid residues 15 to 37 of EGFRvIII, with Glu29 to Alamutation
SCVRACGADSYEMEADGVRKCKK

SEQ ID NO: 158 - protein, 23 aa, synthetic construct
  REGION 1..23: note = Amino acid residues 15 to 37 of EGFRvIII, with Asp30 to Alamutation
SCVRACGADSYEMEEAGVRKCKK

SEQ ID NO: 159 - protein, 23 aa, synthetic construct
  REGION 1..23: note = Amino acid residues 15 to 37 of EGFRvIII, with Gly31 to Alamutation
SCVRACGADSYEMEEDAVRKCKK

SEQ ID NO: 160 - protein, 23 aa, synthetic construct
  REGION 1..23: note = Amino acid residues 15 to 37 of EGFRvIII, with Val32 to Alamutation
SCVRACGADSYEMEEDGARKCKK

SEQ ID NO: 161 - protein, 23 aa, synthetic construct
  REGION 1..23: note = Amino acid residues 15 to 37 of EGFRvIII, with Arg33 to Alamutation
SCVRACGADSYEMEEDGVAKCKK

SEQ ID NO: 162 - protein, 23 aa, synthetic construct
  REGION 1..23: note = Amino acid residues 15 to 37 of EGFRvIII, with Lys34 to Alamutation
SCVRACGADSYEMEEDGVRACKK

SEQ ID NO: 163 - protein, 23 aa, synthetic construct
  REGION 1..23: note = Amino acid residues 15 to 37 of EGFRvIII, with Cys35 to Alamutation
SCVRACGADSYEMEEDGVRKAKK

SEQ ID NO: 164 - protein, 23 aa, synthetic construct
  REGION 1..23: note = Amino acid residues 15 to 37 of EGFRvIII, with Lys36 to Alamutation
SCVRACGADSYEMEEDGVRKCAK

SEQ ID NO: 165 - protein, 23 aa, synthetic construct
  REGION 1..23: note = Amino acid residues 15 to 37 of EGFRvIII, with Lys37 to Alamutation
SCVRACGADSYEMEEDGVRKCKA

SEQ ID NO: 166 - protein, 244 aa, synthetic construct
  REGION 1..244: note = Amino acid sequence for single-chain variable fragment composedof VH, linker, VL sequence derived from 5G6 antibody
  REGION 117..134: note = Example linker sequence including restriction sites; any suitablelinker may be used
EVQLQQSGAELARPGASVKMSCKASGYTFTSYWMHWVKQRPGQGLEWIGAIYPGNSDISYNQKFKGKAKLTAVTSATTAYMELSSLTNEDSAVYYCTLYDYDPDYWGQGTTLTVSSGTGGGSGGGGSGGGGSDVVMTQTPLTLSVTIGQPASISCKSSQSLLDSDGKTYLNWLLQRPGQSPKRLIYLASKLDSGVPDRFTGSGSGTDFTLKISRVEAEDLGVYYCWQATHFPWTFGGGTKLEIK

SEQ ID NO: 167 - protein, 241 aa, synthetic construct
  REGION 1..241: note = Amino acid sequence for single-chain variable fragment composedof VH, linker, VL sequence derived from 4E11 antibody
  REGION 117..134: note = Example linker sequence including restriction sites; any suitablelinker may be used
DVQLQESGPGLVKPSQSLSLTCTVTGYSITSDYAWNWIRQFPGNKLEWMGYIGYNGRTSYNPSLKSRISITRDTSKNQFFLQLNYVTTEDTATFYCARLGRGFAYWGQGTLVTVSAGTGGGSGGGGSGGGGSDVDILMTQSPSSMSVSLGDTVSITCHASQGINSNIGWLLQKPGKSFKGLIYHGTNLEDGVPSRFSGSGSGTDYSLTISSLESEDFADYYCVQYAQFPYTFGGGTKLEIK

SEQ ID NO: 168 - protein, 495 aa, synthetic construct
  REGION 1..495: note = Amino acid exemplary sequence for 5G6-CD28-CD3zeta containingchimeric antigen receptor molecule composed of 5G6 scFV, a CD8hinge, human CD28 transmembrane domain, human CD28-signalingdomain, and human CD3-zeta signaling domain
MLRLLLALNLFPSIQVTGEVQLQQSGAELARPGASVKMSCKASGYTFTSYWMHWVKQRPGQGLEWIGAIYPGNSDISYNQKFKGKAKLTAVTSATTAYMELSSLTNEDSAVYYCTLYDYDPDYWGQGTTLTVSSGTGGGSGGGGSGGGGSDVVMTQTPLTLSVTIGQPASISCKSSQSLLDSDGKTYLNWLLQRPGQSPKRLIYLASKLDSGVPDRFTGSGSGTDFTLKISRVEAEDLGVYYCWQATHFPWTFGGGTKLEIKTTTPAPRPPTPAPTIASQPLSLRPEACRPAAGGAVHTRGLDFACDPSKPFWVLVVVGGVLACYSLLVTVAFIIFWVRSKRSRLLHSDYMNMTPRRPGPTRKHYQPYAPPRDFAAYRSASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 169 - protein, 492 aa, synthetic construct
  REGION 1..492: note = Amino Acid exemplary sequence for 4E11-CD28-CD3zeta containingchimeric antigen receptor molecule composed of 4E11 scFV, a CD8hinge, human CD28 transmembrane domain, human CD28-signalingdomain, and human CD3-zeta signaling domain
MLRLLLALNLFPSIQVTGDVQLQESGPGLVKPSQSLSLTCTVTGYSITSDYAWNWIRQFPGNKLEWMGYIGYNGRTSYNPSLKSRISITRDTSKNQFFLQLNYVTTEDTATFYCARLGRGFAYWGQGTLVTVSAGTGGGSGGGGSGGGGSDVDILMTQSPSSMSVSLGDTVSITCHASQGINSNIGWLLQKPGKSFKGLIYHGTNLEDGVPSRFSGSGSGTDYSLTISSLESEDFADYYCVQYAQFPYTFGGGTKLEIKTTTPAPRPPTPAPTIASQPLSLRPEACRPAAGGAVHTRGLDFACDPSKPFWVLVVVGGVLACYSLLVTVAFIIFWVRSKRSRLLHSDYMNMTPRRPGPTRKHYQPYAPPRDFAAYRSASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 170 - protein, 527 aa, synthetic construct
  REGION 1..527: note = Amino acid sequence for 4E11 bi-specific T cell engager exemplarysequence
  REGION 132..147: note = Example linker sequence; any suitable linker may be used
  REGION 277..284: note = Example linker sequence; any suitable linker may be used
  REGION 285..527: note = CD3-specific scFv engager
  REGION 406..419: note = Example linker sequence; any suitable linker may be used
DVQLQESGPGLVKPSQSLSLTCTVTGYSITSDYAWNWIRQFPGNKLEWMGYIGYNGRTSYNPSLKSRISITRDTSKNQFFLQLNYVTTEDTATFYCARLGRGFAYWGQGTLVTVSAKTTPPSVYPLAPGSLGTGGGSGGGGSGGGGSDVDILMTQSPSSMSVSLGDTVSITCHASQGINSNIGWLLQKPGKSFKGLIYHGTNLEDGVPSRFSGSGSGTDYSLTISSLESEDFADYYCVQYAQFPYTFGGGTKLEIKRADAAPTVSIFPPSSKLGDLGGGGSRDDDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 171 - protein, 19 aa, synthetic construct
  REGION 1..19: note = Exemplary linker
GTGGGGSGGGGSGGGGSDV

SEQ ID NO: 172 - protein, 107 aa, synthetic construct
  REGION 1..107: note = Humanized 4E11 light chain variable region, variant 1 (hVL1)
DIQMTQSPSSLSASVGDRVTITCHASQGINSNIGWYQQKPGKAPKLLIYHGTNLEDGVPSRFSGSGSGTDYTLTISSLQPEDFATYYCVQYAQFPYTFGQGTKLEIK

SEQ ID NO: 173 - protein, 107 aa, synthetic construct
  REGION 1..107: note = Humanized 4E11 light chain variable region, variant 2 (hVL2)
DIQMTQSPSSLSASVGDRVTITCHASQGINSNIGWLQQKPGKAPKGLIYHGTNLEDGVPSRFSGSGSGTDYTLTISSLQPEDFATYYCVQYAQFPYTFGQGTKLEIK

SEQ ID NO: 174 - protein, 107 aa, synthetic construct
  REGION 1..107: note = Humanized 4E11 light chain variable region, variant 3 (hVL3)
DIQMTQSPSSLSASVGDRVTITCHASQGINSNIGWLQQKPGKAFKGLIYHGTNLEDGVPSRFSGSGSGTDYTLTISSLQPEDFATYYCVQYAQFPYTFGQGTKLEIK

SEQ ID NO: 175 - protein, 116 aa, synthetic construct
  REGION 1..116: note = Humanized 4E11 heavy chain variable region, variant 1 (hVH1)
QVQLQESGPGLVKPSQTLSLTCTVSGYSITSDYAWNWIRQPPGKGLEWIGYIGYNGRTSYNPSLKSRVTISVDTSKNQFSLKLSSVTAADTAVYYCARLGRGFAYWGQGTLVTVSS

SEQ ID NO: 176 - protein, 116 aa, synthetic construct
  REGION 1..116: note = Humanized 4E11 heavy chain variable region, variant 2 (hVH2)
QVQLQESGPGLVKPSQTLSLTCTVSGYSITSDYAWNWIRQPPGKGLEWIGYIGYNGRTSYNPSLKSRVTISRDTSKNQFSLKLSSVTAADTAVYYCARLGRGFAYWGQGTLVTVSS

SEQ ID NO: 177 - protein, 116 aa, synthetic construct
  REGION 1..116: note = Humanized 4E11 heavy chain variable region, variant 3 (hVH3)
QVQLQESGPGLVKPSQTLSLTCTVSGYSITSDYAWNWIRQPPGKGLEWMGYIGYNGRTSYNPSLKSRITISRDTSKNQFSLKLSSVTAADTAVYYCARLGRGFAYWGQGTLVTVSS

SEQ ID NO: 178 - protein, 107 aa, synthetic construct [sequence omitted]

SEQ ID NO: 179 - protein, 326 aa, synthetic construct
  REGION 1..326: note = Heavy chain human IgG4 (S228P) constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 180 - protein, 214 aa, synthetic construct
  REGION 1..214: note = Humanized 4E11 light chain, variant 1 (hL1)
  REGION 1..110: note = Variable region
DIQMTQSPSSLSASVGDRVTITCHASQGINSNIGWYQQKPGKAPKLLIYHGTNLEDGVPSRFSGSGSGTDYTLTISSLQPEDFATYYCVQYAQFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - protein, 214 aa, synthetic construct
  REGION 1..214: note = Humanized 4E11 light chain, variant 2 (hL2)
  REGION 1..107: note = Variable region
DIQMTQSPSSLSASVGDRVTITCHASQGINSNIGWLQQKPGKAPKGLIYHGTNLEDGVPSRFSGSGSGTDYTLTISSLQPEDFATYYCVQYAQFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 214 aa, synthetic construct
  REGION 1..214: note = Humanized 4E11 light chain, variant 3 (hL3)
  REGION 1..107: note = Variable region
DIQMTQSPSSLSASVGDRVTITCHASQGINSNIGWLQQKPGKAFKGLIYHGTNLEDGVPSRFSGSGSGTDYTLTISSLQPEDFATYYCVQYAQFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - protein, 442 aa, synthetic construct
  REGION 1..442: note = Humanized 4E11 heavy chain, variant 1 (hH1)
  REGION 1..116: note = Variable region
QVQLQESGPGLVKPSQTLSLTCTVSGYSITSDYAWNWIRQPPGKGLEWIGYIGYNGRTSYNPSLKSRVTISVDTSKNQFSLKLSSVTAADTAVYYCARLGRGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 184 - protein, 442 aa, synthetic construct
  REGION 1..442: note = Humanized 4E11 heavy chain, variant 2 (hH2)
  REGION 1..116: note = Variable region
QVQLQESGPGLVKPSQTLSLTCTVSGYSITSDYAWNWIRQPPGKGLEWIGYIGYNGRTSYNPSLKSRVTISRDTSKNQFSLKLSSVTAADTAVYYCARLGRGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 185 - protein, 442 aa, synthetic construct
  REGION 1..442: note = Humanized 4E11 heavy chain, variant 3 (hH3)
  REGION 1..116: note = Variable region
QVQLQESGPGLVKPSQTLSLTCTVSGYSITSDYAWNWIRQPPGKGLEWMGYIGYNGRTSYNPSLKSRITISRDTSKNQFSLKLSSVTAADTAVYYCARLGRGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2024/0408249 A1
(43) Pub. Date: Dec. 12, 2024

(54) EGFRVIII-TARGETED COMPOUNDS AND USES THEREOF

(71) Applicants: National Research Council of Canada, Ottawa (CA); Fusion Pharmaceuticals Inc., Hamilton (CA)

(72) Inventors: Natalie Grinshtein, Bothell, WA (US); Julie Metcalf, Brantford (CA); Ian R. Duffy, Oakville (CA); William Leslie Turnbull, Burlington (CA); Anne Marcil, Pierrefonds (CA); Maria Jaramillo, Beaconsfield (CA); Traian Sulea, Kirkland (CA); Maria Moreno, Ottawa (CA); Cunle Wu, Montreal (CA)

(21) Appl. No.: 18/695,724

(22) PCT Filed: Sep. 29, 2022
(86) PCT No.: PCT/CA2022/051447

§ 371 Date: Mar. 26, 2024

(30) Foreign Application Priority Data
Sep. 29, 2021 (WO) PCT/CA2021/051360

(51) Int. Cl. A61K 51/10; A61P 35/00; C07D 257/02; C07K 16/28

(52) U.S. Cl. CPC A61K 51/103; A61K 51/1045; A61K 51/1096; A61P 35/00; C07D 257/02; C07K 16/2863; C07K 2317/24; C07K 2317/52; C07K 2317/565; C07K 2317/77

(57) ABSTRACT

Compounds, e.g., radioimmunoconjuguates including a chelating moiety or a metal complex thereof, a linker, and an EGFRVIII targeting moiety. Pharmaceutical compositions of such compounds and methods of treatment for conditions. e.g., cancer, using such compounds or pharmaceutical compositions.

### RELATED APPLICATIONS

The present application claims priority to International Patent Application No. PCT/CA2021/051360, filed Sep. 29, 2021, the entire contents of which are hereby incorporated by reference for all purposes.

### SEQUENCE LISTING

The present specification makes reference to a Sequence Listing (submitted electronically as a .xml file named “FPI_027_Sequence_Listing.xml” on Sep. 29, 2022). The .xml file was generated on Sep. 26, 2022 and is 263 kilobytes in size. The entire contents of the Sequence Listing are herein incorporated by reference.

### BACKGROUND

The epidermal growth factor receptor variant III (EGFRvIII) is amplified, highly expressed and present in 25-64% of glioblastoma multiforme (GBM). EGFRVIII mRNA and protein expression has been detected in a subset of carcinomas of the breast as well as in head and neck squamous cell carcinoma (HNSCC) using multiple complementary techniques. Unlike wild type (wt) epidermal growth factor receptor (EGFR), which is expressed in tissues of epithelial, mesenchymal and neuronal origin and plays a major role in normal cellular processes such as proliferation, differentiation and development, EGFRVIII is not expressed on normal tissues.

The EGFRVIII variant originates from an in-frame deletion of exons 2-7 of the EGFR gene resulting in the removal of a sequence encoding 267 amino acid residues of the extracellular domain. The newly formed splice junction encodes a glycine residue which has no counterpart in wild type human EGFR and therefore forms a neo-epitope. Moreover, numerous studies showed that normal tissues are devoid of EGFRVIII. EGFRVIII thus contains a new tumor specific cell surface epitope that could be exploited for antibody targeted therapies. However, the EGFRvIII neo-epitope is not very immunogenic compared to the remaining of the human sequence, and many of the antibodies generated to date have not been shown to be specifically recognizing EGFRVIII.

Currently known EGFRVIII antibodies include antibody 13.1.2 and ABT-806. Although ABT-806 has been shown to bind preferentially to tumor EGFR in preclinical models, binding of this antibody to wt EGFR present in human skin has been shown to account for the cutaneous toxicity that ABT-806 exhibits in some patients. Antibodies or antigen-binding fragments thereof that specifically target an epitope of EGFRVIII that is absent or not accessible in EGFR-expressing cells would be beneficial for the treatment of cancer patients.

Thus, there remains a need for improved therapeutics (e.g., cancer therapeutics) that can target EGFRvIII.

### SUMMARY

The present disclosure relates to compounds that target epidermal growth factor receptor variant III (EGFRvIII), pharmaceutical compositions thereof, and methods of treating or preventing cancer using such pharmaceutical compositions. In certain embodiments, provided compounds exhibit an increased excretion rate (e.g., after being administered to a mammal) compared to some currently known radiotherapeutics, while still maintaining therapeutic efficacy. In some embodiments, a faster excretion may limit off-target toxicities by limiting the amount of time that the compound stays in a subject. Thus, in some embodiments, provided compounds exhibit reduced off-target toxicities.

In one aspect, provided are compounds comprising the following structure, or pharmaceutically acceptable salts thereof:

A-L¹-(L²)ₙ-B Formula I

wherein
A is a chelating moiety or a metal complex thereof,
B is targeting moiety that is capable of binding to epidermal growth factor receptor variant III (EGFRvIII) or a fragment thereof, wherein the EGFRvIII or fragment thereof comprises a peptide consisting of amino acid residues 1 to 76 of SEQ ID NO: 119;
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
L² each independently has the structure of Formula II:

—X¹-L³-Z¹- Formula II

wherein
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—, or —NR¹—, wherein “*” indicates the attachment point to L³, and R¹ is hydrogen, optionally substituted C₁-C₆ alkyl, optionally substituted C₁-C₆ heteroalkyl, optionally substituted aryl, or optionally substituted heteroaryl;
L³ is optionally substituted C₁-Cso alkyl or optionally substituted C₁-C₅₀ heteroalkyl; and
Z¹ is —CH₂-#, —C(O)-#, —C(S)-#, —OC(O)-#, —C(O)O-#, —NR²C(O)-#, —C(O)NR²-#, or —NR²-#, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl.

In certain embodiments, variable A in Formula I is a chelating moiety selected from the group consisting of DOTA (1,4,7,10-tetraazacyclododecane-1,4,7,10-tetraacetic acid), DOTMA (1R,4R,7R,10R)-α,α′,α″,α′″-tetramethyl-1,4,7,10)-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10)-tetra propionic acid), DO3AM-acetic acid (2-(4,7,10-tris(2-amino-2-oxoethyl)-1,4,7,10-tetraazacyclododecan-1-yl)acetic acid), DOTA-GA anhydride (2,2′,2″-(10)-(2,6-dioxotetrahydro-2H-pyran-3-yl)-1,4,7,10)-tetraazacyclododecane-1,4,7-triyl)triacetic acid, DOTP (1,4,7,10-tetraazacyclododecane-1,4,7,10)-tetra(methylene phosphonic acid)), DOTMP (1,4,6,10)-tetraazacyclodecane-1,4,7,10)-tetramethylene phosphonic acid, DOTA-4AMP (1,4,7,10)-tetraazacyclododecane-1,4,7,10)-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaazacyclohexadecane-1,4,7,10,13,16-hexaacetic acid), PEPA (1,4,7,10,13-pentaazacyclopentadecane-N,N′,N″,N′″N″″-pentaacetic acid), H₄octapa (N,N′-bis(6-carboxy-2-pyridylmethyl)-ethylenediamine-N,N′-diacetic acid), H₂dedpa (1,2-[[6-(carboxy)-pyridin-2-yl]-methylamino]ethane), Hophospa (N,N′-(methylenephosphonate)-N,N′-[6-(methoxycarbonyl)pyridin-2-yl]-methyl-1,2-diaminoethane), TTHA (triethylenetetramine-N,N,N′,N″,N′″N″″-hexaacetic acid), DO2P (tetraazacyclododecane dimethanephosphonic acid), HP-DO3A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), octadentate-HOPO (octadentate hydroxypyridinones), and porphyrin.

In some embodiments, the compound of Formula I is represented by:

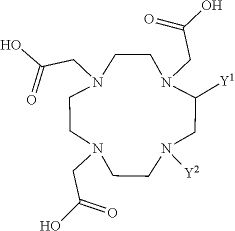

wherein Y¹ is —CH₂OCH₂ (L²)ₙ-B, C═O(L²)ₙ-B, or C═S(L²)ₙ-B and Y² is —CH₂CO₂H; or wherein Y¹ is H and Y² is L¹-(L²)ₙ-B. In certain embodiments, Y¹ is H.

In some embodiments, L¹ is

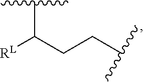

and R^{L} is hydrogen or —CO₂H.

In certain embodiments, X¹ is —C(O)NR¹—* or —NR¹C(O)—*, “*” indicating the attachment point to L³, and R¹ is H.

In certain embodiments, Z¹ is —CH₂—.

In some embodiments, L³ comprises (CH₂CH₂O)₂₋₂₀. In some embodiments, L³ is (CH₂CH₂O)ₘ(CH₂)_{w}, wherein m and w are each independently an integer between 0 and 10 (inclusive), and at least one of m and w is not 0.

In some embodiments, the metal complex comprises a metal selected from the group consisting of Bi, Pb, Y, Mn, Cr, Fe, Co, Zn, Ni, Tc, In, Ga, Cu, Re, a lanthanide, and an actinide. In some embodiments, the metal complex comprises a radionuclide selected from the group consisting of ⁴³Sc, +Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁸⁹Zr, ⁹⁰Y, ⁹⁷Ru, ⁹⁹Tc, ^{99m}Tc, ¹⁰⁵Rh, ¹⁰⁹Pd, ¹¹¹In, ^{117m}Sn, ¹³³La, ¹³⁴Ce, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸ Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ²²⁷Th, and ²²⁹Th.

In some embodiments, variable A is a metal complex of a chelating moiety. In some such embodiments, the metal complex comprises a radionuclide. In some embodiments, the radionuclide is an alpha emitter, e.g., an alpha emitter selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb), or a progeny thereof. In some embodiments, the radionuclide is ⁶⁸Ga, ¹¹¹In, ¹⁷⁷Lu, or ²²⁵Ac. In some embodiments, the radionuclide is 225Ac or a progeny thereof.

In some embodiments, A-L-in Formula I comprises one of the following structures, or a metal complex thereof:

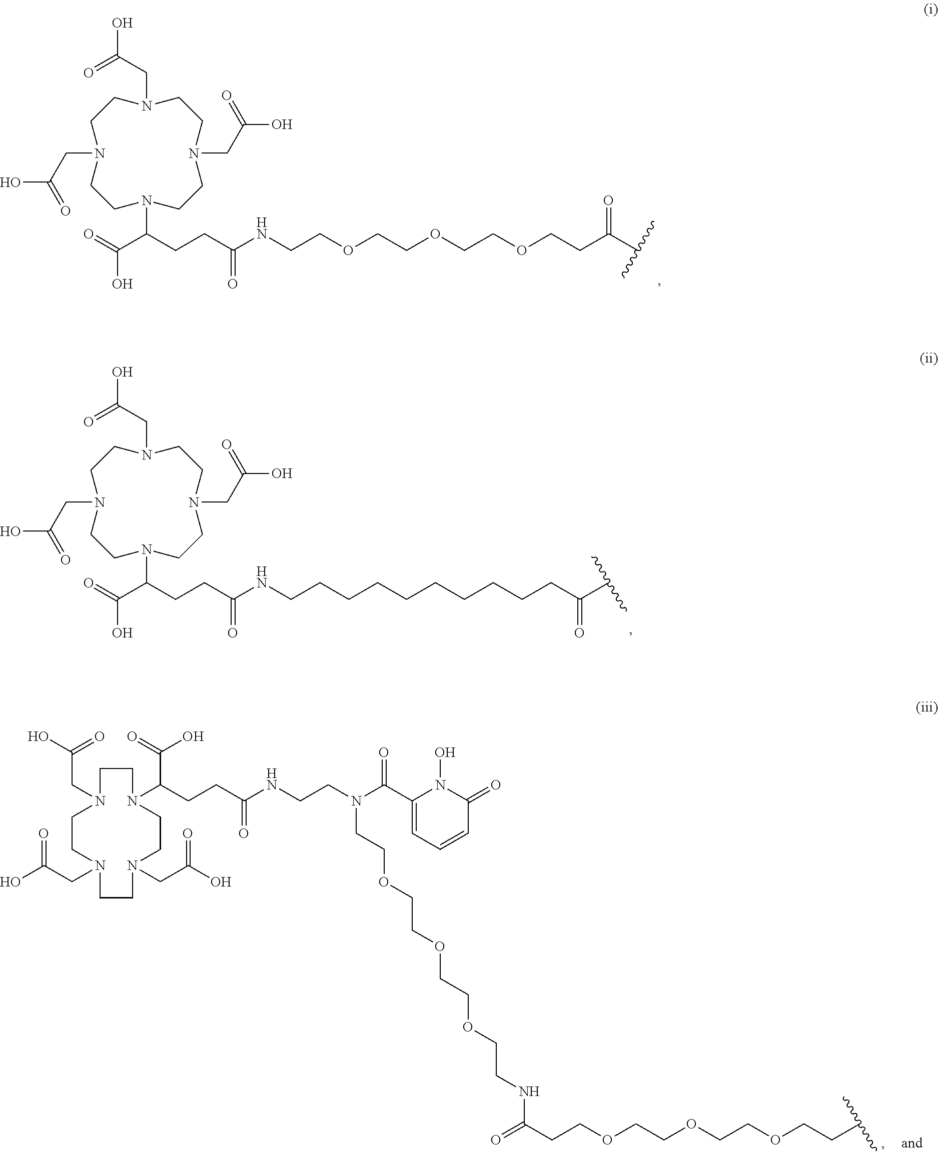 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCNC(=O)CCC(C(=O)O)N1CCN(CC(=O)O)CCN(CC(=O)O)CCN(CC(=O)O)CC1 ; *CCOCCOCCOCCC(=O)NCCOCCOCCOCCN(CCNC(=O)CCC(C(=O)O)N1CCN(CC(=O)O)CCN(CC(=O)O)CCN(CC(=O)O)CC1)C(=O)c1cccc(=O)n1O]

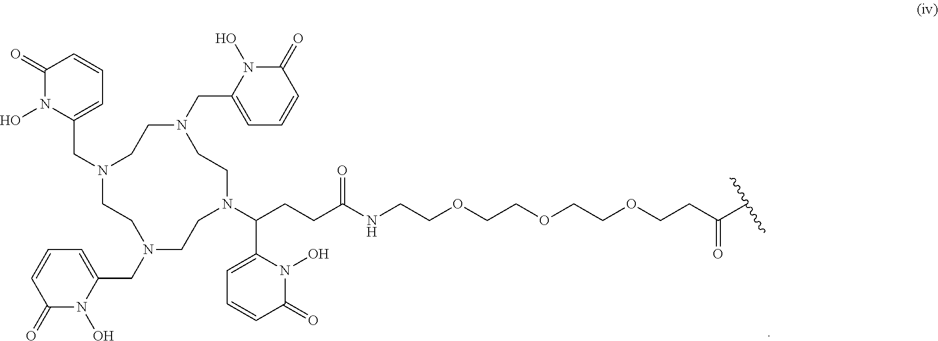 [SMILES: *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

In some embodiments, the compound or a pharmaceutically acceptable salt thereof comprises the following structure, or a metal complex thereof:

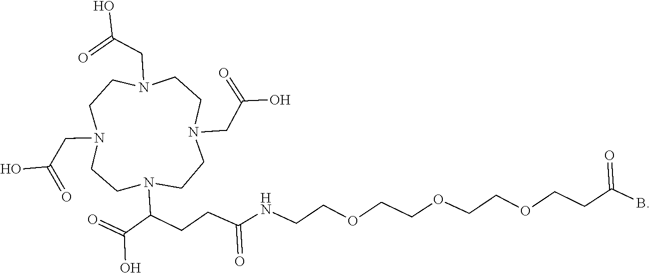

In some embodiments, the targeting moiety comprises an antibody or antigen-binding fragment thereof.

In some embodiments, the antibody or antigen-binding fragment thereof that specifically binds to EGFRvIII or a fragment thereof comprises:
a. a light chain variable region comprising a CDRL1 having the amino acid sequence of SEQ ID NO:8, a CDRL2 sequence of SEQ ID NO:9 and a CDRL3 sequence of SEQ ID NO: 10 and a heavy chain variable region comprising a CDRH1 sequence of SEQ ID NO: 13, a CDRH2 sequence of SEQ ID NO:14 and a CDRH3 sequence of SEQ ID NO: 15;
b. a light chain variable region comprising a CDRL1 having the amino acid sequence set forth in SEQ ID NO:18, a CDRL2 having the amino acid sequence set forth in SEQ ID NO: 19 and a CDRL3 having the amino acid sequence set forth in SEQ ID NO:20 and a heavy chain variable region comprising a CDRH1 having the amino acid sequence set forth in SEQ ID NO:23, a CDRH2 having the amino acid sequence set forth in SEQ ID NO: 24 and a CDRH3 having the amino acid sequence set forth in SEQ ID NO:25;
c. a light chain variable region comprising a CDRL1 having the amino acid sequence set forth in SEQ ID NO:28, a CDRL2 having the amino acid sequence set forth in SEQ ID NO: 29 and a CDRL3 having the amino acid sequence set forth in SEQ ID NO:30 and a heavy chain variable region comprising a CDRH1 having the amino acid sequence set forth in SEQ ID NO:33, a CDRH2 having the amino acid sequence set forth in SEQ ID NO: 34 and a CDRH3 having the amino acid sequence set forth in SEQ ID NO:35;
d. a light chain variable region comprising a CDRL1 having the amino acid sequence set forth in SEQ ID NO:38, a CDRL2 having the amino acid sequence set forth in SEQ ID NO: 39 and a CDRL3 having the amino acid sequence set forth in SEQ ID NO:40 and a heavy chain variable region comprising a CDRH1 having the amino acid sequence set forth in SEQ ID NO:43, a CDRH2 having the amino acid sequence set forth in SEQ ID NO: 44 and a CDRH3 having the amino acid sequence set forth in SEQ ID NO:45;
e. a light chain variable region comprising a CDRL1 having the amino acid sequence set forth in SEQ ID NO:48, a CDRL2 having the amino acid sequence set forth in SEQ ID NO: 49 and a CDRL3 having the amino acid sequence set forth in SEQ ID NO:50 and a heavy chain variable region comprising a CDRH1 having the amino acid sequence set forth in SEQ ID NO:53, a CDRH2 having the amino acid sequence set forth in SEQ ID NO: 54 and a CDRH3 having the amino acid sequence set forth in SEQ ID NO:55;
f. a light chain variable region comprising a CDRL1 having the amino acid sequence set forth in SEQ ID NO:58, a CDRL2 having the amino acid sequence set forth in SEQ ID NO: 59 and a CDRL3 having the amino acid sequence set forth in SEQ ID NO:60 and a heavy chain variable region comprising a CDRH1 having the amino acid sequence set forth in SEQ ID NO:63, a CDRH2 having the amino acid sequence set forth in SEQ ID NO: 64 and a CDRH3 having the amino acid sequence set forth in SEQ ID NO:65;
g. a light chain variable region comprising a CDRL1 having the amino acid sequence set forth in SEQ ID NO:68, a CDRL2 having the amino acid sequence set forth in SEQ ID NO: 69 and a CDRL3 having the amino acid sequence set forth in SEQ ID NO: 70 and a heavy chain variable region comprising a CDRH1 having the amino acid sequence set forth in SEQ ID NO:78, a CDRH2 having the amino acid sequence set forth in SEQ ID NO: 79 and a CDRH3 having the amino acid sequence set forth in SEQ ID NO:80; or
h. a light chain variable region comprising a CDRL1 having the amino acid sequence set forth in SEQ ID NO:73, a CDRL2 having the amino acid sequence set forth in SEQ ID NO: 74 and a CDRL3 having the amino acid sequence set forth in SEQ ID NO: 75 and a heavy chain variable region comprising a CDRH1 having the amino acid sequence set forth in SEQ ID NO:78, a CDRH2 having the amino acid sequence set forth in SEQ ID NO: 79 and a CDRH3 having the amino acid sequence set forth in SEQ ID NO:80.

In some embodiments, the antibody or antigen-binding fragment thereof that specifically binds to EGFRVIII or a fragment thereof comprises:
a. a light chain variable region comprising an amino acid sequence at least 80% identical to the amino acid sequence set forth in SEQ ID NO: 118 or substantially identical to SEQ ID NO: 118 and a heavy chain variable region comprising an amino acid sequence at least 80% identical to the amino acid sequence set forth in SEQ ID NO: 116 or substantially identical to SEQ ID NO: 116;
b. a light chain variable region comprising an amino acid sequence at least 80% identical to the amino acid sequence set forth in SEQ ID NO: 115 or substantially identical to SEQ ID NO: 115 and a heavy chain variable region comprising an amino acid sequence at least 80% identical to the amino acid sequence set forth in SEQ ID NO:116 or substantially identical to SEQ ID NO: 116; or
c. a light chain variable region comprising an amino acid sequence at least 80% identical to the amino acid sequence set forth in SEQ ID NO: 118 or substantially identical to SEQ ID NO: 118 and a heavy chain variable region comprising an amino acid sequence at least 80% identical to the amino acid sequence set forth in SEQ ID NO:62 or substantially identical to SEQ ID NO:62.

In some embodiments, the antibody or antigen-binding fragment thereof comprises:
a. a light chain variable region comprising a sequence at least 80% identical to the amino acid sequence set forth in SEQ ID NO:7 or substantially identical to SEQ ID NO:7 and a heavy chain variable region comprising a sequence at least 80% identical to the amino acid sequence set forth in SEQ ID NO: 12 or substantially identical to SEQ ID NO: 12;
b. a light chain variable region comprising an amino acid sequence at least 80% identical to the amino acid sequence set forth in SEQ ID NO: 17 or substantially identical to SEQ ID NO: 17 and a heavy chain variable region comprising an amino acid sequence at least 80% identical to the amino acid sequence set forth in SEQ ID NO:22 or substantially identical to SEQ ID NO:22;
c. a light chain variable region comprising an amino acid sequence at least 80% identical to the amino acid sequence set forth in SEQ ID NO:27 or substantially identical to SEQ ID NO: 27 and a heavy chain variable region comprising an amino acid sequence at least 80% identical to the amino acid sequence set forth in SEQ ID NO:32 or substantially identical to SEQ ID NO:32;
d. a light chain variable region comprising an amino acid sequence at least 80% identical to the amino acid sequence set forth in SEQ ID NO: 37 or substantially identical to SEQ ID NO: 37 and a heavy chain variable region comprising an amino acid sequence at least 80% identical to the amino acid sequence set forth in SEQ ID NO:42 or substantially identical to SEQ ID NO:42;
e. a light chain variable region comprising an amino acid sequence at least 80% identical to the amino acid sequence set forth in SEQ ID NO:47 or substantially identical to SEQ ID NO: 47 and a heavy chain variable region comprising an amino acid sequence at least 80% identical to the amino acid sequence set forth in SEQ ID NO:52 or substantially identical to SEQ ID NO:52;
f. a light chain variable region comprising an amino acid sequence at least 80% identical to the amino acid sequence set forth in SEQ ID NO:57 or substantially identical to SEQ ID NO: 57 and a heavy chain variable region comprising an amino acid sequence at least 80% identical to the amino acid sequence set forth in SEQ ID NO:62 or substantially identical to SEQ ID NO:62;
g. a light chain variable region comprising an amino acid sequence at least 80% identical to the amino acid sequence set forth in SEQ ID NO:67 or substantially identical to SEQ ID NO: 67 and a heavy chain variable region comprising an amino acid sequence at least 80% identical to the amino acid sequence set forth in SEQ ID NO:77 or substantially identical to SEQ ID NO:77, the amino acid set forth in SEQ ID NO:92 or substantially identical to SEQ ID NO:92 or the amino acid sequence set forth in SEQ ID NO:102 or substantially identical to SEQ ID NO: 102; or
h. a light chain variable region comprising an amino acid sequence at least 80% identical to the amino acid sequence set forth in SEQ ID NO:72 or substantially identical to SEQ ID NO: 72 and a heavy chain variable region comprising an amino acid sequence at least 80% identical to the amino acid sequence set forth in SEQ ID NO:77 or substantially identical to SEQ ID NO: 77 or the amino acid set forth in SEQ ID NO:92 or substantially identical to SEQ ID NO:92.

In some embodiments, the antibody or antigen-binding fragment thereof comprises:
a. a light chain variable region having the amino acid sequence set forth in SEQ ID NO: 172 and a heavy chain variable region having the amino acid sequence set forth in SEQ ID NO: 175;
b. a light chain variable region having the amino acid sequence set forth in SEQ ID NO: 173 and a heavy chain variable region having the amino acid sequence set forth in SEQ ID NO: 175;
c. a light chain variable region having the amino acid sequence set forth in SEQ ID NO: 174 and a heavy chain variable region having the amino acid sequence set forth in SEQ ID NO: 175;
d. a light chain variable region having the amino acid sequence set forth in SEQ ID NO: 172 and a heavy chain variable region having the amino acid sequence set forth in SEQ ID NO: 176;
e. a light chain variable region having the amino acid sequence set forth in SEQ ID NO: 173 and a heavy chain variable region having the amino acid sequence set forth in SEQ ID NO: 176;
f. a light chain variable region having the amino acid sequence set forth in SEQ ID NO: 174 and a heavy chain variable region having the amino acid sequence set forth in SEQ ID NO: 176;
g. a light chain variable region having the amino acid sequence set forth in SEQ ID NO: 172 and a heavy chain variable region having the amino acid sequence set forth in SEQ ID NO: 177;
h. a light chain variable region having the amino acid sequence set forth in SEQ ID NO: 173 and a heavy chain variable region having the amino acid sequence set forth in SEQ ID NO: 177; or
i. a light chain variable region having the amino acid sequence set forth in SEQ ID NO: 174 and a heavy chain variable region having the amino acid sequence set forth in SEQ ID NO: 177.

In certain embodiments, the antibody or antigen-binding fragment thereof comprises:
a. a light chain variable region having the amino acid sequence set forth in SEQ ID NO: 174 and a heavy chain variable region having the amino acid sequence set forth in SEQ ID NO: 176; or
b. a light chain variable region having the amino acid sequence set forth in SEQ ID NO: 172 and a heavy chain variable region having the amino acid sequence set forth in SEQ ID NO: 177:

In some embodiments, the antibody or antigen-binding fragment thereof comprises:
a. a light chain region having the amino acid sequence set forth in SEQ ID NO: 180 and a heavy chain region having the amino acid sequence set forth in SEQ ID NO: 183;
b. a light chain region having the amino acid sequence set forth in SEQ ID NO: 181 and a heavy chain region having the amino acid sequence set forth in SEQ ID NO: 183;
c. a light chain region having the amino acid sequence set forth in SEQ ID NO: 182 and a heavy chain region having the amino acid sequence set forth in SEQ ID NO: 183;
d. a light chain region having the amino acid sequence set forth in SEQ ID NO: 180 and a heavy chain region having the amino acid sequence set forth in SEQ ID NO: 184;
e. a light chain region having the amino acid sequence set forth in SEQ ID NO: 181 and a heavy chain region having the amino acid sequence set forth in SEQ ID NO: 184;
f. a light chain region having the amino acid sequence set forth in SEQ ID NO: 182 and a heavy chain region having the amino acid sequence set forth in SEQ ID NO: 184;
g. a light chain region having the amino acid sequence set forth in SEQ ID NO: 180 and a heavy chain region having the amino acid sequence set forth in SEQ ID NO: 185;
h. a light chain region having the amino acid sequence set forth in SEQ ID NO: 181 and a heavy chain region having the amino acid sequence set forth in SEQ ID NO: 185; or
i. a light chain region having the amino acid sequence set forth in SEQ ID NO: 182 and a heavy chain region having the amino acid sequence set forth in SEQ ID NO: 185.

In some embodiments, the antibody is a monoclonal antibody, a polyclonal antibody, a humanized antibody, a chimeric antibody, a human antibody, a single chain antibody, or a multispecific antibody. In some embodiments, the antibody is a humanized monoclonal antibody.

In some embodiments, the antibody or antigen-binding fragment thereof comprises a human IgG1 constant region.

In some embodiments, the antibody or antigen-binding fragment thereof comprises a human IgG2 constant region.

In some embodiments, the antibody or antigen-binding fragment thereof comprises a human IgG4 constant region. In certain embodiments, the antibody or antigen-binding fragment thereof comprises a human IgG4 (S228P) constant region comprising a human kappa light chain constant region having the amino acid sequence set forth in SEQ ID NO: 178 and a human IgG4 (S228P) heavy chain constant region having the amino acid sequence set forth in SEQ ID NO: 179.

In some embodiments, the antibody or antigen-binding fragment thereof comprises:
a. a light chain comprising an amino acid sequence at least 80% identical to the amino acid sequence set forth in SEQ ID NO: 108 or substantially identical to SEQ ID NO:108 and a heavy chain comprising an amino acid sequence at least 80% identical to the amino acid sequence set forth in SEQ ID NO: 107 or substantially identical to SEQ ID NO:107; or
b. a light chain comprising an amino acid sequence at least 80% identical to the amino acid sequence set forth in SEQ ID NO:110 or substantially identical to SEQ ID NO:110 and a heavy chain comprising an amino acid sequence at least 80% identical to the amino acid sequence set forth in SEQ ID NO:109 or substantially identical to SEQ ID NO:109.

In some embodiments, the antigen-binding fragment comprises a scFv, a Fab, a Fab′ or a (Fab′)₂.

In some embodiments, the antibody or antigen-binding fragment thereof comprises a light chain variable region comprising a CDRL1 having the amino acid sequence set forth in SEQ ID NO:38, a CDRL2 having the amino acid sequence set forth in SEQ ID NO: 39 and a CDRL3 having the amino acid sequence set forth in SEQ ID NO:40 and a heavy chain variable region comprising a CDRH1 having the amino acid sequence set forth in SEQ ID NO: 43, a CDRH2 having the amino acid sequence set forth in SEQ ID NO:44 and a CDRH3 having the amino acid sequence set forth in SEQ ID NO:45.

In some embodiments, the compound comprises the following structure:

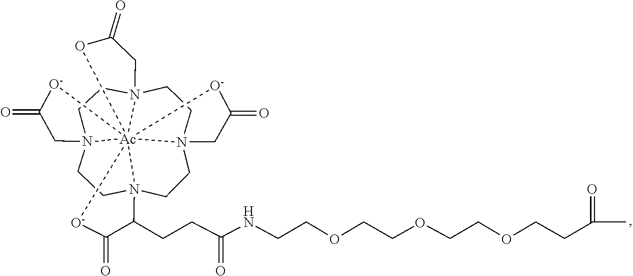

wherein · is an antibody or an antigen-binding fragment thereof that specifically binds to EGFRvIII or a fragment thereof. In some embodiments, the antibody or an antigen-binding fragment thereof is linked to A-L-via the side-chain amino group of a lysine residue

In another aspect, the present invention also relates to a pharmaceutical composition comprising one of the compounds described above and a pharmaceutically acceptable carrier, diluent, or excipient.

Still within the scope of this invention is a method of radiation treatment planning and/or radiation treatment of cancer and the method comprising administering to a subject in need thereof one of the compounds set forth above or a pharmaceutical composition comprising the same.

Also within the scope of this invention is a method of treating cancer that comprises expressing EGFRvIII, the method comprising administering to a subject (e.g., a human) in need thereof a compound or pharmaceutical composition provided herein in a therapeutically effective amount.

Further included in this invention is a method of treating or preventing cancer that comprises cells expressing EGFRVIII, the method comprising administering to a subject (e.g., a human) in need thereof a first dose of a compound or pharmaceutical composition provided above in an amount effective for radiation treatment planning, followed by administering subsequent doses of a compound or pharmaceutical composition provided above in a therapeutically effective amount.

In some embodiments, the compound or composition administered in the first dose and the compound or composition administered a subsequent dose are the same.

In some embodiments, the compound or composition administered in the first dose and the compound or composition administered a subsequent are different.

In some embodiments, the cancer that comprises cells expressing EGFRVIII is glioblastoma multiforme or carcinoma.

In some embodiments, the method of treatment further comprises administering to a subject (e.g., a human) in need thereof an antiproliferative agent, radiation sensitizer, an immunoregulatory or immunomodulatory agent.

In some embodiments, the method of treatment comprises administering the compound or pharmaceutical composition described herein in combination with an antiproliferative agent, in the absence or presence of external beam radiation. In certain embodiments, the antiproliferative agent is temozolomide (TMZ).

In some embodiments, the method of treatment comprises administering a therapeutically effective amount of a compound or pharmaceutical composition described herein in multiple doses (e.g., dosing once weekly for 4 cycles or dosing once every two weeks for 2 cycles).

In some embodiments, the method of treatment comprises administering a therapeutically effective amount of a compound or pharmaceutical composition described herein in a single dose.

### BRIEF DESCRIPTION OF THE DRAWINGS

FIG. 1A is a schematic depicting the general structure of bifunctional chelate comprising a chelate, a linker, and a cross-linking group.

FIG. 1B is a schematic depicting the general structure of a bifunctional conjugate comprising a chelate, a linker, and a targeting moiety.

FIG. 1C and FIG. 1D are schematics depicting the structures of [¹⁷⁷Lu]-DOTA-anti-EGFRvIII and [²²⁵Ac]-DOTA-anti-EGFRvIII, two exemplary EGFRvIII radioimmunoconjugates disclosed herein.

FIG. 2 is a schematic depicting the synthesis of the bifunctional chelate, 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B). Synthesis of Compound B is described in Example 2.

FIG. 3 is a schematic depicting the synthesis of the bifunctional chelate, 4-{[2-(2-{2-[3-oxo-3-(2,3,5,6-tetrafluorophenoxy) propoxy]ethoxy}ethoxy)ethyl]carbamoyl}-2-[4,7,10-tris(carboxymethyl)-1,4,7,10-tetraazacyclododecan-1-yl]butanoic acid (Compound C). Synthesis of Compound C is described in Example 3.

FIG. 4 is a schematic depicting the conjugation and radiolabeling for synthesis of [¹⁷⁷Lu]-Compound C-anti-EGFRvIII conjugate. See Example 4.

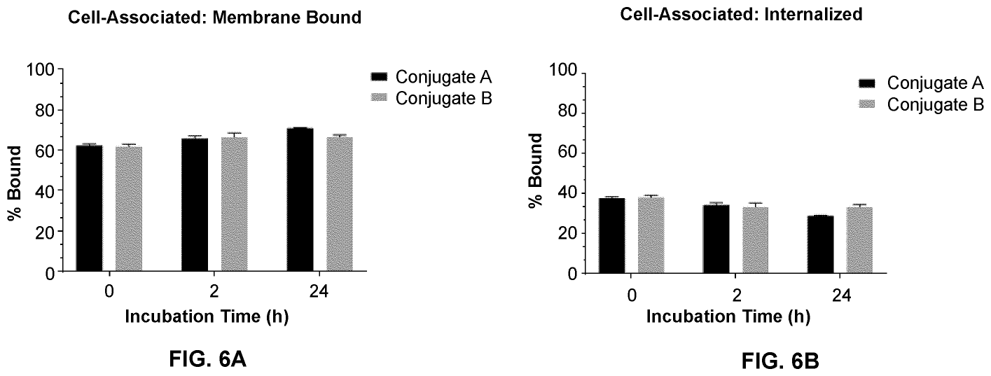

FIGS. 5A and 5B are binding curves for [¹⁷⁷Lu]-Compound C-anti-EGFRvIII conjugates, i.e., Conjugates A and B, respectively, binding to U87-EGFRvIII cells. TB represents total binding, SB represents specific binding, and NSB represents non-specific binding. See Example 6.

FIGS. 6A and 6B shows the results of internalization of Conjugates A and B in U87-EGFRvIII cells. See Example 7.

FIGS. 7A-7C shows the results of residualization of Conjugates A and B in U87-EGFRvIII cells. See Example 7.

FIGS. 8A and 8B shows a plot representing the results of biodistribution studies in a subcutaneous U87-EGFRVIII model and injected with Conjugates A and B, respectively. Percentage injected dose per gram of tissue (% ID/g) is plotted on the x-axis and is shown for blood, heart, intestines, kidneys, liver, lungs, spleen, tumor, urine, and tail at 4, 24, 96, and 168 hours. See Example 8.

FIG. 9 shows a plot representing the results of biodistribution studies in a U87-EGFRvIII-GFP-Luc orthotopic model and injected with Conjugates A and B. Percentage injected dose per gram of tissue (% ID/g) is plotted on the x-axis and is shown for blood, tumor, normal brain, spleen, liver, kidneys, and tail at 96 hours. See Example 9.

FIGS. 10A-10D show single photon emission computed tomography/computer tomography (SPECT/CT) images (FIGS. 10A and 10C) and plots (FIGS. 10B and 10D) representing the results of biodistribution studies in an Orthotopic G39-GFP-Luc model, injected with [¹¹¹In]-Compound C-anti-EGFRvIII (FIGS. 10A and 10B) or [¹¹¹In]-Compound C-IgG4 conjugates (FIGS. 10C and 10D). In the plots shown in FIGS. 10B and 10D, percentage injected dose per cubic centimeter of tissue (% ID/cc) is plotted on the y-axis and is shown for tumor, normal brain, bladder, kidneys, liver, spleen, heart, lung, GI, and bone at 96 hours. See Example 10.

FIGS. 11A-11D show single photon emission computed tomography/computer tomography (SPECT/CT) images (FIGS. 11A and 11C) and plots (FIGS. 11B and 11D) representing the results of biodistribution studies in an Orthotopic G06-GFP-Luc model, injected with [¹¹¹In]-Compound C-anti-EGFRvIII (FIGS. 11A and 11B) or [¹¹¹In]-Compound C-IgG4 conjugates (FIGS. 11C and 11D). In the plots shown in FIGS. 11B and 11D, percentage injected dose per cubic centimeter of tissue (% ID/cc) is plotted on the y-axis and is shown for tumor, normal brain, bladder, kidneys, liver, spleen, heart, lung, GI, and bone at 96 hours. See Example 10.

FIGS. 12A-12C show results of in vivo efficacy studies in a U87-EGFRvIII-GFP-Luc orthotopic model and injected with [²²⁵Ac]-Compound C-anti-EGFRvIII at single doses of 100 nCi and 200 nCi. FIG. 12A is a plot showing the bioluminescence (indicative of presence of tumors measured at certain timepoints after treatment; FIG. 12B shows a survival curve; and FIG. 12C is a table depicting the median survival and survival benefit in each treatment group. See Example 11.

FIGS. 13A and 13B shows results of in vivo efficacy studies in an Orthotopic G06-GFP-Luc model and injected with [²²⁵Ac]-Compound C-anti-EGFRvIII at single doses of 100 nCi and 200 nCi. FIG. 13A shows a survival curve, and FIG. 13B is a table depicting the median survival and survival benefit in each treatment group. See Example 11.

FIGS. 14A and 14B shows results of in vivo efficacy studies in an Orthotopic G39-GFP-Luc model and injected with [²²⁵Ac]-Compound C-anti-EGFRvIII at a single dose of 100, 200, or 400 nCi or a total cumulative radiochemical dose of 400 nCi fractionated as follows: 100 nCi dosed once weekly for 4 weeks (100 nCi×4) or 200 nCi dosed every 2 weeks for 2 doses (200 nCi×2). FIG. 14A shows a survival curve, and FIG. 14B is a table depicting the median survival and survival benefit in each treatment group. See Example 12.

FIGS. 15A and 15B shows a results of in vivo efficacy studies in an Orthotopic G06-GFP-Luc model and injected with [²²⁵Ac]-Compound C-anti-EGFRvIII at 200 nCi in combination with standard of care (external beam radiation combined with temozolomide). FIG. 15A shows a survival curve, and FIG. 15B is a table depicting the median survival and survival benefit in each treatment group. See Example 13.

### DETAILED DESCRIPTION

Radioimmunoconjugates are designed to target a protein or receptor that is upregulated in a disease state to deliver a radioactive payload to damage and kill cells of interest (radioimmunotherapy). The process of delivering such a payload, via radioactive decay, produces an alpha, beta, or gamma particle or Auger electron that can cause direct effects to DNA (such as single or double stranded DNA breaks) or indirect effects such as by-stander or crossfire effects.

Radioimmunoconjugates typically contain a targeting moiety (e.g., a biological targeting moiety (e.g., an antibody or antigen binding fragment thereof)) that is capable of specifically binding to human EGFRVIII, a radioisotope, and a molecule that links the two. Conjugates are formed when a bifunctional chelate is appended to the targeting molecule (e.g., biological targeting molecule) so that structural alterations are minimal while maintaining target affinity. Once radiolabeled, the final radioimmunoconjugate is formed.

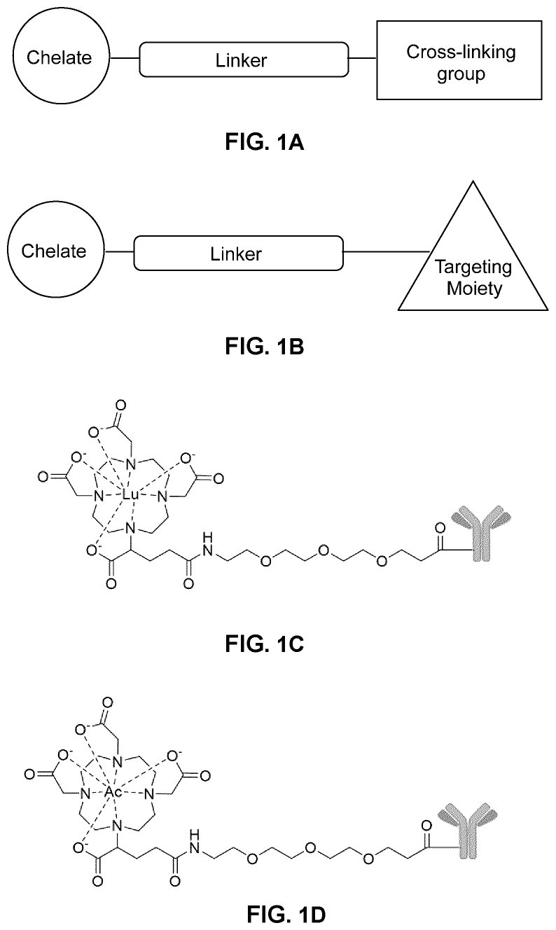

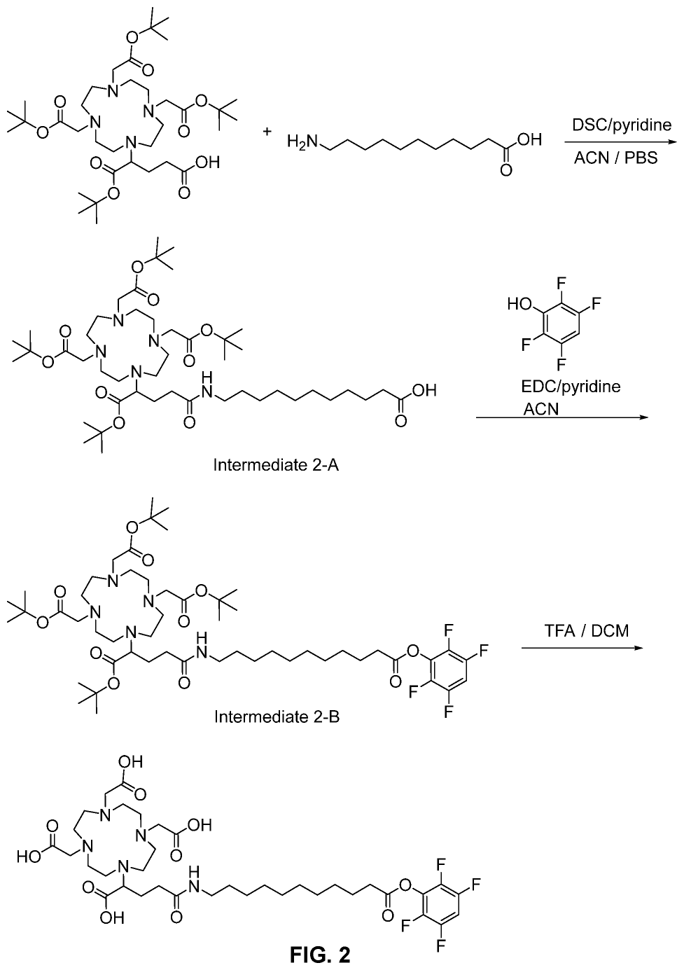

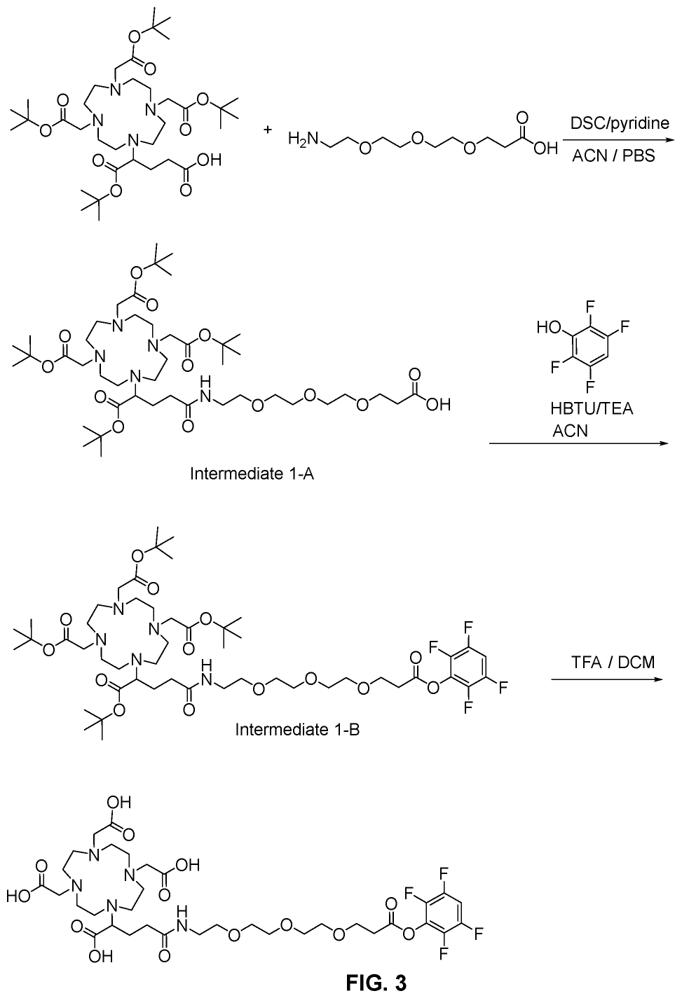

Bifunctional chelates structurally contain a chelate, a linker, and a cross-linking group (FIG. 1A). When developing new bifunctional chelates, most efforts focus on the chelating portion of the molecule. Several examples of bifunctional chelates have been described with various cyclic and acyclic structures conjugated to a targeted moiety. [Bioconjugate Chem. 2000, 11, 510-519; Bioconjugate Chem. 2012, 23, 1029-1039; Mol Imaging Biol. 2011, 13, 215-221, Bioconjugate Chem. 2002, 13, 110-115.]

One of the key factors of developing safe and effective radioimmunoconjugates is maximizing efficacy while minimizing off-target toxicity in normal tissue. While this statement is one of the core tenets of developing new drugs, the application to radioimmunotherapeutics presents new challenges. Radioimmunoconjugates do not need to block a receptor, as needed with a therapeutic antibody, or release the cytotoxic payload intracellularly, as required by an antibody drug conjugate (“ADC”), in order to have therapeutic efficacy. However, the emission of the toxic particle is an event that occurs as a result of first-order (radioactive) decay and can occur at random anywhere inside the body after administration. Once the emission occurs, damage could occur to surrounding cells within the range of the emission leading to the potential of off-target toxicity. Therefore, limiting exposure of these emissions to normal tissue is the key to developing new therapeutic radioimmunoconjugates.

One potential method for reducing off-target exposure is to remove the radioactivity more effectively from the body (e.g., from normal tissue in the body). One mechanism is to increase the rate of clearance of the biological targeting agent. This approach likely requires identifying ways to shorten the half-life of the biological targeting agent, which is not well described for biological targeting agents. Regardless of the mechanism, increasing drug clearance will also negatively impact the pharmacodynamics/efficacy in that the more rapid removal of drug from the body will lower the effective concentration at the site of action, which, in turn, would require a higher total dose and would not achieve the desired results of reducing total radioactive dose to normal tissue.

Other efforts have focused on accelerating the metabolism of the portion of the molecule that contains the radioactive moiety. To this end, some efforts have been made to increase the rate of cleavage of the radioactivity from the biological targeting agents using what have been termed “cleavable linkers”. Cleavable linkers, however, have been taken on different meaning as it relates to radioimmunoconjugates. Cornelissen, et al. has described cleavable linkers as those by which the bifunctional chelate attaches to the biologic targeting agent through a reduced cysteine, whereas others have described the use of enzyme-cleavable systems that require the co-administration of the radioimmunoconjugate with a cleaving agent/enzyme to release [Mol Cancer Ther. 2013, 12 (11), 2472-2482; Methods Mol Biol. 2009, 539, 191-211; Bioconjug Chem. 2003, 14 (5), 927-33]. These methods either change the nature of the biological targeting moiety, in the case of the cysteine linkage, or are not practical from a drug development perspective (enzyme cleavable systems) since, in the case of the citations provided, require the administration of two agents.

The present disclosure provides, among other things, compounds, e.g., radioimmunoconjugates, that are more effectively eliminated from the body after catabolism and/or metabolism, while maintaining therapeutic efficacy. Disclosed immunoconjugates may, in some embodiments, achieve a reduction of total body radioactivity, for example, by increasing the extent of excretion of the catabolic/metabolic products while maintaining the pharmacokinetics of the intact molecule when compared to known bifunctional chelates. In some embodiments, this reduction in radioactivity results from the clearance of catabolic/metabolic by-products without impacting other in vitro and in vivo properties such as binding specificity (in vitro binding), cellular retention, and tumor uptake in vivo. Thus, in some embodiments, provided compounds achieve reduced radioactivity in the human body while maintaining on-target activity.

### Definitions

As used herein, the term “bind” or “binding” of a targeting moiety means an at least temporary interaction or association with or to a target molecule, e.g., to human EGFRvIII and/or a mutational variant of EGFRvIII, e.g., as described herein.

The terms “bifunctional chelate,” as used herein, refers to a compound that comprises a chelate, a linker, and a cross-linking group. See. e.g., FIG. 1A. A “cross-linking group” is a reactive group that is capable of joining two or more molecules, e.g., joining a bifunctional chelate and a targeting moiety, by a covalent bond.

The term “bifunctional conjugate,” as used herein, refers to a compound that comprises a chelate or metal complex thereof, a linker, and a targeting moiety, e.g., an antibody or antigen-binding fragment thereof. See. e.g., FIG. 1B.

The term “cancer,” as used herein, refers to any cancer caused by the proliferation of malignant neoplastic cells, such as tumors, neoplasms, carcinomas, sarcomas, leukemias, and lymphomas. In some embodiments, a cancer of the present disclosure comprises cells (e.g., tumor cells) expressing EGFRVIII, such as, but not limited to, glioblastoma multiforme and carcinoma.

The term “chelate,” as used herein, refers to an organic compound or portion thereof that can be bonded to a central metal or radiometal atom at two or more points.

The term “conjugate,” as used herein, refers to a molecule that contains a chelating group or metal complex thereof, a linker group, and which optionally contains a targeting moiety, e.g., an antibody or antigen-binding fragment thereof.

As used herein, unless otherwise noted, the phrase “constant region,” when used in reference to an antibody or a fragment thereof (e.g., an IgG1, an IgG2, or an IgG4 constant region) is intended to encompass both wild type constant regions and variants (e.g., constant regions having at least 85%, at least 90%, at least 92%, at least 94%, at least 95%, at least 96%, at least 97%, at least 98%, or at least 99% amino acid sequence identity with a reference sequence for a wild-type constant region.

As used herein, the term “compound,” is meant to include all stereoisomers, geometric isomers, and tautomers of the structures depicted.

The compounds recited or described herein can be asymmetric (e.g., having one or more stereocenters). All stereoisomers, such as enantiomers and diastereomers, are intended unless otherwise indicated. Compounds discussed in the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis.

As used herein, “detection agent” refers to a molecule or atom which is useful in diagnosing a disease by locating the cells containing the antigen. Various methods of labeling polypeptides with detection agents are known in the art. Examples of detection agents include, but are not limited to, radioisotopes and radionuclides, dyes (such as with the biotin-streptavidin complex), contrast agents, luminescent agents (e.g., fluorescein isothiocyanate or FITC, rhodamine, lanthanide phosphors, cyanine, and near IR dyes), and magnetic agents, such as gadolinium chelates.

As used herein, the term “radionuclide,” refers to an atom capable of undergoing radioactive decay (e.g., ³H, ¹⁴C, ¹⁵N, ¹⁸F, ³⁵S, ⁴⁷Sc, ⁵⁵Co, ⁶⁰Cu, ⁶¹Cu, ⁶²Cu, ⁶⁴Cu, ⁶⁷Cu, ⁷⁵Br, ⁷⁶Br, ⁷⁷Br, ⁸⁹Zr, ⁸⁶Y, ⁸⁷Y, ⁹⁰Y, ⁹⁷Ru, ⁹⁹Tc, ^{99m}Tc, ¹⁰⁵Rh, ¹⁰⁹Pd, ¹¹¹In, ¹²³I, ¹²⁴I, ¹²⁵I, ¹³¹I, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰³Pb, ²¹¹At, ²¹²Pb, ²¹²Bi, ²¹³Bi, ²²³Ra, ²²⁵Ac, ²²⁷Th, ²²⁹Th, ⁶⁶Ga, ⁶⁷Ga, ⁶⁸Ga, ⁸²Rb, ^{117m}Sn, ²⁰¹Tl). The terms radioactive nuclide, radioisotope, or radioactive isotope may also be used to describe a radionuclide. Radionuclides may be used as detection agents, as described herein. In some embodiments, the radionuclide may be an alpha-emitting radionuclide.

The term an “effective amount” of an agent (e.g., any of the foregoing conjugates), as used herein, is that amount sufficient to effect beneficial or desired results, such as clinical results, and, as such, an “effective amount” depends upon the context in which it is being applied. For example, in therapeutic applications, an “effective amount” may be an amount sufficient to cure or at least partially arrest the symptoms of the disorder and its complications, and/or to substantially improve at least one symptom associated with the disease or a medical condition. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but may, for example, provide a treatment for a disease or condition such that the onset of the disease or condition is delayed, hindered, or prevented, such that the disease or condition symptoms are ameliorated, or such that the term of the disease or condition is changed. For example, the disease or condition may become less severe and/or recovery is accelerated in an individual. An effective amount may be administered by administering a single dose or multiple (e.g., at least two, at least three, at least four, at least five, or at least six) doses.

The term “immunoconjugate,” as used herein, refers to a conjugate that includes a targeting moiety, such as an antibody (or antigen-binding fragment thereof), nanobody, affibody, or a consensus sequence from Fibronectin type III domain. In some embodiments, the immunoconjugate comprises an average of at least 0.10 conjugates per targeting moiety (e.g., an average of at least 0.2, 0.3, 0.4, 0.5, 0.6, 0.7, 0.8, 0.9, 1, 2, 3, 4, 5, 6, 7, or 8 conjugates per targeting moiety).

The term “radioconjugate,” as used herein, refers to any conjugate that includes a radioisotope or radionuclide, such as any of the radioisotopes or radionuclides described herein.

The term “radioimmunoconjugate,” as used herein, refers to any immunoconjugate that includes a radioisotope or radionuclide, such as any of the radioisotopes or radionuclides described herein. A radioimmunoconjugate provided in the present disclosure typically refers to a bifunctional conjugate that comprises a metal complex formed from a radioisotope or radionuclide.

The term “radioimmunotherapy,” as used herein, refers a method of using a radioimmunoconjugate to produce a therapeutic effect. In some embodiments, radioimmunotherapy may include administration of a radioimmunoconjugate to a subject in need thereof, wherein administration of the radioimmunoconjugate produces a therapeutic effect in the subject. In some embodiments, radioimmunotherapy may include administration of a radioimmunoconjugate to a cell, wherein administration of the radioimmunoconjugate kills the cell. Wherein radioimmunotherapy involves the selective killing of a cell, in some embodiments the cell is a cancer cell in a subject having cancer.

The term “pharmaceutical composition,” as used herein, represents a composition containing a radioimmunoconjugate described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient.” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, radioprotectants, sorbents, suspending or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts of the compounds described here that are, within the scope of sound medical judgment, suitable for use in contact with the tissues of humans and animals without undue toxicity, irritation, or allergic response. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties. Selection. and Use, (Eds. P. H. Stahl and C. G. Wermuth), Wiley-VCH, 2008. Salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, among others. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “polypeptide,” as used herein, refers to a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means.

By “subject” is meant a human or non-human animal (e.g., a mammal).

By “substantial identity” or “substantially identical” is meant a polypeptide sequence that has the same polypeptide sequence, respectively, as a reference sequence, or has a specified percentage of amino acid residues, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). Sequence identity may be measured using sequence analysis software on the default setting (e.g., Sequence Analysis Software Package of the Genetics Computer Group, University of Wisconsin Biotechnology Center, 1710 University Avenue, Madison, WI 53705). Such software may match similar sequences by assigning degrees of homology to various substitutions, deletions, and other modifications.

As used herein, and as well understood in the art, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

As used herein, the term “about” or “approximately,” when used in reference to a quantitative value, includes the recited quantitative value itself, unless specifically stated otherwise. As used herein, the term “about” or “approximately” refers to a ±10% variation from the recited quantitative value unless otherwise indicated or inferred from the context.

As used herein, the term “targeting moiety” refers to any molecule or any part of a molecule that is capable of binding to a given target. The term, “EGFRvIII targeting moiety” refers to a targeting moiety that is capable of binding to an EGFRVIII molecule, e.g., a human EGFRvIII.

As used herein, unless otherwise specified, “EGFR” refers to human EGFR. Human EGFR means a full-length protein defied by UniProt P00533, or a fragment or variant thereof. EGFR is also known as epidermal growth factor receptor, ErbB-1, and HER1. In some particular embodiments, EGFR of non-human species, e.g., mouse EGFR, is used. The terms “wt EGFR”, “WT EGFR”, “EGFR WT,” and “EGFR wt” are used interchangeably and refer to wild type EGFR.

As used herein, “EGFRvIII” or “vIII” refers to an EGFR variant resulting from in-frame deletion of exons 2-7 of the coding sequence, or a fragment thereof. EGFRvIII is also known as epidermal growth factor receptor variant III, de2-7EGFR, and ΔEGFR.

As used herein, the term “fragment,” when used to refer to an EGFRVIII fragment, refers to N-terminally and/or C-terminally truncated EGFRvIII or protein domains of EGFRvIII. Unless otherwise noted, fragments of EGFRVIII used in accordance with embodiments described herein retain the capability of the full-length EGFRVIII to be recognized and/or bound by an EGFRvIII-targeting moiety as described in the present disclosure. As an illustrative example, the fragment may be an extracellular domain of EGFRvIII, such as amino acid residues 1 to 76 of EGFRVIII (SEQ ID NO: 119), amino acid residues 1 to 18 of EGFRVIII (SEQ ID NO:125), or amino acid residues 15 to 37 of EGFRVIII (SEQ ID NO:6).

The use of the terms “a” and “an” and “the” and similar referents in the context of describing the disclosure (especially in the context of the claims) are to be construed to cover both the singular and the plural, unless otherwise indicated herein or clearly contradicted by context.

Unless specifically stated or obvious from context, as used herein the term “or” is understood to be inclusive and covers both “or” and “and”.

The term “and/or” where used herein is to be taken as specific disclosure of each of the specified features or components with or without the other.

The terms “comprising”, “having”, “including”, and “containing” are to be construed as open-ended terms (i.e., meaning “including, but not limited to”) unless otherwise noted. The term “consisting of” is to be construed as close-ended.

As used herein, the term “native” with respect to a protein such as EGFRvIII or EGFR refers to the natural conformation of the protein and includes proteins that are properly folded and/or functional.

As used herein, the term “denatured” with respect to a protein such EGFRvIII or EGFR refers to a protein that has lost its natural conformation and may entail, for example, a loss in the tertiary and secondary structure.

As used herein, the expression “a peptide comprising or consisting of an EGFRvIII fragment” means that the peptide may comprise a portion other than the EGFRVIII fragment or that it consists of the EGFRVIII fragment.

As used herein, a targeting moiety (e.g., an antibody or antigen-binding domain) “binds to an epitope comprising amino acid residues” means that said amino acid residues are either part of the epitope or that it is necessary for the binding of the targeting moiety.

As used herein, when used in reference to a targeting moiety (e.g., an antibody or antigen-binding domain), the term “fails to bind to” a peptide or protein means that the targeting moiety (e.g., antibody or antigen binding fragment) a) does not bind significantly to the peptide or protein when expressed recombinantly or in cells, b) does not bind to the peptide or protein with detectable affinity, c) has similar binding property as a negative control molecule, d) does not bind specifically to the peptide or protein, or e) binds with a value between 0% and 15% as determined by flow cytometry experiments known in the field.

As used herein the term “autologous” refers to materials derived from the same individual.

As used herein, the term “antigen-binding domain” refers to the domain of an antibody or of an antigen-binding fragment which allows specific binding to an antigen.

As used herein, the term “antibody” encompasses monoclonal antibody, polyclonal antibody, humanized antibody, chimeric antibody, human antibody, single domain antibody (such as a VHH, VH, VL, nanobody, or any camelid or llama single domain antibody), multispecific antibody (e.g., bispecific antibodies) etc. The term “antibody” encompasses molecules that have a format similar to those occurring in nature (e.g., human IgGs, etc.). The term “antibody”, also referred to in the art as “immunoglobulin” (Ig), as used herein refers to a protein constructed from paired heavy and light polypeptide chains; various Ig isotypes exist, including IgA. IgD. IgE. IgG, and IgM. When an antibody is correctly folded, each chain folds into a number of distinct globular domains joined by more linear polypeptide sequences. For example, the immunoglobulin light chain folds into a variable (VL) and a constant (CL) domain, while the heavy chain folds into a variable (VH) and three constant (CH₁. CH₂. CH₃) domains. Interaction of the heavy and light chain variable domains (VH and VL) results in the formation of an antigen-binding region (Fv). Each domain has a well-established structure familiar to those of skill in the art.

Typically, an antibody is constituted from the pairing of two light chains and two heavy chains. Different antibody isotypes exist, including IgA, IgD, IgE, IgG and IgM. Human IgGs are further divided into four distinct sub-groups namely; IgG1, IgG2, IgG3 and IgG4. Therapeutic antibodies are generally developed as IgG1 or IgG2 or lgG4.

In an exemplary embodiment, the antibody or antigen-binding fragment of the present disclosure may comprise, for example, a human IgG1 constant region or a fragment thereof. In another exemplary embodiment, the antibody or antigen-binding fragment of the present disclosure may comprise, for example, a human IgG2 constant region or a fragment thereof. In another exemplary embodiment, the antibody or antigen-binding fragment of the present disclosure may comprise, for example, a human IgG4 constant region or a fragment or thereof. Constant regions of other antibody subtypes are also contemplated.

The light chain and heavy chain of human antibody IgG isotypes each comprise a variable region having 3 hypervariable regions named complementarity determining regions (CDRs). The light chain CDRs are identified herein as CDRL1 or L1. CDRL2 or L² and CDRL3 or L³. The heavy chain CDRs are identified herein as CDRH1 or H1. CDRH2 or H2 and CDRH3 or H3. Complementarity determining regions are flanked by framework regions (FR) in the order: FR1-CDR1-FR2-CDR2-FR3-CDR3-FR4. The light and heavy chain variable regions are responsible for binding the target antigen and can therefore show significant sequence diversity between antibodies. The constant regions show less sequence diversity and are responsible for binding a number of natural proteins to elicit important biochemical events. The variable region of an antibody contains the antigen-binding determinants of the molecule, and thus determines the specificity of an antibody for its target antigen. The majority of sequence variability occurs in the CDRs which combine to form the antigen-binding site and contribute to binding and recognition of an antigenic determinant. The framework regions may play a role in the proper positioning and alignment in three dimensions of the CDRs for optimal antigen-binding. The specificity and affinity of an antibody for its antigen is determined by the structure of the hypervariable regions, as well as their size, shape, and chemistry of the surface they present to the antigen. Various schemes exist for identification of the regions of hypervariability, the two most common being those of Kabat and of Chothia and Lesk. Kabat et al (1991) define the “complementarity-determining regions” (CDR) based on sequence variability at the antigen-binding regions of the VH and VL domains. Chothia and Lesk (1987) define the “hypervariable loops” (H or L) based on the location of the structural loop regions in the VH and VL domains. These individual schemes define CDR and hypervariable loop regions that are adjacent or overlapping, those of skill in the antibody art often utilize the terms “CDR” and “hypervariable loop” interchangeably, and they may be so used herein. The CDR/loops are identified herein according to the Kabat scheme except the CDRH1 loops that is delineated by combining the Kabat and Chothia definitions.

Recombinant DNA technology now allows the design of various antibody format such as single chain antibodies (e.g., single domain), diabody, minibody, nanobody and the like which are encompassed by the present disclosure.

An “antigen-binding fragment,” as referred to herein, may include any suitable antigen-binding fragment known in the art. The antigen-binding fragment may be a naturally-occurring fragment or may be obtained by manipulation of a naturally-occurring antibody or by using recombinant methods. For example, an antibody fragment may include, but is not limited to a Fv, single-chain Fv (scFv; a molecule consisting of VL and VH connected with a peptide linker), Fab, F(ab) 2, single-domain antibody (sdAb; a fragment composed of a single VL or VH), and multivalent presentations of any of these. Antibody fragments such as those just described may require linker sequences, disulfide bonds, or other type of covalent bond to link different portions of the fragments; those of skill in the art will be familiar with the requirements of the different types of fragments and various approaches and various approaches for their construction.

Antigen-binding fragments thereof of the present disclosure encompass molecules having an antigen-binding site comprising amino acid residues that confer specific binding to an antigen (e.g., one or more CDRs).

Exemplary embodiments of antigen-binding fragments disclosure thus includes without limitation (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR), e.g., V_{H} CDR3.

Specific embodiments of antigen-binding fragments may include for example, a scFv, a Fab, a Fab′ or a (Fab′)₂.

The term “humanized antibody” encompasses fully humanized antibody (i.e., frameworks are 100% humanized) and partially humanized antibody (e.g., at least one variable region contains one or more amino acids from a human antibody, while other amino acids are amino acids of a non-human parent antibody). Typically, a “humanized antibody” contains CDRs of a non-human parent antibody (e.g., mouse, rat, rabbit, non-human primate, etc.) and frameworks that are identical to those of a natural human antibody or of a human antibody consensus. In such instance, those “humanized antibodies” are characterized as fully humanized. A “humanized antibody” may also contain one or more amino acid substitutions that have no correspondence to those of the human antibody or human antibody consensus. Such substitutions include, for example, back-mutations (e.g., re-introduction of non-human amino acids) that may preserve the antibody characteristics (e.g., affinity, specificity etc.). Such substitutions are usually in the framework region. A “humanized antibody” usually also comprises a constant region (Fc) which is typically that of a human antibody. Typically, the constant region of a “humanized antibody” is identical to that of a human antibody. A humanized antibody may be obtained by CDR grafting (Tsurushita et al, 2005; Jones et al, 1986; Tempest et al, 1991; Riechmann et al, 1988; Queen et al, 1989). Such antibody is considered as fully humanized.

The term “chimeric antibody” refers to an antibody having a constant region from an origin distinct from that of the parent antibody. The term “chimeric antibody” encompasses antibodies having a human constant region. Typically, a “chimeric antibody” is composed of variable regions originating from a mouse antibody and of a human constant region.

The term “hybrid antibody” refers to an antibody comprising one of its heavy or light chain variable region (its heavy or light chain) from a certain type of antibody (e.g., humanized) while the other of the heavy or light chain variable region (the heavy or light chain) is from another type (e.g., murine, chimeric).

Antibodies and/or antigen-binding fragments of the present disclosure may originate, for example, from a mouse, a rat or any other mammal or from other sources such as through recombinant DNA technologies. Antibodies or antigen-binding fragment of the present disclosure may include for example, a synthetic antibody, a non-naturally occurring antibody, an antibody obtained following immunization of a non-human mammal etc.

Antibodies or antigen-binding fragments thereof of the present disclosure may be isolated and/or substantially purified.

### Compounds, e.g., Radioimmunoconjugates

In one aspect, this disclosure provides compounds, e.g., radioimmunoconjugates, comprising the following structure, or pharmaceutically acceptable salts thereof:

A-L¹-(L²)ₙ-B Formula I

wherein
A is a chelating moiety or a metal complex thereof,
B is a targeting moiety that is capable of binding to epidermal growth factor receptor variant III (EGFRvIII), wherein EGFRvIII comprises a peptide consisting of amino acid residues 1 to 76 of EGFRvIII (SEQ ID NO: 119);
L′ is a bond, C—O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
L² each independently has the structure of Formula II:

—X¹-L³-Z¹— Formula II

wherein
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—*, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—*, or —NR¹—*; wherein “*” indicates the attachment point to L³, and R¹ is hydrogen, optionally substituted C₁-C₆ alkyl, optionally substituted C₁-C₆ heteroalkyl, or optionally substituted aryl or heteroaryl;
L³ is optionally substituted C₁-C₅₀ alkyl or optionally substituted C₁-C₅₀ heteroalkyl; and
Z¹ is —CH₂-#, —C(O)-#, —C(S)-#, —OC(O)-#, —C(O)O-#, —NR²C(O)-#, —C(O)NR²-#, or —NR²-#, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl.

Typical substituents of alkyl, heteroalkyl, aryl, or heteroaryl include, but are not limited to halo (e.g., F, Cl, Br, I), OH, CN, nitro, amino, C₁-6 alkyl, C₂-6 alkenyl, C₂-6 alkynyl, C₃-8 cycloalkyl, C₁-6 heteroalkyl, C₁-6 heterocycloalkyl, haloalkyl (e.g., CF₃), alkoxy (e.g., OCH₃), alkylamino (e.g., NH₂CH₃), sulfonyl, aryl, and heteroaryl.

In some embodiments, the compound has or comprises the structure shown below:

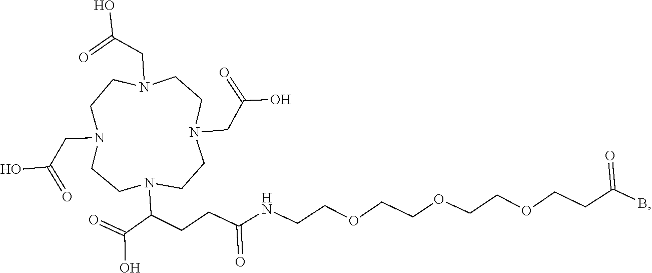

wherein B is an EGFRvIII targeting moiety (e.g., an EGFRvIII antibody or antigen-binding fragment thereof).

In some embodiments, A-L-comprises one of the following structures, or a metal complex thereof:

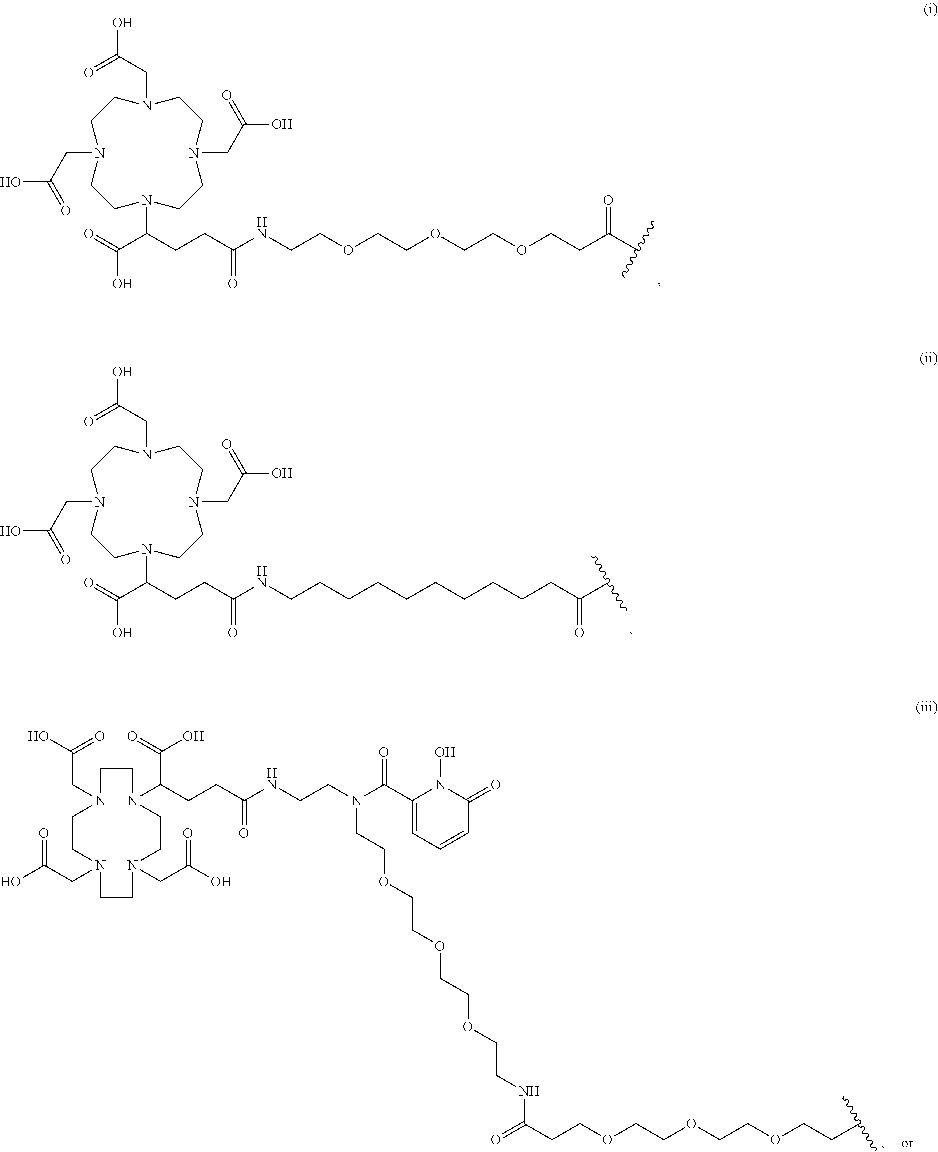 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCNC(=O)CCC(C(=O)O)N1CCN(CC(=O)O)CCN(CC(=O)O)CCN(CC(=O)O)CC1 ; *CCOCCOCCOCCC(=O)NCCOCCOCCOCCN(CCNC(=O)CCC(C(=O)O)N1CCN(CC(=O)O)CCN(CC(=O)O)CCN(CC(=O)O)CC1)C(=O)c1cccc(=O)n1O]

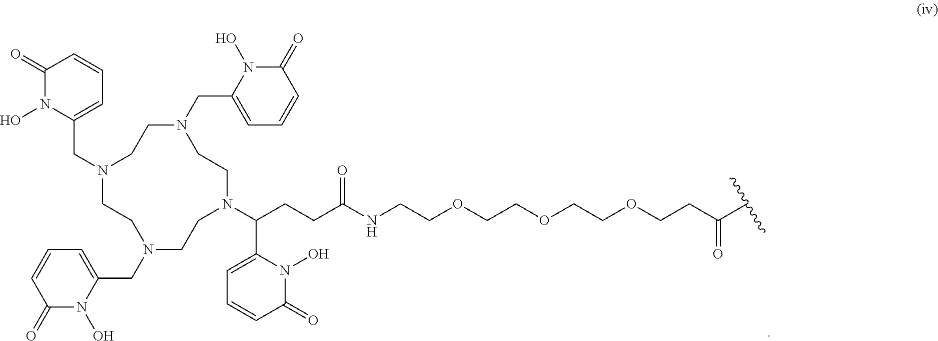 [SMILES: *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

In some embodiments, as further described herein, the compound (e.g., radioimmunoconjugate) comprises a chelating moiety or a metal complex thereof, which metal complex may comprise a radionuclide. In some such compounds, the average ratio or median ratio of the chelating moiety to the EGFRVIII targeting moiety is eight or less, seven or less, six or less, five or less, four or less, three or less, two or less, or about one. In some compounds, the average ratio or median ratio of the chelating moiety to the EGFRvIII targeting moiety is about one.

In some embodiments, after a radioimmunoconjugate is administered to a mammal, the proportion of radiation (of the total amount of radiation that is administered) that is excreted by the intestinal route, the renal route, or both is greater than the proportion of radiation excreted by a comparable mammal that has been administered a reference radioimmunoconjugate. By “reference immunoconjugate” it is meant a known radioimmunoconjugate that differs from a radioimmunoconjugate described herein at least by (1) having a different linker; (2) having a targeting moiety of a different size and/or (3) lacking a targeting moiety. In some embodiments, the reference radioimmunoconjugate is selected from the group consisting of [Y]-ibritumomab tiuxetan (Zevalin (⁹⁰Y)) and [¹¹¹In]-ibritumomab tiuxetan (Zevalin (¹¹¹In)).

In some embodiments, the proportion of radiation excreted by a given route or set of routes) is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater than the proportion of radiation excreted by the same route(s) by a comparable mammal that has been administered a reference radioimmunoconjugate. In some embodiments, the proportion of radiation excreted is at least 1.5-fold, at least 2-fold, at least 2.5-fold, at least 3-fold, at least 3.5-fold, at least 4-fold, at least 4.5 fold, at least 5 fold, at least 6-fold, at least 7-fold, at least 8-fold, at least 9-fold, or at least 10-fold greater than proportion of radiation excreted by a comparable mammal that has been administered a reference radioimmunoconjugate. The extent of excretion can be measured by methods known in the art, e.g., by measuring radioactivity in urine and/or feces and/or by measuring total body radioactivity over a period time. See also, e.g., International Patent Publication WO 2018/024869.

In some embodiments, the extent of excretion is measured at a time period of at least or about 12 hours after administration, at least or about 24 hours after administration, at least or about 2 days after administration, at least or about 3 days after administration, at least or about 4 days after administration, at least or about 5 days after administration, at least or about 6 days after administration, or at least or about 7 days, after administration.

In some embodiments, after a compound (e.g., radioimmunoconjugate) has been administered to a mammal, the compound (e.g., radioimmunoconjugate) exhibits decreased off-target binding effects (e.g., toxicities) as compared to a reference compound (e.g., reference conjugate, e.g., a reference immunoconjugate such as a reference radioimmunoconjugate). In some embodiments, this decreased off-target binding effect is a feature of a compound (e.g., radioimmunoconjugate) that also exhibits a greater excretion rate as described herein.

### Targeting Moieties

Targeting moieties include any molecule or any part of a molecule that is capable of binding (e.g., capable of specifically binding, specifically binds to, etc.) to a given target, e.g., EGFRvIII. In some embodiments, the targeting moiety comprises a protein or polypeptide. In some embodiments, the targeting moiety is selected from the group consisting of antibodies or antigen binding fragments thereof, nanobodies, affibodies, and consensus sequences from Fibronectin type III domains (e.g., Centyrins or Adnectins). In some embodiments, a moiety is both a targeting and a therapeutic moiety, i.e., the moiety is capable of binding to a given target and also confers a therapeutic benefit. In some embodiments, the targeting moiety comprises a small molecule.

In some embodiments, the targeting moiety has a molecular weight of at least 50 kDa, at least 75 kDa, at least 100 kDa, at least 125 kDa, at least 150 kDa, at least 175 kDa, at least 200 kDa, at least 225 kDa, at least 250 kDa, at least 275 kDa, or at least 300 kDa.

Typically, the targeting moiety is capable of binding to EGFRVIII, or a fragment thereof. In some embodiments, the targeting moiety is capable of binding to human EGFRVIII, or a fragment thereof.

In some embodiments, the targeting moiety is capable of binding specifically to EGFRvIII (e.g., is capable of binding to EGFRVIII while exhibiting comparatively little or no binding to wt EGFR).

In some embodiments, the targeting moiety is capable of binding to an extracellular region of EGFRvIII, e.g., domain III (L²) of EGFRVIII, domain IV (CRII) of EGFRvIII, amino acid residues 1 to 76 of EGFRVIII (SEQ ID NO:119), amino acid residues 1 to 18 of EGFRVIII (SEQ ID NO: 125), or amino acid residues 15 to 37 of EGFRVIII (SEQ ID NO: 6).

In some embodiments, the targeting moiety does not bind to wild type EGFR with detectable affinity. By “detectable affinity,” it is generally meant that the binding ability between a targeting moiety and its target, reported by a K_{D}, EC₅₀, or IC₅₀ value, is at most about 10⁵ M or lower. A binding affinity, reported by a K_{D}, EC₅₀, or IC₅₀ value, higher than 10⁵ M is generally no longer measurable with common methods such as ELISA and flow cytometry, and is, therefore, of secondary importance.

In some embodiments, the targeting moiety inhibits EGFRVIII. By “inhibits,” it is meant that the targeting moiety at least partially inhibits one or more functions of EGFRvIII (e.g., human EGFRvIII). In some embodiments, the targeting moiety impairs signaling downstream of EGFRvIII, e.g., results in the suppressed growth of EGFRvIII-positive tumor cells. In some embodiments, the targeting moiety blocks ligand binding to EGFRvIII and/or receptor dimerization of EGFRvIII.

Generally, a targeting moiety of the present disclosure may be able to bind to a peptide comprising an EGFRVIII fragment consisting of amino acid residues 1 to 76 of EGFRVIII (SEQ ID NO:119). In some embodiments, the targeting moiety is able to bind to amino acid residues 1 to 18 of EGFRVIII (SEQ ID NO:125). In some other embodiments, the targeting moiety is able to bind amino acid residues 15 to 37 of EGFRVIII (SEQ ID NO:6).

Embodiments of EGFRvIII targeting moieties encompassed by the present disclosure includes, for example:
EGFRVIII targeting moieties that are able to bind to a peptide comprising an EGFRVIII fragment consisting of amino acid residues 1 to 18 of EGFRVIII (SEQ ID NO:125);
EGFRVIII targeting moieties that are able to bind to a peptide comprising an EGFRvIII fragment consisting of amino acid residues 3 to 18 of EGFRVIII (SEQ ID NO:129);
EGFRVIII targeting moieties that are able to bind to a peptide comprising an EGFRvIII fragment consisting of amino acid residues 15 to 37 of EGFRVIII (SEQ ID NO:6); or
EGFRVIII targeting moieties that are able to bind to a peptide comprising an EGFRVIII fragment consisting of amino acid residues 19 to 37 of EGFRVIII (SEQ ID NO: 142).

Some particular EGFRVIII targeting moieties (e.g., EGFRVIII antibodies or antigen-binding fragments thereof) encompassed by the present disclosure include those that do not require the presence of amino acid residues 1-2 of EGFRvIIII for binding. Particularly contemplated are EGFRVIII targeting moieties (e.g., EGFRvIII antibodies or antigen-binding fragments thereof) that are capable of binding to one or more EGFRVIII fragments of amino acid residues 19-76 (SEQ ID NO: 138), amino acid residues 19-62 (SEQ ID NO:139), amino acid residues 19-49 (SEQ ID NO:140), amino acid residues 19-45 (SEQ ID NO: 141), amino acid residues 28-45 (SEQ ID NO:143), amino acid residues 28-37 (SEQ ID NO:144), amino acid residues 19-37 (SEQ ID NO:142), amino acid residues 3-45 (SEQ ID NO: 127), amino acid residues 3-49 (SEQ ID NO:126), amino acid residues 3-37 (SEQ ID NO: 128), amino acid residues 6-49 (SEQ ID NO:130), amino acid residues 6-45 (SEQ ID NO:131), amino acid residues 6-37 (SEQ ID NO:132), amino acid residues 10-49 (SEQ ID NO:133), amino acid residues 10-45 (SEQ ID NO:134), amino acid residues 10-37 (SEQ ID NO:135), amino acid residues 15-49 (SEQ ID NO:136), amino acid residues 15-45 (SEQ ID NO:137), or amino acid residues 15-37 (SEQ ID NO:6) of EGFRvIII.

In some embodiments, EGFRvIII targeting moieties (e.g., EGFRvIII antibodies or antigen-binding fragments thereof) provided herein and/or used in accordance with the present disclosure are able to bind to a peptide comprising an EGFRVIII fragment consisting of amino acid residues 3 to 37 of EGFRVIII (SEQ ID NO: 128), such as F260-5G6 (referred herein also as 5G6), F263-1A8 (referred herein also as 1A8), F263-4B3 (referred herein also as 4B3), F263-4E11 (referred herein also as 4E11), F263-5D8 (referred herein also as 5D8) and F265-9C₉ (referred to herein also as 9C₉) antibody.

In some embodiments, EGFRvIII targeting moieties (e.g., EGFRvIII antibodies or antigen-binding fragments) provided herein are able to bind to a peptide comprising an EGFRvIII fragment consisting of amino acid residues 1 to 33 of EGFRVIII (SEQ ID NO: 124).

In some embodiments, EGFRvIII targeting moieties (e.g., EGFRvIII antibodies or antigen-binding fragments thereof) of the present specifically bind to EGFRVIII (SEQ ID NO: 5) and are capable of binding to an EGFRVIII fragment selected from the group consisting of:
a. a fragment consisting of amino acid residues 15 to 37 of EGFRVIII (SEQ ID NO:6);
b. a fragment consisting of amino acid residues 1 to 76 of EGFRVIII (SEQ ID NO:119);
c. a fragment consisting of amino acid residues 1 to 62 of EGFRVIII (SEQ ID NO: 120);
d. a fragment consisting of amino acid residues 1 to 49 of EGFRVIII (SEQ ID NO:121);
e. a fragment consisting of amino acid residues 1 to 45 of EGFRVIII (SEQ ID NO:122);
f. a fragment consisting of amino acid residues 1 to 37 of EGFRVIII (SEQ ID NO:123);
g. a fragment consisting of amino acid residues 3 to 49 of EGFRVIII (SEQ ID NO:126);
h. a fragment consisting of amino acid residues 3 to 45 of EGFRVIII (SEQ ID NO:127);
i. a fragment consisting of amino acid residues 3 to 37 of EGFRVIII (SEQ ID NO: 128);
j. a fragment consisting of amino acid residues 6 to 49 of EGFRVIII (SEQ ID NO:130);
k. a fragment consisting of amino acid residues 6 to 45 of EGFRVIII (SEQ ID NO:131);
l. a fragment consisting of amino acid residues 6 to 37 of EGFRVIII (SEQ ID NO:132);
m. a fragment consisting of amino acid residues 10 to 49 of EGFRVIII (SEQ ID NO:133);
n. a fragment consisting of amino acid residues 10 to 45 of EGFRvIII (SEQ ID NO:134);
o. a fragment consisting of amino acid residues 10 to 37 of EGFRVIII (SEQ ID NO:135);
p. a fragment consisting of amino acid residues 15 to 49 of EGFRVIII (SEQ ID NO:136);
q. A fragment consisting of amino acid residues 15 to 45 of EGFRVIII (SEQ ID NO: 137);
r. a fragment consisting of amino acid residues 19 to 76 of EGFRvIII (SEQ ID NO:138);
s. a fragment consisting of amino acid residues 19 to 62 of EGFRVIII (SEQ ID NO: 139);
t. a fragment consisting of amino acid residues 19 to 49 of EGFRVIII (SEQ ID NO: 140);
u. a fragment consisting of amino acid residues 19 to 45 of EGFRvIII (SEQ ID NO:141);
v. a fragment consisting of amino acid residues 19 to 37 of EGFRVIII (SEQ ID NO: 142); and
w. any combination of the above fragments thereof,
   wherein the EGFRvIII targeting moiety fails to bind a peptide comprising or consisting of the amino acid sequence set forth in SEQ ID NO: 149.

In some embodiments, the targeting moiety (e.g., antibody or antigen-binding fragment thereof) of the present disclosure may be capable of binding to a peptide comprising or consisting of an amino acid sequence selected from the group consisting of SEQ ID NO: 145, SEQ ID NO: 146, SEQ ID NO: 147, SEQ ID NO: 148, SEQ ID NO: 150, SEQ ID NO: 151, SEQ ID NO: 152, SEQ ID NO: 153, SEQ ID NO: 155, SEQ ID NO: 156, SEQ ID NO: 157, SEQ ID NO: 162, SEQ ID NO: 164, SEQ ID NO: 165, and combination thereof.

In some embodiments, the targeting moiety (e.g., antibody or antigen-binding fragment thereof) may be capable of binding to a peptide comprising or consisting of an amino acid sequence set forth in SEQ ID NO:160.

In some embodiments, provided herein are targeting moieties (e.g., antibodies or antigen-binding fragments thereof) that specifically bind to EGFRVIII (SEQ ID NO:5) and that are capable of binding to an EGFRvIII fragment selected from the group consisting of:
a. a fragment consisting of amino acid residues 15 to 37 of EGFRVIII (SEQ ID NO:6);
b. a fragment consisting of amino acid residues 1 to 76 of EGFRvIII (SEQ ID NO:119);
c. a fragment consisting of amino acid residues 1 to 49 of EGFRVIII (SEQ ID NO: 121);
d. a fragment consisting of amino acid residues 1 to 37 of EGFRvIII (SEQ ID NO:123);
e. a fragment consisting of amino acid residues 3 to 37 of EGFRVIII (SEQ ID NO:128); and
f. any combination of the above fragments thereof,
   wherein the targeting moieties fail to bind a peptide comprising of consisting of the amino acid sequence set forth in SEQ ID NO: 149.

In some embodiments, the targeting moiety (e.g., antibody or antigen-binding fragment thereof) of the present disclosure may be capable of binding to a peptide comprising or consisting of an amino acid sequence selected from the group consisting of SEQ ID NO: 145, SEQ ID NO: 146, SEQ ID NO: 147, SEQ ID NO: 151, SEQ ID NO: 152, SEQ ID NO: 153, SEQ ID NO: 155, SEQ ID NO: 156, SEQ ID NO: 157, SEQ ID NO: 158, SEQ ID NO: 160, SEQ ID NO: 161, SEQ ID NO: 162, SEQ ID NO: 164, SEQ ID NO: 165, and combination thereof. In some embodiments, the targeting moiety (e.g., antibody or antigen-binding fragment thereof) may be capable of binding to a peptide comprising or consisting of an amino acid sequence set forth in SEQ ID NO:154. In some embodiments, the targeting moiety (e.g., antibody or antigen-binding fragment thereof) may be capable of binding to a peptide comprising or consisting of an amino acid sequence set forth in SEQ ID NO: 159.

In some other embodiments, provided targeting moieties (e.g., antibodies or antigen-binding fragments thereof) specifically bind to EGFRvIII (SEQ ID NO:5) and are capable of binding to an EGFRVIII fragment selected from the group consisting of:
a. a fragment consisting of amino acid residues 15 to 37 of EGFRVIII (SEQ ID NO:6);
b. a fragment consisting of amino acid residues 1 to 76 of EGFRvIII (SEQ ID NO:119);
c. a fragment consisting of amino acid residues 1 to 62 of EGFRVIII (SEQ ID NO: 120);
d. a fragment consisting of amino acid residues 1 to 49 of EGFRVIII (SEQ ID NO:121);
e. a fragment consisting of amino acid residues 1 to 45 of EGFRVIII (SEQ ID NO: 122);
f. a fragment consisting of amino acid residues 1 to 37 of EGFRvIII (SEQ ID NO:123);
g. a fragment consisting of amino acid residues 3 to 49 of EGFRVIII (SEQ ID NO:126);
h. a fragment consisting of amino acid residues 3 to 45 of EGFRvIII (SEQ ID NO:127);
i. a fragment consisting of amino acid residues 3 to 37 of EGFRVIII (SEQ ID NO: 128);
j. a fragment consisting of amino acid residues 6 to 49 of EGFRVIII (SEQ ID NO: 130);
k. a fragment consisting of amino acid residues 6 to 45 of EGFRvIII (SEQ ID NO:131);
l. a fragment consisting of amino acid residues 6 to 37 of EGFRvIII (SEQ ID NO:132);
m. a fragment consisting of amino acid residues 10 to 49 of EGFRVIII (SEQ ID NO:133);
n. a fragment consisting of amino acid residues 10 to 45 of EGFRvIII (SEQ ID NO: 134);
o. a fragment consisting of amino acid residues 10 to 37 of EGFRvIII (SEQ ID NO:135);
p. a fragment consisting of amino acid residues 15 to 49 of EGFRVIII (SEQ ID NO:136);
q. a fragment consisting of amino acid residues 15 to 45 of EGFRVIII (SEQ ID NO:137); and
r. any combination of the above fragments thereof.

In some embodiments, the targeting moieties (e.g., antibodies or antigen-binding fragments thereof) may bind to;
a. a fragment consisting of amino acid residues 19 to 49 of EGFRVIII (SEQ ID NO:140);
b. a fragment consisting of amino acid residues 19 to 37 of EGFRVIII (SEQ ID NO:142);
c. a fragment consisting of amino acid residues 28 to 45 of EGFRVIII (SEQ ID NO: 143);
d. a fragment consisting of amino acid residues 28 to 37 of EGFRvIII (SEQ ID NO: 144);
   or any combination of the above fragments thereof.

In some embodiments, the targeting moiety (e.g., antibody or antigen-binding fragment thereof) may be capable of binding to a peptide comprising or consisting of an amino acid sequence selected from the group consisting SEQ ID NO: 145, SEQ ID NO: 147, SEQ ID NO: 148, SEQ ID NO:150, SEQ ID NO:151, SEQ ID NO:152, SEQ ID NO:153, SEQ ID NO:155, SEQ ID NO: 156, SEQ ID NO: 157, SEQ ID NO: 165, and combination thereof. In some embodiments, the targeting moiety (e.g., antibody or antigen-binding fragment thereof) may be capable of binding to a peptide comprising or consisting of an amino acid sequence set forth in SEQ ID NO:149.

Also provided are EGFRVIII targeting moieties (e.g., EGFRvIII antibodies or antigen-binding fragments thereof) that are able to bind an epitope comprising or involving amino acid residue Cys20 in said peptide. These include, for example, EGFRvIII antibodies or antigen-binding fragments thereof that bind EGFRvIII and/or a peptide comprising an EGFRvIII fragment consisting of the amino acid sequence set forth in SEQ ID NO:6 but are not able to bind a peptide comprising or consisting of the amino acid sequence SCVRAAGADSYEMEEDGVRKCKK (SEQ ID NO:149). Such antibodies or antigen binding fragments thereof encompass, for example, the 4B3, 5D8, and 4E11 antibodies.

Also specifically encompassed by the present disclosure are EGFRVIII targeting moieties (e.g., EGFRvIII antibodies or antigen-binding fragments thereof) that are able to bind an epitope comprising or involving amino acid residue Cys35 in said peptide. These include, for example, EGFRvIII antibodies or antigen-binding fragments thereof that bind EGFRvIII and/or a peptide comprising an EGFRVIII fragment consisting of the amino acid sequence set forth in SEQ ID NO:6 but are not able to bind a peptide comprising or consisting of the amino acid sequence SCVRACGADSYEMEEDGVRKAKK (SEQ ID NO: 163). Such antibodies or antigen binding fragments thereof encompass, for example, the 4B3, 5D8, 9C₉ and 4E11 antibodies.

Further encompassed by the present disclosure are EGFRVIII targeting moieties (e.g., EGFRVIII antibodies or antigen-binding fragments thereof) that are able to bind an epitope in a peptide comprising or involving amino acid residue Cys20 and Cys35 in said peptide. These include, for example, EGFRvIII antibodies or antigen-binding fragments thereof that bind EGFRvIII and/or a peptide comprising or consisting of an EGFRvIII fragment set forth in SEQ ID NO:6 but are not able to bind a peptide comprising or consisting of the amino acid sequence selected from SCVRAAGADSYEMEEDGVRKCKK (SEQ ID NO:149) or SCVRACGADSYEMEEDGVRKAKK (SEQ ID NO: 163). Such antibodies or antigen binding fragments thereof encompass, for example, the 4B3, 5D8 and 4E11 antibodies.

In some embodiments, in addition to amino acid residues Cys20 and/or Cys35, the epitope to which the EGFRVIII targeting moieties (e.g., EGFRvIII antibodies or antigen-binding fragments thereof) of the present disclosure bind or which are involved in their binding may further include amino acid residues Glu26, Asp30, Gly31, and/or Arg33. In some embodiments, the epitope may further include Asp23 and/or Val32.

For example, in some embodiments, EGFRvIII targeting moieties (e.g., EGFRvIII antibodies or antigen-binding fragments thereof) of the present disclosure bind to an epitope comprising or involving:
Cys20, Glu26, Asp30, Gly31, Arg33, and Cys35; or
Cys20, Asp23, Glu26, Asp30, Gly31, Val32, Arg33, and Cys35.

In some embodiments, in addition to amino acid residues Cys20 and/or Cys35, the epitope to which the EGFRVIII targeting moieties (e.g., EGFRVIII antibodies or antigen-binding fragments thereof) bind or involved in their binding may further include Arg18 and/or Gly21. In some embodiments, the epitope may further include Glu26 and/or Gly31.

For example, in some embodiments, EGFRvIII targeting moieties (e.g., EGFRvIII antibodies or antigen-binding fragments thereof) of the present disclosure bind to an epitope comprising or involving:
Arg18, Cys20, Gly21 and Cys35; or
Arg18, Cys20, Gly21, Glu26, Gly31 and Cys35.

In some other embodiments, EGFRvIII targeting moieties (e.g., EGFRvIII antibodies or antigen-binding fragments thereof) of the present disclosure bind to an epitope comprising or involving:
Cys16, Glu26, Gly31, Val32, Arg33, Lys34, Cys35 and Lys36; or
Cys16, Cys20, Glu26, Asp30, Gly31, Val32, Arg33, Lys34, Cys35 and Lys36.

### Antibodies and Antigen-Binding Moieties

Antibodies typically comprise two identical light polypeptide chains and two identical heavy polypeptide chains linked together by disulfide bonds. The first domain located at the amino terminus of each chain is variable in amino acid sequence, providing the antibody-binding specificities of each individual antibody. These are known as variable heavy (VH) and variable light (VL) regions. The other domains of each chain are relatively invariant in amino acid sequence and are known as constant heavy (CH) and constant light (CL) regions. Light chains typically comprise one variable region (VL) and one constant region (CL). An IgG heavy chain includes a variable region (VH), a first constant region (CHI), a hinge region, a second constant region (CH₂), and a third constant region (CH₃). In IgE and IgM antibodies, the heavy chain includes an additional constant region (CH₄).

Antibodies suitable for use with the present disclosure can include, for example, monoclonal antibodies, polyclonal antibodies, multispecific antibodies, human antibodies, humanized antibodies, camelid antibodies, chimeric antibodies, single-chain Fvs (scFv), disulfide-linked Fvs (sdFv), and anti-idiotypic (anti-Id) antibodies, and antigen-binding fragments of any of the above. In some embodiments, the antibody or antigen-binding fragment thereof is humanized. In some embodiments, the antibody or antigen-binding fragment thereof is chimeric. Antibodies can be of any type (e.g., IgG, IgE, IgM, IgD, IgA and IgY), class (e.g., IgG1, IgG2, IgG3, IgG4, IgA1 and IgA2) or subclass.

The term “antigen binding fragment” of an antibody, as used herein, refers to one or more fragments of an antibody that retain the ability to specifically bind to an antigen. Examples of binding fragments encompassed within the term “antigen binding fragment” of an antibody include a Fab fragment, a F(ab′)₂ fragment, a Fd fragment, a Fv fragment, a scFv fragment, a dAb fragment (Ward et al., (1989) Nature 341:544-546), and an isolated complementarity determining region (CDR). In some embodiments, an “antigen binding fragment” comprises a heavy chain variable region and a light chain variable region. These antibody fragments can be obtained using conventional techniques known to those with skill in the art, and the fragments can be screened for utility in the same manner as are intact antibodies.

Antibodies or antigen-binding fragments described herein can be produced by any method known in the art for the synthesis of antibodies (See. e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324: WO 98/46645). Chimeric antibodies can be produced using the methods described in, e.g., Morrison, 1985, Science 229:1202, and humanized antibodies by methods described in, e.g., U.S. Pat. No. 6,180,370.

Additional antibodies described herein are bispecific antibodies and multivalent antibodies, as described in, e.g., Segal et al., J. Immunol. Methods 248:1-6 (2001); and Tutt et al., J. Immunol. 147:60 (1991), or any of the molecules described herein.

“Avimer” relates to a multimeric binding protein or peptide engineered using, for example, in vitro exon shuffling and phage display. Multiple binding domains are linked, resulting in greater affinity and specificity compared to single epitope immunoglobin domains.

“Nanobodies” are antibody fragments consisting of a single monomeric variable antibody domain. Nanobodies may also be referred to as single-domain antibodies. Like antibodies, nanobodies are capable of binding selectively to a specific antigen. Nanobodies may be heavy-chain variable domains or light chain domains. Nanobodies may occur naturally or be the product of biological engineering. Nanobodies may be biologically engineered by site-directed mutagenesis or mutagenic screening (e.g., phage display, yeast display, bacterial display, mRNA display, ribosome display). “Affibodies” are polypeptides or proteins engineered to bind to a specific antigen. As such, affibodies may be considered to mimic certain functions of antibodies.

Affibodies may be engineered variants of the B-domain in the immunoglobulin-binding region of staphylococcal protein A. Affibodies may be engineered variants of the Z-domain, a B-domain that has lower affinity for the Fab region. Affibodies may be biologically engineered by site-directed mutagenesis or mutagenic screening (e.g., phage display, yeast display, bacterial display, mRNA display, ribosome display).

Affibody molecules showing specific binding to a variety of different proteins (e.g. insulin, fibrinogen, transferrin, tumor necrosis factor-α, IL-8, gp120, CD28, human serum albumin, IgA, IgE, IgM, HER2 and EGFR) have been generated, demonstrating affinities (Kd) in the μM to pM range. “Diabodies” are antibody fragments with two antigen-binding sites that may be bivalent or bispecific. See for example Hudson et al., (2003). Single-chain antibodies are antibody fragments comprising all or a portion of the heavy chain variable domain or all, or a portion of the light chain variable domain of an antibody. Antibody fragments can be made by various techniques including but not limited to proteolytic digestion of an intact antibody as well as production by recombinant hosts (e.g., E. coli or phage) as described herein.

In certain embodiments, the antibody or antigen-binding fragment thereof is a multispecific, e.g. bispecific. Multispecific antibodies (or antigen-binding fragments thereof) include monoclonal antibodies (or antigen-binding fragments thereof) that have binding specificities for at least two different sites.

The antibodies or antigen-binding fragments thereof that specifically bind to EGFRVIII may be an EGFRVIII antibody antigen-binding fragment thereof described in WO2020191485A1, which is incorporated by reference in its entirety.

In some embodiments, provided herein are EGFRvIII antibodies or antigen-binding fragments thereof selected from the group consisting of:
an antibody or antigen-binding fragment thereof comprising CDRL1 (SEQ ID NO: 38), CDRL2 (SEQ ID NO:39), CDRL3 (SEQ ID NO:40), CDRH1 (SEQ ID NO:43), CDRH2 (SEQ ID NO:44) and CDRH3 (SEQ ID NO:45) of the 4E11 antibody;
an antibody or antigen-binding fragment thereof comprising CDRL1 (SEQ ID NO: 8), CDRL2 (SEQ ID NO:9), CDR L³ (SEQ ID NO:10), CDRH1 (SEQ ID NO:13), CDRH2 (SEQ ID NO:14) and CDRH3 (SEQ ID NO:15) of the 5G6 antibody;
an antibody or antigen-binding fragment thereof comprising CDRL1 (SEQ ID NO: 18), CDRL2 (SEQ ID NO:19), CDRL3 (SEQ ID NO:20), CDRH1 (SEQ ID NO:23), CDRH2 (SEQ ID NO:24) and CDRH3 (SEQ ID NO:25) of the 1A8 antibody;
an antibody or antigen-binding fragment thereof comprising CDRL1 (SEQ ID NO: 28), CDRL2 (SEQ ID NO:29), CDRL3 (SEQ ID NO:30), CDRH1 (SEQ ID NO:33), CDRH2 (SEQ ID NO: 34) and CDRH3 (SEQ ID NO:35) of the 4B3 antibody;
an antibody or antigen-binding fragment thereof comprising CDRL1 (SEQ ID NO: 48), CDRL2 (SEQ ID NO:49), CDRL3 (SEQ ID NO:50), CDRH1 (SEQ ID NO:53), CDRH2 (SEQ ID NO:54) and CDRH3 (SEQ ID NO:55) of the 5D8 antibody;
an antibody or antigen-binding fragment thereof comprising CDRL1 (SEQ ID NO: 58), CDRL2 (SEQ ID NO:59), CDRL3 (SEQ ID NO:60), CDRH1 (SEQ ID NO:63), CDRH2 (SEQ ID NO:64), CDRH3 (SEQ ID NO:65) of the 9C₉ antibody; and
an antibody or antigen-binding fragment thereof comprising CDRL1 (SEQ ID NO: 68 or 73), CDRL2 (SEQ ID NO:69 or 74), CDRL3 (SEQ ID NO:70 or 75), CDRH1 (SEQ ID NO:78), CDRH2 (SEQ ID NO:79) and CDRH3 (SEQ ID NO:80) of the F266-11B1 (referred to herein as 11B1), F266-11C₈ (referred to herein as 11C₈), F266-11H5 (referred to herein as 11H5) and/or F266-11H3 (referred to herein as 11H3) antibodies.

In some embodiments, the antibody or antigen-binding fragment thereof comprise the CDRs of the 4E11 antibody.

In some embodiments, the antibody or antigen-binding fragment thereof comprise the CDRs of the 5G6 antibody,

In some embodiments, the antibody or antigen-binding fragment thereof comprise the CDRs of the 1A8 antibody.

In some embodiments, the antibody or antigen-binding fragment thereof comprise the CDRs of the 4B3 antibody.

In some embodiments, the antibody or antigen-binding fragment thereof comprise the CDRs of the 5D8 antibody.

In some embodiments, the antibody or antigen-binding fragment thereof comprise the CDRs of the 9C₉ antibody.

In some embodiments, the antibody or antigen-binding fragment thereof comprise the CDRs of the 11B1 or of the 11C₈ antibody.

In some embodiments, the present disclosure provides EGFRVIII antibodies or antigen-binding fragments thereof selected from the group consisting of:
an antibody or antigen-binding fragment thereof comprising CDR sequences consisting of CDRL1 (SEQ ID NO:8), CDRL2 (SEQ ID NO:9), CDRL3 (SEQ ID NO: 10), CDRH1 (SEQ ID NO:13), CDRH2 (SEQ ID NO: 14), CDRH3 (SEQ ID NO:15);
an antibody or antigen-binding fragment thereof comprising CDR sequences consisting of CDRL1 (SEQ ID NO:18), CDRL2 (SEQ ID NO:19), CDRL3 (SEQ ID NO:20), CDRH1 (SEQ ID NO:23), CDRH2 (SEQ ID NO:24), CDRH3 (SEQ ID NO:25);
an antibody or antigen-binding fragment thereof comprising CDR sequences consisting of CDRL1 (SEQ ID NO:28), CDRL2 (SEQ ID NO:29), CDRL3 (SEQ ID NO:30), CDRH1 (SEQ ID NO:33), CDRH2 (SEQ ID NO:34), CDRH3 (SEQ ID NO:35);
an antibody or antigen-binding fragment thereof comprising CDR sequences consisting of CDRL1 (SEQ ID NO:38), CDRL2 (SEQ ID NO:39), CDRL3 (SEQ ID NO:40), CDRH1 (SEQ ID NO:43), CDRH2 (SEQ ID NO:44), CDRH3 (SEQ ID NO:45);
an antibody or antigen-binding fragment thereof comprising CDR sequences consisting of CDRL1 (SEQ ID NO:48). CDRL2 (SEQ ID NO:49). CDRL3 (SEQ ID NO:50). CDRH1 (SEQ ID NO:53). CDRH2 (SEQ ID NO:54). CDRH3 (SEQ ID NO:55);
an antibody or antigen-binding fragment thereof comprising CDR sequences consisting of CDRL1 (SEQ ID NO:58). CDRL2 (SEQ ID NO:59). CDRL3 (SEQ ID NO:60). CDRH1 (SEQ ID NO:63). CDRH2 (SEQ ID NO:64). CDRH3 (SEQ ID NO:65);
an antibody or fragment thereof comprising CDR sequences consisting of CDRL1 (SEQ ID NO:68). CDRL2 (SEQ ID NO:69). CDRL3 (SEQ ID NO:70). CDRH1 (SEQ ID NO: 78). CDRH2 (SEQ ID NO:79). CDRH3 (SEQ ID NO:80); and
an antibody or antigen-binding fragment thereof comprising CDR sequences consisting of CDRL1 (SEQ ID NO:73). CDRL2 (SEQ ID NO:74). CDRL3 (SEQ ID NO:75). CDRH1 (SEQ ID NO:78). CDRH2 (SEQ ID NO:79). CDRH3 (SEQ ID NO:80).

In some embodiments, the present disclosure provides an antibody or an antigen-binding fragment thereof, which specifically binds to EGFRVIII and which may comprise for example:
a light chain variable region which may comprise a CDRL1 having the amino acid sequence set forth in SEQ ID NO:8, a CDRL2 having the amino acid sequence set forth in SEQ ID NO:9 and a CDRL3 having the amino acid sequence set forth in SEQ ID NO:10 and a heavy chain variable region which may comprise a CDRH1 having the amino acid sequence set forth in SEQ ID NO:13, a CDRH2 having the amino acid sequence set forth in SEQ ID NO:14 and a CDRH3 having the amino acid sequence set forth in SEQ ID NO:15;
a light chain variable region which may comprise a CDRL1 having the amino acid sequence set forth in SEQ ID NO: 18, a CDRL2 having the amino acid sequence set forth in SEQ ID NO: 19 and a CDRL3 having the amino acid sequence set forth in SEQ ID NO:20 and a heavy chain variable region which may comprise a CDRH1 having the amino acid sequence set forth in SEQ ID NO:23, a CDRH2 having the amino acid sequence set forth in SEQ ID NO:24 and a CDRH3 having the amino acid sequence set forth in SEQ ID NO:25;
a light chain variable region which may comprise a CDRL1 having the amino acid sequence set forth in SEQ ID NO:28, a CDRL2 having the amino acid sequence set forth in SEQ ID NO:29 and a CDRL3 having the amino acid sequence set forth in SEQ ID NO:30 and a heavy chain variable region which may comprise a CDRH1 having the amino acid sequence set forth in SEQ ID NO:33, a CDRH2 having the amino acid sequence set forth in SEQ ID NO:34 and a CDRH3 having the amino acid sequence set forth in SEQ ID NO:35;
a light chain variable region which may comprise a CDRL1 having the amino acid sequence set forth in SEQ ID NO:38, a CDRL2 having the amino acid sequence set forth in SEQ ID NO:39 and a CDRL3 having the amino acid sequence set forth in SEQ ID NO:40 and a heavy chain variable region which may comprise a CDRH1 having the amino acid sequence set forth in SEQ ID NO:43, a CDRH2 having the amino acid sequence set forth in SEQ ID NO:44 and a CDRH3 having the amino acid sequence set forth in SEQ ID NO:45;
a light chain variable region which may comprise a CDRL1 having the amino acid sequence set forth in SEQ ID NO:48, a CDRL2 having the amino acid sequence set forth in SEQ ID NO:49 and a CDRL3 having the amino acid sequence set forth in SEQ ID NO:50 and a heavy chain variable region which may comprise a CDRH1 having the amino acid sequence set forth in SEQ ID NO:53, a CDRH2 having the amino acid sequence set forth in SEQ ID NO:54 and a CDRH3 having the amino acid sequence set forth in SEQ ID NO:55;
a light chain variable region which may comprise a CDRL1 having the amino acid sequence set forth in SEQ ID NO:58, a CDRL2 having the amino acid sequence set forth in SEQ ID NO:59 and a CDRL3 having the amino acid sequence set forth in SEQ ID NO:60 and a heavy chain variable region which may comprise a CDRH1 having the amino acid sequence set forth in SEQ ID NO:63, a CDRH2 having the amino acid sequence set forth in SEQ ID NO:64 and a CDRH3 having the amino acid sequence set forth in SEQ ID NO:65;
a light chain variable region which may comprise a CDRL1 having the amino acid sequence set forth in SEQ ID NO:68, a CDRL2 having the amino acid sequence set forth in SEQ ID NO:69 and a CDRL3 having the amino acid sequence set forth in SEQ ID NO:70 and a heavy chain variable region which may comprise a CDRH1 having the amino acid sequence set forth in SEQ ID NO:78, a CDRH2 having the amino acid sequence set forth in SEQ ID NO:79 and a CDRH3 having the amino acid sequence set forth in SEQ ID NO:80; or
a light chain variable region which may comprise a CDRL1 having the amino acid sequence set forth in SEQ ID NO:73, a CDRL2 having the amino acid sequence set forth in SEQ ID NO:74 and a CDRL3 having the amino acid sequence set forth in SEQ ID NO:75 and a heavy chain variable region which may comprise a CDRH1 having the amino acid sequence set forth in SEQ ID NO:78, a CDRH2 having the amino acid sequence set forth in SEQ ID NO:79 and a CDRH3 having the amino acid sequence set forth in SEQ ID NO:80.

In some embodiments, the present disclosure provides an antibody or an antigen-binding fragment thereof, which specifically binds to EGFRVIII and which may comprise:
a light chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 118 or substantially identical to SEQ ID NO: 118 and/or a heavy chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 116 or substantially identical to SEQ ID NO:116;
a light chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 115 or substantially identical to SEQ ID NO: 115 and a heavy chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 116 or substantially identical to SEQ ID NO:116; or
a light chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 118 or substantially identical to SEQ ID NO: 118 and a heavy chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO:62 or substantially identical to SEQ ID NO:62.

In certain embodiments, antibodies or antigen-binding fragments thereof having light chain at least 80%, at least 85%, at least 90%, at least 92%, at least 94%, at least 95%, at least 96%, at least 97%, at least 98%, or at least 99% identical or substantially identical to the amino acid sequence set forth in SEQ ID NO: 115 or SEQ ID NO: 118 may have CDRs identical to those of SEQ ID NO: 115 or SEQ ID NO: 118 respectively.

In certain embodiments, antibodies or antigen-binding fragments thereof having heavy chain at least 80%, at least 85%, at least 90%, at least 92%, at least 94%, at least 95%, at least 96%, at least 97%, at least 98%, or at least 99% identical or substantially identical to the amino acid sequence set forth in SEQ ID NO: 62 or SEQ ID NO: 116 may have CDRs identical to those of SEQ ID NO: 62 or SEQ ID NO: 116 respectively.

In some embodiments, the present disclosure provides an antibody or an antigen-binding fragment thereof, which specifically binds to EGFRVIII and which may comprise:
a light chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 7 or substantially identical to SEQ ID NO:7 and a heavy chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 12 or substantially identical to SEQ ID NO: 12;
a light chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 17 or substantially identical to SEQ ID NO: 17 and a heavy chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO:22 or substantially identical to SEQ ID NO: 22;
a light chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 27 or substantially identical to SEQ ID NO:27 and a heavy chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO:32 or substantially identical to SEQ ID NO: 32;
a light chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 37 or substantially identical to SEQ ID NO:37 and a heavy chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO:42 or substantially identical to SEQ ID NO: 42;
a light chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 47 or substantially identical to SEQ ID NO:47 and a heavy chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO:52 or substantially identical to SEQ ID NO: 52;
a light chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 57 or substantially identical to SEQ ID NO:57 and a heavy chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO:62 or substantially identical to SEQ ID NO: 62;
a light chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 67 or substantially identical to SEQ ID NO:67 and a heavy chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO:77 or substantially identical to SEQ ID NO: 77, at least 80%, at least 85%, at least 90%, at least 92%, at least 94%, at least 95%, at least 96%, at least 97%, at least 98%, or at least 99% identical to the amino acid set forth in SEQ ID NO:92 or substantially identical to SEQ ID NO:92 or at least 80%, at least 85%, at least 90%, at least 92%, at least 94%, at least 95%, at least 96%, at least 97%, at least 98%, or at least 99% identical to the amino acid sequence set forth in SEQ ID NO: 102 or substantially identical to SEQ ID NO:102; or
a light chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 72 or substantially identical to SEQ ID NO: 72 and a heavy chain variable region which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO:77 or substantially identical to SEQ ID NO: 77 or at least 80%, at least 85%, at least 90%, at least 92%, at least 94%, at least 95%, at least 96%, at least 97%, at least 98%, or at least 99% identical to the amino acid set forth in SEQ ID NO:92 or substantially identical to SEQ ID NO:92.

In various embodiments, the light chain variable regions, light chains, heavy chain variable regions or heavy chains which may comprise an amino acid sequence at least 80%, at least 85%, at least 90%, at least 92%, at least 94%, at least 95%, at least 96%, at least 97%, at least 98%, or at least 99% identical to that of given antibody may have CDRs that are identical to that antibody. In some embodiments, the VL and VH sequences of the antibodies and antigen-binding fragments provided in the present disclosure may comprise a sequence substantially identical to the VL and VH sequences provided herein, or may comprise a sequence having at least 80%, at least 85%, at least 90%, at least 92%, at least 94%, at least 95%, at least 96%, at least 97%, at least 98%, or at least 99% sequence identity, wherein sequence variation is preferably outside the CDRs of the VL and VH sequences provided.

In further embodiments, the present disclosure provides an antibody or an antigen-binding fragment thereof, which specifically binds to EGFRVIII and which may comprise:
a light chain which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 108 or substantially identical to SEQ ID NO: 108 and a heavy chain which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO:107 or substantially identical to SEQ ID NO:107; or
a light chain which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO:110 or substantially identical to SEQ ID NO: 110 and a heavy chain which may comprise an amino acid sequence at least 80%, at least 85%, at least 90%, at least 92%, at least 94%, at least 95%, at least 96%, at least 97%, at least 98%, or at least 99% identical to the amino acid sequence set forth in SEQ ID NO: 109 or substantially identical to SEQ ID NO:109.

In some embodiments, the present disclosure further provides anti-EGFRvIII antibodies or antigen-binding fragments thereof, which may comprise:
a CDRL1 having the amino acid sequence set forth in SEQ ID NO:38, a CDRL2 having the amino acid sequence set forth in SEQ ID NO:39 and a CDRL3 having the amino acid sequence set forth in SEQ ID NO:40, a CDRH1 having the amino acid sequence set forth in SEQ ID NO:43, a CDRH2 having the amino acid sequence set forth in SEQ ID NO: 44 and a CDRH3 having the amino acid sequence set forth in SEQ ID NO:45;
a light chain variable region which may comprise an amino acid sequence at least 80% identical, at least 85% identical, at least 90% identical, at least 92% identical, at least 94% identical, at least 95% identical, at least 96% identical, at least 97% identical, at least 98% identical, at least 99% identical or identical to the amino acid sequence set forth in SEQ ID NO: 37 and a heavy chain variable region which may comprise an amino acid sequence at least 80% identical, at least 85% identical, at least 90% identical, at least 92% identical, at least 94% identical, at least 95% identical, at least 96% identical, at least 97% identical, at least 98% identical, at least 99% identical or identical to the amino acid sequence set forth in SEQ ID NO:42; or
a light chain which may comprise an amino acid sequence at least 80% identical, at least 85% identical, at least 90% identical, at least 92% identical, at least 94% identical, at least 95% identical, at least 96% identical, at least 97% identical, at least 98% identical, at least 99% identical or identical to the amino acid sequence set forth in SEQ ID NO: 108 and a heavy chain which may comprise an amino acid sequence at least 80% identical, at least 85% identical, at least 90% identical, at least 92% identical, at least 94% identical, at least 95% identical, at least 96% identical, at least 97% identical, at least 98% identical, at least 99% identical or identical to the amino acid sequence set forth in SEQ ID NO:107.

In accordance with the present disclosure, the antibody or antigen-binding fragment thereof set forth above may have CDRs identical or substantially identical to those set forth in SEQ ID NOs: 38, 39, 40, 43, 44 and 45.

In some embodiments, the present disclosure also provides EGFRvIII antibodies or antigen-binding fragments thereof, comprising light chain sequences that comprise the signal sequence MVLQTQVFISLLLWISGAYG (SEQ ID NO: 113) at the N-terminus, and heavy chain sequences that comprise the signal sequence MDWTWRILFLVAAATGTHA (SEQ ID NO:114) at the N-terminus. In certain embodiments, each of the light chain sequences set forth in SEQ ID NOs: 180, 181, and 182 comprises the signal sequence MVLQTQVFISLLLWISGAYG (SEQ ID NO:113) at the N-terminus. In certain embodiments, each of the heavy chain sequences set forth in SEQ ID NOs: 183, 184, and 185 comprises the signal sequence MDWTWRILFLVAAATGTHA (SEQ ID NO: 114) at the N-terminus.

In accordance with the present disclosure, the antibody or antigen-binding fragment thereof may have, for example, an affinity to EGFRVIII of less than 100 nM, such as an affinity to EGFRvIII of 50 nM or less, 20 nM or less, 10 nM or less, or 5 nM or less.

Exemplary embodiments of the present disclosure include antibodies or antigen-binding fragments thereof which may comprise a human IgG constant region. Antibodies or antigen-binding fragments of the present disclosure may comprise, for example and without limitation, a human IgG1 constant region or a human IgG2 constant region or a human IgG4 constant region, or a mutational variant thereof.

In an exemplary embodiment, the antigen-binding agents disclosed herein may comprise humanized framework regions.

In accordance with the present disclosure, the antibody or antigen-binding fragment thereof may be monoclonal antibody, a polyclonal antibody, a humanized antibody, a chimeric antibody, a human antibody, a single chain antibody, or a multispecific antibody (e.g., a bispecific antibody).

Bispecific antibodies or antigen-binding fragments thereof of the present disclosure includes those that may comprise a first targeting moiety that specifically binds to a first human EGFRvIII epitope and a second targeting moiety that specifically binds to a second (non-overlapping) human EGFRvIII epitope (e.g., a biparatopic antibody).

Additional embodiments of bispecific antibodies or antigen-binding fragments thereof of the present disclosure includes those that may comprise a first targeting moiety that specifically binds to a first human EGFRvIII epitope and a second targeting moiety that specifically binds to another antigen.

The bispecific antibody or antigen-binding fragment thereof of the present disclosure include bispecific immune cell engagers such as those comprising a first targeting moiety that specifically binds to human EGFRVIII and a second targeting moiety that specifically binds to CD3.

In accordance with the present disclosure, the antigen-binding fragment of an EGFRVIII antibody may comprise, for example, a scFv, a Fab, a Fab′ or a (Fab′)₂.

### Polypeptides

Polypeptides include, for example, any of a variety of hematologic agents (including, for instance, erythropoietin, blood-clotting factors, etc.), interferons, colony stimulating factors, antibodies, enzymes, and hormones. The identity of a particular polypeptide is not intended to limit the present disclosure, and any polypeptide of interest can be a polypeptide in the present methods.

A reference polypeptide described herein can include a target-binding domain that is capable of binding to a target of interest (e.g., is capable of binding to an antigen, e.g., EGFRvIII). For example, a polypeptide, such as an antibody, can bind to a transmembrane polypeptide (e.g., receptor) or ligand (e.g., a growth factor).

### Modified Polypeptides

Polypeptides suitable for use with compositions and methods of the present disclosure may have a modified amino acid sequence. Modified polypeptides may be substantially identical to the corresponding reference polypeptide (e.g., the amino acid sequence of the modified polypeptide may have at least 50%, 60%, 70%, 75%, 80%, 85%, 90%, 95%, 96%, 97%, 98%, 99%, or 100% identity to the amino acid sequence of the reference polypeptide). In certain embodiments, the modification does not destroy significantly a desired biological activity (e.g., binding to EGFRVIII). The modification may reduce (e.g., by at least 5%, 10%, 20%, 25%, 35%, 50%, 60%, 70%, 75%, 80%, 90%, or 95%), may have no effect, or may increase (e.g., by at least 5%, 10%, 25%, 50%, 100%, 200%, 500%, or 1000%) the biological activity of the original polypeptide. The modified polypeptide may have or may optimize a characteristic of a polypeptide, such as in vivo stability, bioavailability, toxicity, immunological activity, immunological identity, and conjugation properties.

Modifications include those by natural processes, such as post-translational processing, or by chemical modification techniques known in the art. Modifications may occur anywhere in a polypeptide including the polypeptide backbone, the amino acid side chains and the amino- or carboxy-terminus. The same type of modification may be present in the same or varying degrees at several sites in a given polypeptide, and a polypeptide may contain more than one type of modification. Polypeptides may be branched as a result of ubiquitination, and they may be cyclic, with or without branching. Cyclic, branched, and branched cyclic polypeptides may result from post-translational natural processes or may be made synthetically. Other modifications include pegylation, acetylation, acylation, addition of acetomidomethyl (Acm) group, ADP-ribosylation, alkylation, amidation, biotinylation, carbamoylation, carboxyethylation, esterification, covalent attachment to flavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristovlation, oxidation, proteolytic processing, phosphorylation, prenylation, racemization, selenoylation, sulfation, transfer-RNA mediated addition of amino acids to proteins such as arginylation and ubiquitination.

A modified polypeptide can also include an amino acid insertion, deletion, or substitution, either conservative or non-conservative (e.g., D-amino acids, desamino acids) in the polypeptide sequence (e.g., where such changes do not substantially alter the biological activity of the polypeptide). In particular, the addition of one or more cysteine residues to the amino or carboxy-terminus of a polypeptide herein can facilitate conjugation of these polypeptides by, e.g., disulfide bonding. For example, a polypeptide can be modified to include a single cysteine residue at the amino-terminus or a single cysteine residue at the carboxy-terminus. Amino acid substitutions can be conservative (i.e., wherein a residue is replaced by another of the same general type or group) or non-conservative (i.e., wherein a residue is replaced by an amino acid of another type). In addition, a naturally occurring amino acid can be substituted for a non-naturally occurring amino acid (i.e., non-naturally occurring conservative amino acid substitution or a non-naturally occurring non-conservative amino acid substitution).

Polypeptides made synthetically can include substitutions of amino acids not naturally encoded by DNA (e.g., non-naturally occurring or unnatural amino acid). Examples of non-naturally occurring amino acids include D-amino acids, N-protected amino acids, an amino acid having an acetylaminomethyl group attached to a sulfur atom of a cysteine, a pegylated amino acid, the omega amino acids of the formula NH₂ (CH₂) COOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs may be generated by substitutional mutagenesis and retain the biological activity of the original polypeptide. Examples of substitutions identified as “conservative substitutions” are shown in Table 1. If such substitutions result in a change not desired, then other type of substitutions, denominated “exemplary substitutions” in Table 1, or as further described herein in reference to amino acid classes, are introduced and the products screened.

TABLE 1
Amino acid substitutions

| Original residue | Exemplary substitution | Conservative substitution |
|---|---|---|
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

Substantial modifications in function or immunological identity are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, and/or (c) the bulk of the side chain.

### Chelating Moiety or Metal Complex Thereof

### Chelating Moieties

Examples of suitable chelating moieties include, but are not limited to, DOTA (1,4,7,10-tetraazacyclododecane-1,4,7,10-tetraacetic acid), DOTMA (1R,4R,7R,10R)-α,α′, α″,α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), DO3AM-acetic acid (2-(4,7,10-tris(2-amino-2-oxoethyl)-1,4,7,10-tetraazacyclododecan-1-yl) acetic acid), DOTA-GA anhydride (2,2′,2″-(10)-(2,6-dioxotetrahydro-2H-pyran-3-yl)-1,4,7,10-tetraazacyclododecane-1,4,7-triyl)triacetic acid, DOTP (1,4,7,10-tetraazacyclododecane-1,4,7,10-tetra(methylene phosphonic acid)), DOTMP (1,4,6,10-tetraazacyclodecane-1,4,7,10-tetramethylene phosphonic acid, DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacy clotetradecane-1,4,8,11-tetrapropionic acid), TETA (1,4,8,11-tetraazacyclotetradecane-1,4,8,11-tetra acetic acid), HEHA (1,4,7,10,13,16-hexaazacyclohexadecane-1,4,7,10,13,16-hexaacetic acid), PEPA (1,4,7,10,13-pentaazacyclopentadecane-N,N′,N″,N′″, N″″-pentaacetic acid), H₄octapa (N,N′-bis(6-carboxy-2-pyridylmethyl)-ethylenediamine-N,N′-diacetic acid), H₂dedpa (1,2-[[6-(carboxy)-pyridin-2-v1]-methylamino]ethane), Hophospa (N,N′-(methylenephosphonate)-N,N′-[6-(methoxycarbonyl)pyridin-2-v1]-methyl-1,2-diaminoethane), TTHA (triethylenetetramine-N,N,N′,N″,N′″, N′″-hexaacetic acid), DO2P (tetraazacyclododecane dimethanephosphonic acid), HP-DO3A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), octadentate-HOPO (octadentate hydroxypyridinones), or porphyrins.

Preferably, the chelating moiety is selected from DOTA (1,4,7,10-tetraazacyclododecane-1,4,7,10-tetraacetic acid), DOTMA (1R,4R,7R,10R)-α,α′,α″,α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DO3AM-acetic acid (2-(4,7,10-tris(2-amino-2-oxoethyl)-1,4,7,10-tetraazacyclododecan-1-yl) acetic acid), DOTP (1,4,7,10-tetraazacyclododecane-1,4,7,10-tetra(methylene phosphonic acid)), DOTA-4AMP (1,4,7,10-tetraazacyclododecane-1,4,7,10-tetrakis(acetamido-methylenephosphonic acid), NOTA (1,4,7-triazacyclononane-1,4,7-triacetic acid), and HP-DO3A (10-(2-hydroxypropyl)-1,4,7-tetraazacyclododecane-1,4,7-triacetic acid).

In some embodiments, the chelating moiety is DOTA.

In some embodiments, compounds comprise a metal complex of a chelating moiety. For example, chelating groups may be used in metal chelate combinations with metals, such as manganese, iron, and gadolinium and isotopes (e.g., isotopes in the general energy range of 60 to 10,000 keV), such as any of the radioisotopes and radionuclides discussed herein.

In some embodiments, chelating moieties are useful as detection agents, and compounds comprising such detectable chelating moieties can therefore be used as diagnostic or theranostic agents.

In some embodiments, variable A of Formula I is a macrocyclic chelating moiety comprising one or more heteroaryl groups (e.g., six-membered nitrogen-containing heteroaryl). Examples of such macrocyclic chelating moiety include, but are not limited to:

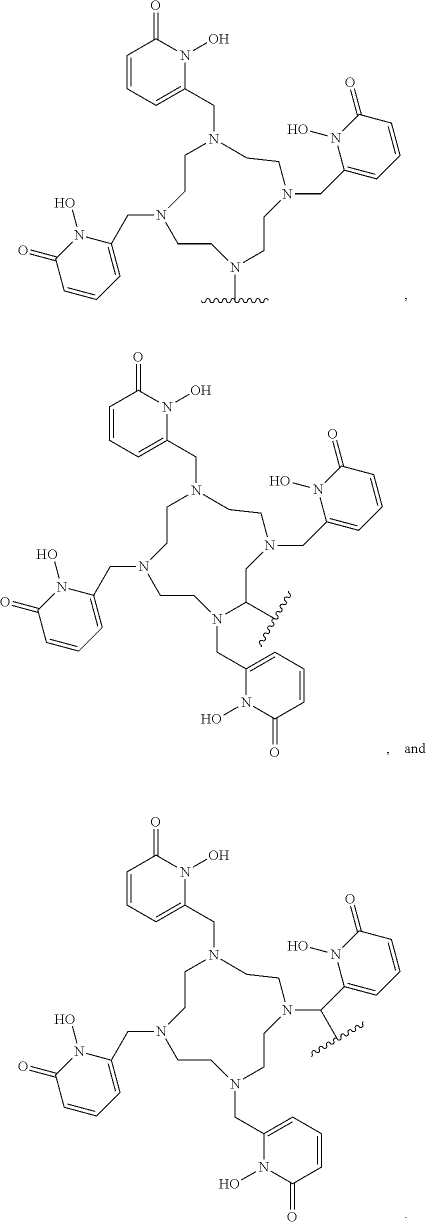 [SMILES: *N1CCN(Cc2cccc(=O)n2O)CCN(Cc2cccc(=O)n2O)CCN(Cc2cccc(=O)n2O)CC1 ; *C1CN(Cc2cccc(=O)n2O)CCN(Cc2cccc(=O)n2O)CCN(Cc2cccc(=O)n2O)CCN1Cc1cccc(=O)n1O ; *C(c1cccc(=O)n1O)N1CCN(Cc2cccc(=O)n2O)CCN(Cc2cccc(=O)n2O)CCN(Cc2cccc(=O)n2O)CC1]

### Radioisotopes and Radionuclides

In some embodiments, the metal complex comprises a radionuclide. Examples of suitable radioisotopes and radionuclides include, but are not limited to, ³H, ¹⁴C, ¹⁵N, ¹⁸F, ³⁵S, ⁴³Sc, ++Sc, ⁴⁷Sc, ⁵⁵Co, ⁶⁰Cu, ⁶¹Cu, ⁶²Cu, ⁶⁴Cu, ⁶⁶Ga, ⁶⁷Ga, ⁶⁷Cu, ⁶⁸Ga, ⁷⁵Br, ⁷⁶Br, ⁷⁷Br, ⁸²Rb, ⁸⁹Zr, ⁸⁶Y, ⁸⁷Y, ⁹⁰Y, ⁹⁷Ru, ⁹⁹Tc, ^{99m}Tc, ¹⁰⁵Rh, ¹⁰⁹Pd, ¹¹¹In, ¹²³I, ¹²⁴I, ¹²⁵I, ¹³¹I, ¹³³La, ¹³⁴Ce, ¹⁴⁹Pm, ¹⁴⁹Tb, ¹⁵³Sm, ¹⁶⁶Ho, ¹⁷⁷Lu, ^{117m}Sn, ¹⁸⁶Re, ¹⁸⁸Re, ¹⁹⁸Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ²²⁷Th, and ²²⁹Th.

In some embodiments, the metal complex comprises a radionuclide selected from ⁴³Sc, +Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁸⁹Zr, ⁹⁰Y, ⁹⁷Ru, ⁹⁹Tc, ^{99m}Tc, ¹⁰⁵Rh, ¹⁰⁹Pd, ¹¹¹In, ^{117m}Sn, ¹³³La, ¹³⁴Ce, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰¹Tl, ²⁰³pb, ²¹¹At, ²¹²Pb, ²¹²Bi, ²¹³Bi, ²²³Ra, ²²⁵Ac, ²²⁷Th, and ²²⁹Th. In certain embodiments, the metal complex comprises a radionuclide selected from ⁶⁸Ga, ⁸⁹Zr, ⁹⁰Y, ¹¹¹In, ¹⁷⁷Lu, and ²²⁵Ac. In certain embodiments, the metal complex comprises a radionuclide being ¹⁷⁷Lu or ²²⁵Ac.

In some embodiments, the radionuclide is an alpha emitter, e.g., Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), or Terbium-149 (¹⁴⁹Tb), or a progeny thereof. In some embodiments, the alpha-emitter is Actinium-225 (²²⁵Ac), or a progeny thereof.

### Linker

The compounds of this invention generally comprise the structure of Formula I below:

A-L¹-(L²)ₙ-B Formula I

wherein each of the variables is defined in the SUMMARY section above.

Each of the compounds of Formula I comprises a linker moiety as -L¹-(L²)ₙ-, wherein:

L¹ is a bond, C—O, C═S, optionally substituted C₁-C₆ alkyl, optionally substituted C₁-C₆ heteroalkyl, optionally substituted aryl, or optionally substituted heteroaryl; n is an integer between 1 and 5 (inclusive); and each L², independently, has the structure:

—X¹-L³-Z¹— Formula II

wherein:
X¹ is —C(O)NR¹—*, —NR¹C(O)—*, —C(S)NR¹—*, —NR¹C(S)—*, —OC(O)NR¹—*—NR¹C(O)O—*, —NR¹C(O)NR¹—*, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—*, or —NR¹—*; wherein “*” indicates the attachment point to L³, and R¹ is hydrogen, optionally substituted C₁-C₆ alkyl, optionally substituted C₁-C₆ heteroalkyl, or optionally substituted aryl or heteroaryl;
L³ is optionally substituted C₁-C₅₀ alkyl or optionally substituted C₁-C₅₀ heteroalkyl (e.g., (CH₂CH₂O) 2-20); and
Z¹ is —CH₂-#, —C(O)-#, —C(S)-#, —OC(O)-#, —C(O)O-#, —NR²C(O)-#, —C(O)NR²-#, or —NR²-#, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl.

In some embodiments, L¹ is optionally substituted C₁-C₆ alkyl or optionally substituted C₁-C₆ heteroalkyl. In certain embodiments, L¹ is substituted C₁-C₆ alkyl or substituted C₁-C₆ heteroalkyl, the substituent comprising a heteroaryl group (e.g., six-membered nitrogen-containing heteroaryl). In some embodiments, L¹ is C₁-C₆ alky. For example, L¹ is —CH₂CH₂—. In some embodiments, L¹ is a bond. In some embodiments, L¹ is

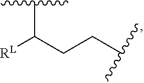

wherein R^{L} is hydrogen or —CO₂H.

In some embodiments, X¹ is —C(O)NR¹—*, —NR¹C(O)—*, or —NR¹_“*” indicating the attachment point to L³, and R¹ is hydrogen, optionally substituted C₁-C₆ alkyl, optionally substituted C₁-C₆ heteroalkyl, optionally substituted aryl, or optionally substituted heteroaryl. In some embodiments, X¹ is —C(O)NR¹_*, “*” indicating the attachment point to L³, and R¹ is hydrogen.

In some embodiments, L³ is optionally substituted C₁-C₅₀ alkyl (e.g., C₃-C₃₀ alkyl, C₃-C₂₅ alkyl, C₃-C₂₀ alkyl, C₃-C₁₅ alkyl, C₃-C₁₀ alkyl, C₅-C₃₀ alkyl, C₅-C₂₅ alkyl, C₅-C₂₀ alkyl, C₅-C₁₅ alkyl, and C₅-C₁₀ alkyl) or optionally substituted C₁-C₅₀ heteroalkyl (e.g., C₃-C₃₀ heteroalkyl, C₃-C₂₅ heteroalkyl, C₃-C₂₀ heteroalkyl, C₃-C₁₅ heteroalkyl, C₃-C₁₀ heteroalkyl, C₅-C₃₀ heteroalkyl, C₅-C₂₅ heteroalkyl, C₅-C₂₀ heteroalkyl, C₅-C₁₅ heteroalkyl, and C₅-C₁₀ heteroalkyl). An exemplary C₁-C₅₀ heteroalkyl is C₅-C₃₀ polyethylene glycol (e.g., C₅-C₂₅ polyethylene glycol, C₅-C₂₀ polyethylene glycol, C₅-C₁₅ polyethylene glycol). In certain embodiments, L³ is C₅-C₂₅ polyethylene glycol, C₅-C₂₀ polyethylene glycol, or C₅-C₁₅ polyethylene glycol.

In some embodiments, L³ is optionally substituted C₁-C₅₀ heteroalkyl (e.g., C₁-C₄₀ heteroalkyl, C₁-C₃₀ heteroalkyl, C₁-C₂₀ heteroalkyl, C₂-C₁₈ heteroalkyl, C₃-C₁₆ heteroalkyl, C₄-C₁₄ heteroalkyl, C₅-C₁₂ heteroalkyl, C₆-C₁₀ heteroalkyl, C₅-C₁₀ heteroalkyl, C₄ heteroalkyl, C₆ heteroalkyl, C₈ heteroalkyl, C₁₀ heteroalkyl, C₁₂ heteroalkyl, C₁₆ heteroalkyl, C₂₀ heteroalkyl, or C₂₄ heteroalkyl).

In some embodiments, L³ is optionally substituted C₁-C₅₀ heteroalkyl comprising a polyethylene glycol (PEG) moiety comprising 1-20 oxyethylene (—O—CH₂—CH₂—) units, e.g., 2 oxyethylene units (PEG2), 3 oxyethylene units (PEG3), 4 oxyethylene units (PEG4), 5 oxyethylene units (PEG5), 6 oxyethylene units (PEG6), 7 oxyethylene units (PEG7), 8 oxyethylene units (PEG8), 9 oxyethylene units (PEG9), 10 oxyethylene units (PEG10), 12 oxyethylene units (PEG12), 14 oxyethylene units (PEG14), 16 oxyethylene units (PEG16), or 18 oxyethylene units (PEG18).

In certain embodiments, L³ is optionally substituted C₁-50 heteroalkyl comprising a polyethylene glycol (PEG) moiety comprising 1-20 oxyethylene (—O—CH₂—CH₂—) units or portions thereof. For example, L³ comprises PEG3 as shown below:

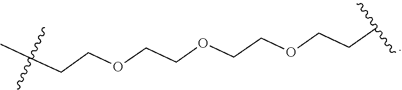 [SMILES: *CCOCCOCCOCC*]

In some embodiments, L³ is (CH₂CH₂O)ₘ(CH₂) w, and m and w are each independently an integer between 0 and 10 (inclusive), and at least one of m and w is not 0.

In some embodiments, L³ is substituted C₁-C₅₀ alkyl or substituted C₁-C₅₀ heteroalkyl, the substituent comprising a heteroaryl group (e.g., six-membered nitrogen-containing heteroaryl).

In some embodiments, Z¹ is CH₂, C═O, or NR¹; wherein R¹ is H, optionally substituted C₁-C₆ alkyl, optionally substituted C₁-C₆ heteroalkyl, optionally substituted aryl, or optionally substituted heteroaryl.

In certain embodiments, A-L¹-(L²), —B can be represented by the following structure:

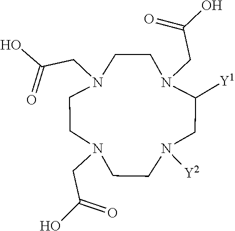

wherein Y¹ is —CH₂OCH₂ (L²)ₙ-B, C═O(L²)ₙ-B, or C═S(L²)ₙ-B and Y² is —CH₂CO₂H; or wherein Y¹ is H and Y² is L¹-(L²)ₙ-B.

### Cross-Linking Groups

In some embodiments, compounds (e.g., radioimmunoconjugates) are synthesized using bifunctional chelates that comprise a chelate, a linker, and a cross-linking group. Once the compound (e.g., radioimmunoconjugate) is formed, the cross-linking group may be absent from the compound (e.g., radioimmunoconjugate).

In some embodiments, compounds (e.g., radioimmunoconjugates) comprise a cross-linking group instead of or in addition to the targeting moiety (e.g., in some embodiments, B in Formula I comprises a cross-linking group).

A cross-linking group is a reactive group that is able to join two or more molecules by a covalent bond. Cross-linking groups may be used to attach the linker and chelating moiety to a therapeutic or targeting moiety. Cross-linking groups may also be used to attach the linker and chelating moiety to a target in vivo. In some embodiments, the cross-linking group is an amino-reactive, methionine reactive or thiol-reactive cross-linking group, or a comprises sortase recognition sequence. In some embodiments, the amino-reactive or thiol-reactive cross-linking group comprises an activated ester such as a hydroxysuccinimide ester, 2,3,5,6-tetrafluorophenol ester, 4-nitrophenol ester or an imidate, anhydride, thiol, disulfide, maleimide, azide, alkyne, strained alkyne, strained alkene, halogen, sulfonate, haloacetyl, amine, hydrazide, diazirine, phosphine, tetrazine, isothiocyanate, or oxaziridine. In some embodiments, the sortase recognition sequence may comprise of a terminal glycine-glycine-glycine (GGG) and/or LPTXG amino acid sequence, where X is any amino acid. A person having ordinary skill in the art will understand that the use of cross-linking groups is not limited to the specific constructs disclosed herein, but rather may include other known cross-linking groups.

### Pharmaceutical Compositions

In one aspect, the present disclosure provides pharmaceutical compositions comprising compounds disclosed herein. Such pharmaceutical compositions can be formulated for use in a variety of drug delivery systems. One or more physiologically acceptable excipients or carriers can also be included in a pharmaceutical composition for proper formulation. Non-limiting examples of suitable formulations compatible for use with the present disclosure include those described in Remington's Pharmaceutical Sciences, Mack Publishing Company, Philadelphia, PA, 17th ed., 1985. For a brief review of methods for drug delivery, See. e.g., Langer (Science. 249:1527-1533, 1990).

Pharmaceutical compositions may be formulated for any of a variety of routes of administration discussed herein (See. e.g., the “Administration and Dosage” subsection herein), Sustained release administration is contemplated, by such means as depot injections or erodible implants or components. Thus, the present disclosure provides pharmaceutical compositions that include agents disclosed herein (e.g., radioimmunoconjugates) dissolved or suspended in an acceptable carrier, preferably an aqueous carrier, e.g., water, buffered water, saline, or PBS, among others. In some embodiments, pharmaceutical compositions contain pharmaceutically acceptable auxiliary substances to approximate physiological conditions, such as pH adjusting and buffering agents, tonicity adjusting agents, wetting agents, or detergents, among others. In some embodiments, pharmaceutical compositions are formulated for oral delivery and may optionally contain inert ingredients such as binders or fillers for the formulation of a unit dosage form, such as a tablet or a capsule. In some embodiments, pharmaceutical compositions are formulated for local administration and may optionally contain inert ingredients such as solvents or emulsifiers for the formulation of a cream, an ointment, a gel, a paste, or an eye drop.

In some embodiments, provided pharmaceutical compositions are sterilized by conventional sterilization techniques, e.g., may be sterile filtered. Resulting aqueous solutions may be packaged for use as is, or lyophilized. Lyophilized preparations can be, for example, combined with a sterile aqueous carrier prior to administration. The pH of preparations typically will be between 3 and 11, more preferably between 5 and 9 or between 6 and 8, and most preferably between 6 and 7, such as 6 to 6.5. Resulting compositions in solid form may be packaged, for example, in multiple single dose units, each containing a fixed amount of the above-mentioned agent or agents, such as in a sealed package of tablets or capsules. Pharmaceutical compositions in solid form can also be packaged in a container for a flexible quantity, such as in a squeezable tube designed for a topically applicable cream or ointment.

### Methods of Treatment

In one aspect, the present disclosure provides methods of treatment comprising administering to a subject in need thereof a compound (e.g., radioimmunoconjugate) as disclosed herein.

### Subjects

In some disclosed methods, a therapy (e.g., comprising a therapeutic agent) is administered to a subject. In some embodiments, the subject is a mammal, e.g., a human.

In some embodiments, the subject has cancer or is at risk of developing cancer. For example, the subject may have been diagnosed with cancer. For example, the cancer may be a primary cancer or a metastatic cancer. Subjects may have any stage of cancer, e.g., stage I, stage II, stage III, or stage IV with or without lymph node involvement and with or without metastases. Provided compounds (e.g., radioimmunoconjugates) and compositions may prevent or reduce further growth of the cancer and/or otherwise ameliorate the cancer (e.g., prevent or reduce metastases). In some embodiments, the subject does not have cancer but has been determined to be at risk of developing cancer, e.g., because of the presence of one or more risk factors such as environmental exposure, presence of one or more genetic mutations or variants, family history, etc. In some embodiments, the subject has not been diagnosed with cancer.

In some embodiments, the cancer is any cancer that comprises cells expressing EGFRvIII. In certain embodiments, the cancer is glioblastoma multiforme or carcinoma.

### Administration and Dosage

Compounds (e.g., radioimmunoconjugates) and pharmaceutical compositions thereof disclosed herein may be administered by any of a variety of routes of administration, including systemic and local routes of administration

Systemic routes of administration include parenteral routes and enteral routes. In some embodiments, compounds (e.g., radioimmunoconjugates) or pharmaceutical compositions thereof are administered by a parenteral route, for example, intravenously, intraarterially, intraperitoneally, subcutaneously, intracranially, or intradermally. In some embodiments, compounds (e.g., radioimmunoconjugates) or pharmaceutical compositions thereof are administered intravenously. In some embodiments, compounds (e.g., radioimmunoconjugates) or pharmaceutical compositions thereof are administered by an enteral route of administration, for example, trans-gastrointestinal, or orally.

Local routes of administration include, but are not limited to, peritumoral injections and intratumoral injections.

Pharmaceutical compositions can be administered for radiation treatment planning, diagnostic, and/or therapeutic treatments. When administered for radiation treatment planning or diagnostic purposes, the compound (e.g., radioimmunoconjugate) may be administered to a subject in a diagnostically effective dose and/or an amount effective to determine the therapeutically effective dose. In therapeutic applications, pharmaceutical compositions may be administered to a subject (e.g., a human) already suffering from a condition (e.g., cancer) in an amount sufficient to cure or at least partially arrest the symptoms of the disorder and its complications. An amount adequate to accomplish this purpose is defined as a “therapeutically effective amount,” an amount of a compound sufficient to substantially improve at least one symptom associated with the disease or a medical condition. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but may, for example, provide a treatment for a disease or condition such that the onset of the disease or condition is delayed, hindered, or prevented, such that the disease or condition symptoms are ameliorated, or such that the term of the disease or condition is changed. For example, the disease or condition may become less severe and/or recovery is accelerated in an individual. In some embodiments, a subject is administered a first dose of a compound (e.g., radioimmunoconjugate) or composition in an amount effective for radiation treatment planning, then administered a second dose or set of doses of the compound (e.g., radioimmunoconjugate) or composition in a therapeutically effective amount.

For treating or preventing cancers that comprises cells expressing EGFRVIII, the method typically comprises administering to a subject (e.g., a human) in need thereof a first dose of a compound or composition provided above in an amount effective for radiation treatment planning, followed by administering subsequent doses of a compound or composition provided above in a therapeutically effective amount.

In some embodiments, the compound or composition administered in the first dose and the compound or composition administered in the second dose are the same.

In some embodiments, the compound or composition administered in the first dose and the compound or composition administered in the second dose are different.

Therapeutically effective amounts may depend on the severity of the disease or condition and other characteristics of the subject (e.g., weight). Therapeutically effective amounts of disclosed compounds (e.g., radioimmunoconjugates) and compositions for subjects (e.g., mammals such as humans) can be determined by the ordinarily-skilled artisan with consideration of individual differences (e.g., differences in age, weight and the condition of the subject).

In some embodiments, disclosed compounds (e.g., radioimmunoconjugates) exhibit an enhanced ability to target cancer cells. In some embodiments, the effective amount of disclosed compounds (e.g., radioimmunoconjugates) is lower than (e.g., less than or equal to about 90%, 75%, 50%, 40%, 30%, 20%, 15%, 12%, 10%, 8%, 7%, 6%, 5%, 4%, 3%, 2%, 1%, 0.5%, or 0.1% of) the equivalent dose for a therapeutic effect of the unconjugated, and/or non-radiolabeled targeting moiety.

Single or multiple administrations of compounds or pharmaceutical compositions disclosed herein including an effective amount can be carried out with dose levels and pattern being selected by the treating physician. Dose and administration schedule can be determined and adjusted based on the severity of the disease or condition in the subject, which may be monitored throughout the course of treatment according to the methods commonly practiced by clinicians or those described herein.

### Other Agents

In some embodiments, disclosed methods further include administering an antiproliferative agent, radiation sensitizer, or an immunoregulatory or immunomodulatory agent.

By “antiproliferative” or “antiproliferative agent,” as used interchangeably herein, is meant any anticancer agent, including those antiproliferative agents listed in Table 2, any of which can be used in combination with a radioimmunoconjugate as described herein to treat a condition or disorder. An exemplary antiproliferative agent used in the methods of this disclosure is temozolomide (TMZ), which can be administered in combination with a radioimmunoconjugate set forth above, in the presence or absence of external beam radiation. Antiproliferative agents also include organo-platinum derivatives, naphtoquinone and benzoquinone derivatives, chrysophanic acid and anthroquinone derivatives thereof.

By “immunoregulatory agent” or “immunomodulatory agent,” as used interchangeably herein, is meant any immuno-modulator, including those listed in Table 2, any of which can be used in combination with a radioimmunoconjugate.

As used herein, “radiation sensitizer” includes any agent that increases the sensitivity of cancer cells to radiation therapy. Radiation sensitizers may include, but are not limited to, 5-fluorouracil, analogs of platinum (e.g., cisplatin, carboplatin, oxaliplatin), gemcitabine, EGFR antagonists (e.g., cetuximab, gefitinib), farnesyltransferase inhibitors, COX-2 inhibitors, bFGF antagonists, and VEGF antagonists.

TABLE 2

| | | |
|---|---|---|
| Alkylating agents | Busulfan | Chlorambucil |
| | dacarbazine | procarbazine |
| | ifosfamide | altretamine |
| | hexamethylmelamine | estramustine phosphate |
| | thiotepa | mechlorethamine |
| | dacarbazine | streptozocin |
| | lomustine | temozolomide |
| | cyclophosphamide | Semustine |
| Platinum agents | spiroplatin | lobaplatin (Aeterna) |
| | tetraplatin | satraplatin (Johnson Matthey) |
| | ormaplatin | BBR-3464 (Hoffmann-La |
| | iproplatin | Roche) |
| | picoplatin | Miriplatin |
| | oxaliplatin | AP-5280 (Access) |
| | carboplatin | cisplatin |
| Antimetabolites | azacytidine | trimetrexate |
| | Floxuridine | deoxycoformycin |
| | 2-chlorodeoxyadenosine | pentostatin |
| | 6-mercaptopurine | hydroxyurea |
| | 6-thioguanine | decitabine (SuperGen) |
| | cytarabine | clofarabine (Bioenvision) |
| | 2-fluorodeoxy cytidine | irofulven (MGI Pharma) |
| | methotrexate | DMDC (Hoffmann-La Roche) |
| | tomudex | ethynylcytidine (Taiho) |
| | fludarabine | gemcitabine |
| | raltitrexed | capecitabine |
| Topoisomerase | amsacrine | exatecan mesylate (Daiichi) |
| inhibitors | epirubicin | quinamed (ChemGenex) |
| | etoposide | gimatecan (Sigma-Tau) |
| | teniposide or mitoxantrone | diflomotecan (Beaufour-Ipsen) |
| | 7-ethyl-10-hydroxy- | TAS-103 (Taiho) |
| | camptothecin | elsamitrucin (Spectrum) |
| | dexrazoxanet (TopoTarget) | Edotecarin |
| | pixantrone (Novuspharma) | Cositecan |
| | rebeccamycin analogue | Belotecan |
| | (Exelixis) | hydroxycamptothecin (SN-38) |
| | BBR-3576 (Novuspharma) | |
| | rubitecan (SuperGen) | |
| | irinotecan (CPT-11) | |
| | topotecan | |
| Antitumor | valrubicin | azonafide |
| antibiotics | therarubicin | anthrapyrazole |
| | idarubicin | oxantrazole |
| | rubidazone | losoxantrone |
| | plicamycin | Sabarubicin |
| | porfiromycin | |
| | mitoxantrone (novantrone) | Epirubicin |
| | amonafide | mitoxantrone |
| | | doxorubicin |
| Antimitotic | colchicine | E7010 (Abbott) |
| agents | vinblastine | PG-TXL (Cell Therapeutics) |
| | vindesine | IDN 5109 (Bayer) |
| | dolastatin 10 (NCI) | A 105972 (Abbott) |
| | rhizoxin (Fujisawa) | A 204197 (Abbott) |
| | mivobulin (Warner-Lambert) | LU 223651 (BASF) |
| | cemadotin (BASF) | D 24851 (ASTAMedica) |
| | RPR 109881A (Aventis) | ER-86526 (Eisai) |
| | TXD 258 (Aventis) | combretastatin A4 (BMS) |
| | epothilone B (Novartis) | isohomohalichondrin-B |
| | T 900607 (Tularik) | (PharmaMar) |
| | T 138067 (Tularik) | ZD 6126 (AstraZeneca) |
| | cryptophycin 52 (Eli Lilly) | AZ10992 (Asahi) |
| | vinflunine (Fabre) | IDN-5109 (Indena) |
| | auristatin PE (Teikoku Hormone) | AVLB (Prescient NeuroPharma) |
| | BMS 247550 (BMS) | azaepothilone B (BMS) |
| | BMS 184476 (BMS) | BNP-7787 (BioNumerik) |
| | BMS 188797 (BMS) | CA-4 prodrug (OXiGENE) |
| | taxoprexin (Protarga) | dolastatin-10 (NIH) |
| | SB 408075 (GlaxoSmithKline) | CA-4 (OXiGENE) |
| | Vinorelbine | docetaxel |
| | Trichostatin A | vincristine |
| | | paclitaxel |
| Aromatase | aminoglutethimide | YM-511 (Yamanouchi) |
| inhibitors | atamestane (BioMedicines) | formestane |
| | letrozole | exemestane |
| | anastrazole | |
| Thymidylate | pemetrexed (Eli Lilly) | nolatrexed (Eximias) |
| synthase inhibitors | ZD-9331 (BTG) | CoFactor ™ (BioKeys) |
| DNA antagonists | trabectedin (PharmaMar) | edotreotide (Novartis) |
| | glufosfamide (Baxter | mafosfamide (Baxter |
| | International) | International) |
| | albumin + 32P (Isotope | apaziquone (Spectrum |
| | Solutions) | Pharmaceuticals) |
| | thymectacin (NewBiotics) | O6 benzyl guanine (Paligent) |
| Farnesyltransferase | arglabin (NuOncology Labs) | tipifarnib (Johnson & Johnson) |
| inhibitors | lonafarnib (Schering-Plough) | perillyl alcohol (DOR |
| | BAY-43-9006 (Bayer) | BioPharma) |
| Pump inhibitors | CBT-1 (CBA Pharma) | zosuquidar trihydrochloride (Eli |
| | tariquidar (Xenova) | Lilly) |
| | MS-209 (Schering AG) | biricodar dicitrate (Vertex) |
| Histone | tacedinaline (Pfizer) | pivaloyloxymethyl butyrate |
| acetyltransferase | SAHA (Aton Pharma) | (Titan) |
| inhibitors | MS-275 (Schering AG) | depsipeptide (Fujisawa) |
| Metalloproteinase | Neovastat (Aeterna Laboratories) | CMT-3 (CollaGenex) |
| inhibitors | marimastat (British Biotech) | BMS-275291 (Celltech) |
| Ribonucleoside | gallium maltolate (Titan) | tezacitabine (Aventis) |
| reductase inhibitors | triapine (Vion) | didox (Molecules for Health) |
| TNF alpha | virulizin (Lorus | revimid (Celgene) |
| agonists/antagonists | Therapeutics) | |
| | CDC-394 (Celgene) | |
| Endothelin A | atrasentan (Abbott) | YM-598 (Yamanouchi) |
| receptor antagonist | ZD-4054 (AstraZeneca) | |
| Retinoic acid | fenretinide (Johnson & Johnson) | alitretinoin (Ligand) |
| receptor agonists | LGD-1550 (Ligand) | |
| Immuno- | interferon | dexosome therapy (Anosys) |
| modulators | oncophage (Antigenics) | pentrix (Australian Cancer |
| | GMK (Progenics) | Technology) |
| | adenocarcinoma vaccine | ISF-154 (Tragen) |
| | (Biomira) | cancer vaccine (Intercell) |
| | CTP-37 (AVI BioPharma) | norelin (Biostar) |
| | IRX-2 (Immuno-Rx) | BLP-25 (Biomira) |
| | PEP-005 (Peplin Biotech) | MGV (Progenics) |
| | synchrovax vaccines (CTL | β-alethine (Dovetail) |
| | Immuno) | CLL therapy (Vasogen) |
| | melanoma vaccine (CTL | Ipilimumab (BMS), |
| | Immuno) | CM-10 (cCam Biotherapeutics) |
| | p21 RAS vaccine (GemVax) | atezolizumab (Genentech) |
| | MAGE-A3 (GSK) | |
| | nivolumab (BMS) | |
| | abatacept (BMS) | |
| | pembrolizumab (Merck) | |
| Hormonal and | estrogens | dexamethasone |
| antihormonal | conjugated estrogens | prednisone |
| agents | ethinyl estradiol | methylprednisolone |
| | chlortrianisen | prednisolone |
| | idenestrol | aminoglutethimide |
| | hydroxyprogesterone caproate | leuprolide |
| | medroxyprogesterone | octreotide |
| | testosterone | mitotane |
| | testosterone propionate; | P-04 (Novogen) |
| | fluoxymesterone | 2-methoxyestradiol (EntreMed) |
| | methyltestosterone | arzoxifene (Eli Lilly) |
| | diethylstilbestrol | tamoxifen |
| | megestrol | toremofine |
| | bicalutamide | goserelin |
| | flutamide | Leuporelin |
| | nilutamide | bicalutamide |
| Photodynamic | talaporfin (Light Sciences) | Pd-bacteriopheophorbide (Yeda) |
| agents | Theralux (Theratechnologies) | Motexafin lutetium |
| | motexafin gadolinium | hypericin |
| | (Pharmacyclics) | |
| Kinase Inhibitors | imatinib (Novartis) | EKB-569 (Wyeth) |
| | leflunomide (Sugen/Pharmacia) | kahalide F (PharmaMar) |
| | ZD1839 (AstraZeneca) | CEP-701 (Cephalon) |
| | erlotinib (Oncogene Science) | CEP-751 (Cephalon) |
| | canertinib (Pfizer) | MLN518 (Millenium) |
| | squalamine (Genaera) | PKC412 (Novartis) |
| | SU5416 (Pharmacia) | Phenoxodiol (Novogen) |
| | SU6668 (Pharmacia) | C225 (ImClone) |
| | ZD4190 (AstraZeneca) | rhu-Mab (Genentech) |
| | ZD6474 (AstraZeneca) | MDX-H210 (Medarex) |
| | vatalanib (Novartis) | 2C4 (Genentech) |
| | PKI166 (Novartis) | MDX-447 (Medarex) |
| | GW2016 (GlaxoSmithKline) | ABX-EGF (Abgenix) |
| | EKB-509 (Wyeth) | IMC-1C11 (ImClone) |
| | trastuzumab (Genentech) | Tyrphostins |
| | OSI-774 (Tarceva ™) | Gefitinib (Iressa) |
| | CI-1033 (Pfizer) | PTK787 (Novartis) |
| | SU11248 (Pharmacia) | EMD 72000 (Merck) |
| | RH3 (York Medical) | Emodin |
| | Genistein | Radicinol |
| | Radicinol | Vemurafenib (B-Raf enzyme |
| | Met-MAb (Roche) | inhibitor, Daiichi Sankyo) |
| | SR-27897 (CCK A inhibitor, Sanofi- | ceflatonin (apoptosis promotor, |
| | Synthelabo) | ChemGenex) |
| | tocladesine (cyclic AMP agonist, Ribapharm) | BCX-1777 (PNP inhibitor, BioCryst) |
| | alvocidib (CDK inhibitor, Aventis) | ranpirnase (ribonuclease stimulant, |
| | CV-247 (COX-2 inhibitor, Ivy Medical) | Alfacell) |
| | P54 (COX-2 inhibitor, Phytopharm) | galarubicin (RNA synthesis inhibitor, |
| | CapCell ™ (CYP450 stimulant, Bavarian | Dong-A) |
| | Nordic) | tirapazamine (reducing agent, SRI |
| | GCS-100 (gal3 antagonist, GlycoGenesys) | International) |
| | G17DT immunogen (gastrin inhibitor, | N-acetylcysteine (reducing agent, |
| | Aphton) | Zambon) |
| | efaproxiral (oxygenator, Allos Therapeutics) | R-flurbiprofen (NF-kappaB inhibitor, |
| | PI-88 (heparanase inhibitor, Progen) | Encore) |
| | tesmilifene (histamine antagonist, YM | 3CPA (NF-kappaB inhibitor, Active |
| | BioSciences) | Biotech) |
| | histamine (histamine H2 receptor agonist, | seocalcitol (vitamin D receptor agonist, |
| | Maxim) | Leo) |
| | tiazofurin (IMPDH inhibitor, Ribapharm) | 131-I-TM-601 (DNA antagonist, |
| | cilengitide (integrin antagonist, Merck KGaA) | TransMolecular) |
| | SR-31747 (IL-1 antagonist, Sanofi- | eflornithine (ODC inhibitor, ILEX |
| | Synthelabo) | Oncology) |
| | CCI-779 (mTOR kinase inhibitor, Wyeth) | minodronic acid (osteoclast inhibitor, |
| | exisulind (PDE V inhibitor, Cell Pathways) | Yamanouchi) |
| | CP-461 (PDE V inhibitor, Cell Pathways) | indisulam (p53 stimulant, Eisai) |
| | AG-2037 (GARFT inhibitor, Pfizer) | aplidine (PPT inhibitor, PharmaMar) |
| | WX-UK1 (plasminogen activator inhibitor, | gemtuzumab (CD33 antibody, Wyeth |
| | Wilex) | Ayerst) |
| | PBI-1402 (PMN stimulant, ProMetic | PG2 (hematopoiesis enhancer, |
| | LifeSciences) | Pharmagenesis) |
| | bortezomib (proteasome inhibitor, | Immunol ™ (triclosan oral rinse, Endo) |
| | Millennium) | triacetyluridine (uridine prodrug, |
| | SRL-172 (T cell stimulant, SR Pharma) | Wellstat) |
| | TLK-286 (glutathione S transferase inhibitor, | SN-4071 (sarcoma agent, Signature |
| | Telik) | BioScience) |
| | PT-100 (growth factor agonist, Point | TransMID-107 ™ (immunotoxin, KS |
| | Therapeutics) | Biomedix) |
| | midostaurin (PKC inhibitor, Novartis) | PCK-3145 (apoptosis promotor, |
| | bryostatin-1 (PKC stimulant, GPC Biotech) | Procyon) |
| | CDA-II (apoptosis promotor, Everlife) | doranidazole (apoptosis promotor, Pola) |
| | SDX-101 (apoptosis promotor, Salmedix) | CHS-828 (cytotoxic agent, Leo) |
| | rituximab (CD20 antibody, Genentech | trans-retinoic acid (differentiator, NIH) |
| | carmustine | MX6 (apoptosis promotor, MAXIA) |
| | Mitoxantrone | apomine (apoptosis promotor, ILEX |
| | Bleomycin | Oncology) |
| | Absinthin | urocidin (apoptosis promotor, Bioniche) |
| | Chrysophanic acid | Ro-31-7453 (apoptosis promotor, La |
| | Cesium oxides | Roche) |
| | BRAF inhibitors, | brostallicin (apoptosis promotor, |
| | PD-L1 inhibitors | Pharmacia) |
| | MEK inhibitors | β-lapachone |
| | bevacizumab | gelonin |
| | angiogenesis inhibitors | cafestol |
| | dabrafenib | kahweol |
| | | caffeic acid |
| | | Tyrphostin AG |
| | | PD-1 inhibitors |
| | | CTLA-4 inhibitors |
| | | sorafenib |

The following specific examples are to be construed as merely illustrative, and not limitative of the remainder of the disclosure in any way whatsoever.

### EXAMPLES

### Example 1. General Materials and Methods

Lutetium-177 can be obtained from ITM Medical Isotopes as lutetium trichloride in a 0.05 N hydrochloric acid solution; indium-111, as indium trichloride in a 0.05 N hydrochloric acid solution, can be obtained from BWXT; and actinium-225 can be obtained as actinium-225 trinitrate from Oak Ridge National Laboratories.

Analytical HPLC-MS can be performed using a Waters Acquity HPLC-MS system comprised of a Waters Acquity Binary Solvent Manager, a Waters Acquity Sample Manager (samples cooled to 10° C.), a Water Acquity Column Manager (column temperature 30° C.), a Waters Acquity Photodiode Array Detector (monitoring at 254 nm and 214 nm), a Waters Acquity TQD with electrospray ionization and a Waters Acquity BEH C18, 2.1×50 (1.7 μm) column. Preparative HPLC can be performed using a Waters HPLC system comprised of a Waters 1525 Binary HPLC pump, a Waters 2489 UV/Visible Detector (monitoring at 254 nm and 214 nm) and a Waters XBridge Prep phenyl or C18 19×100 mm (5 μm) column.

HPLC elution method 1: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; initial=90% A, 3-3.5 min=0% A, 4 min=90% A, 5 min=90% A.

HPLC elution method 2: Waters XBridge Prep Phenyl 19×100 mm (5 μm) column; mobile phase A: H₂O (0.1% v/v TFA); mobile phase B: acetonitrile (0.1% v/v TFA); flow rate: 10 mL/min; initial=80% A, 13 min=0% A.

HPLC elution method 3: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; initial=90% A, 8 min=0% A, 10 min=0% A, 11 min=90% A, 12 min=90% A.

HPLC elution method 4: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=80% A, 3 min=80% A, 13 min=20% A, 18 min=0% A.

HPLC elution method 5: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min: initial=90% A, 3 min=90% A, 13 min=0% A, 20 min=0% A.

HPLC elution method 6: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=75% A, 13 min=0% A, 15 min=0% A.

HPLC elution method 7: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=80% A, 12 min=0% A, 15 min=0% A.

HPLC elution method 8: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=90% A, 12 min=0% A, 15 min=0% A.

Analytical Size Exclusion Chromatography (SEC) can be performed using a Waters system comprised of a Waters 1525 Binary HPLC pump, a Waters 2489 UV/Visible Detector (monitoring at 280 nm), a Bioscan Flow Count radiodetector (FC-3300) and TOSOH TSKgel G3000SW×1, 7.8×300 mm column. The isocratic SEC method can have a flow rate of, e.g., mL/min, with a mobile phase of 0.1 M phosphate, 0.6 M NaCl, 0.025% sodium azide, pH=7.

MALDI-MS (positive ion) can be performed using a MALDI Bruker Ultraflextreme Spectrometer.

Radio thin-layer chromatography (radio-TLC) can be performed with Bioscan AR-2000 Imaging Scanner, and can be carried out on iTLC-SG glass microfiber chromatography paper (Agilent Technologies, SGI0001) plates using citrate buffer (0.1 M, pH 5.5).

Generation and evaluation of certain EGFRvIII monoclonal antibodies can be referred to WO2020191485A1 (which is incorporated by reference in its entirety).

### Example 2. Synthesis of 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B)

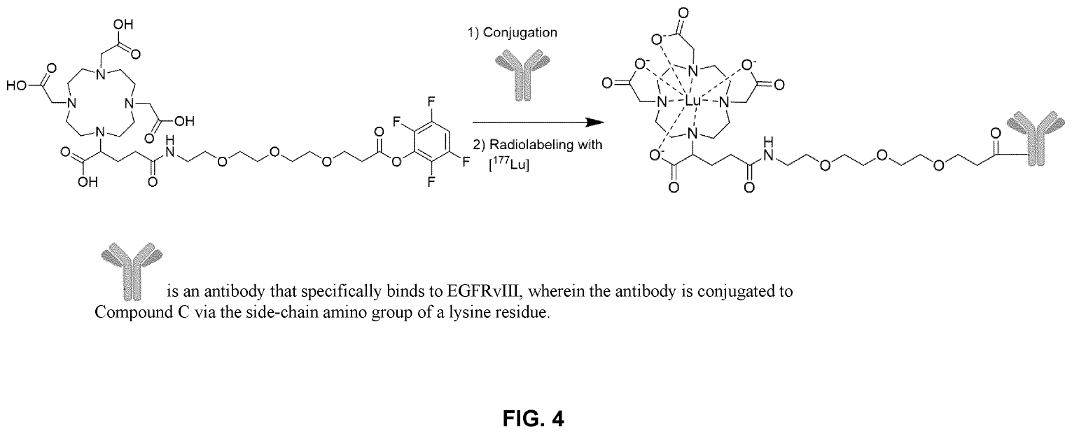

A bifunctional chelate, 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B), can be synthesized according to the scheme provided in FIG. 2. To a solution of 5-(tert-butoxy)-5-oxo-4-(4,7,10-tris(2-(tert-butoxy)-2-oxoethyl)-1,4,7,10-tetraazacyclododecan-1-yl) pentanoic acid (DOTA-GA-(tBu) 4, 50 mg, 0.07 mmol) in ACN (2.0 mL), DSC (50 mg, 0.21 mmol) is added, followed by pyridine, (0.20 mL, 2.48 mmol). The reaction is stirred at room temperature for 1 hour. To the reaction mixture is added 11-aminoundecanoic acid, (70 mg, 0.36 mmol) followed by PBS solution (1.0 mL) at room temperature. The reaction is stirred for 72 hours at room temperature. The reaction mixture is filtered with a syringe filter and purified directly by Prep-HPLC using method 6 to yield Intermediate 2-A.

To a solution of Intermediate 2-A (40 mg, 0.03 mmol), TFP (90 mg, 0.54 mmol) and EDC (40 mg, 0.27 mmol) in ACN (1.0 mL) is added pyridine (0.05 mL, 50 mg, 0.62 mmol) at room temperature. The solution is stirred at room temperature for 24 hours. The reaction is purified directly by Prep-HPLC using method 7 to provide Intermediate 2-B as a wax after concentration using a Biotage V10 Rapid Evaporator.

Intermediate 2-B is dissolved in DCM/TFA (1.0 mL/2.0 mL) and allowed to stir at room temperature for 24 hours. The reaction is concentrated by air stream and purified directly by Prep-HPLC using method 8 to yield Compound B as a clear wax after concentration. An aliquot is analyzed by HPLC-MS elution method 3.

¹H NMR (600 MHZ, DMSO-d₆) δ 7.99-7.88 (m, 1H), 7.82 (t, J=5.5 Hz, 1H), 3.78 (broad s, 4H), 3.43 (broad s, 12H), 3.08 (broad s, 4H), 3.00 (m, 3H), 2.93 (broad s, 3H), 2.77 (t, J=7.2 Hz, 2H), 2.30 (broad s, 2H), 1.88 (broad s, 2H), 1.66 (p, J=7.3 Hz, 2H), 1.36 (m, 4H), 1.32-1.20 (m, 9H).

### Example 3. Synthesis of 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (Compound C)

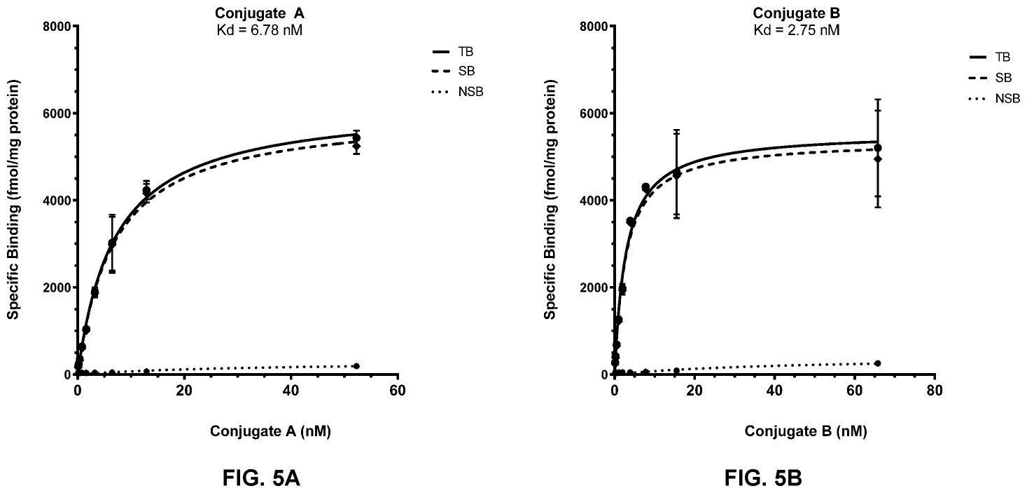

A bifunctional chelate, 4-{[2-(2-{2-[3-oxo-3-(2,3,5,6-tetrafluorophenoxy) propoxy]ethoxy}ethoxy)ethyl]carbamoyl}-2-[4,7,10-tris(carboxymethyl)-1,4,7,10-tetraazacyclododecan-1-yl]butanoic acid (Compound C), is synthesized according to the scheme provided in FIG. 3.

To a solution of 5-(tert-butoxy)-5-oxo-4-(4,7,10-tris(2-(tert-butoxy)-2-oxoethyl)-1,4,7,10-tetraazacyclododecan-1-yl) pentanoic acid (DOTA-GA (tBu)₄, 100 mg, 0.143 mmol) in ACN (8.0 mL) is added DSC (73 mg, 0.285 mmol) and pyridine (0.80 mL, 9.89 mmol). The reaction mixture is stirred for 90 min at ambient temperature. This solution is added to a semi-solution of amino-PEG3-acid (63 mg, 0.285 mmol in 1.2 mL of DMF) in a 100 mL round bottom flask. After 4 hours at ambient temperature, the reaction is worked up by concentrating to dryness under a stream of air. The crude material is purified by HPLC elution method 2 (dissolved the crude in 6 mL of 20% ACN/H₂O). The fractions containing product are pooled and concentrated under vacuum and then co-evaporated with ACN (3×2 mL).

To a vial containing Intermediate 1-A (82 mg, 60 umol) is added ACN (2 mL), NEt₃ (50 μL, 360 μmol, 6 equiv.), HBTU (23 mg, 60 μmol, 1 equiv) and a TFP solution (50 mg, 300 μmol, 5 equiv., dissolved in 250 μL of ACN). The resulting clear solution is stirred at ambient temperature for 3 hours. The reaction is worked up by concentrating the solution to dryness under an air stream and is then diluted with ACN/H₂O (1:1, 3 mL total) and purified on preparative HPLC using elution method 4. Fractions containing product are pooled and concentrated under vacuum and then co-evaporated with ACN (3×2 mL). Intermediate 1-B is obtained as a clear residue.

To a vial containing Intermediate 1-B (67 mg, 64 μmol) is added DCM (2 mL) and TFA (2 mL). The resulting solution is stirred at ambient temperature for 16 hour. Additional, TFA (2 mL) is added, and the reaction is stirred at ambient temperature for 6 hours. The reaction is concentrated to dryness under an air stream, with the crude product being finally dissolved in ACN/H₂O (1 mL of 10% ACN/H₂O). The crude reaction solution is then purified by preparative HPLC using elution method 5. The fractions containing product are pooled, frozen and lyophilized. Compound C is obtained as a white solid. An aliquot is analyzed by HPLC-MS elution method 3.

¹H NMR (DMSO-d₆, 600 MHZ) δ 7.97-7.91 (m, 2H), 3.77 (t, 2H, J=6.0 Hz), 3.58-3.55 (m, 2H), 3.53-3.48 (m, 8H), 3.44-3.38 (m, 10H), 3.23-3.08 (m, 11H), 3.02 (t, 2H, J=6.0 Hz), 2.93 (broad s, 4H), 2.30 (broad s, 2H), 1.87 (broad s, 2H).

### Example 4. Conjugation and Radiolabeling for Synthesis of [¹⁷⁷Lu]-Compound C-Anti-EGFRvIII Conjugates (Conjugate A and Conjugate B)

¹¹⁷Lu-radiolabeled Compound C-anti-EGFRvIII conjugates may be prepared as follows: Compound C (1.4 μmole) is dissolved in a hydrochloric acid solution (0.001 M). An aliquot of Compound C solution (19 μL, 90 nmole) is added to a solution containing anti-EGFRvIII antibody (1.8 nmole) in a carbonate buffer (pH 9.5). After 1 hour at ambient temperature, the resulting immunoconjugate is purified via a Sephadex G-50 resin packed column. The immunoconjugate Compound C-anti-EGFRvIII is eluted from the column with acetate buffer (pH 6.5). Identities of eluates can be confirmed by, e.g., MALDI-TOF, where typical chelate to antibody ratios (CARs) of 3-4 are obtained.

¹⁷⁷Lu (1.6 mCi, 3.9 μL) is added to a solution of Compound C-anti-EGFRvIII (150 μg in acetate buffer (pH 6.5)). After 40 minutes at ambient temperature, the crude product, [¹⁷⁷Lu]-Compound C-anti-EGFRvIII, is purified via a Sephadex G-50 resin packed column eluted with acetate buffer.

Two [¹⁷⁷Lu]-Compound C-anti-EGFRvIII conjugates, i.e., Conjugates A and B, were prepared using two anti-EGFRvIII antibodies. More specifically, Conjugate A was prepared using EGFRVIII antibody hH2-hL3, comprising a light chain region having the amino acid sequence set forth in SEQ ID NO: 182 and a heavy chain region having the amino acid sequence set forth in SEQ ID NO: 184; Conjugate B was prepared using EGFRvIII antibody hH3-hL1, comprising a light chain region having the amino acid sequence set forth in SEQ ID NO: 180 and a heavy chain region having the amino acid sequence set forth in SEQ ID NO: 185. Conjugates A and B were prepared with chemical purities>99%, radiochemical purities>99%, and specific activities between 7-10 mCi/mg.

### Example 5. Radiosynthesis of [¹¹¹In]- and [²²⁵Ac]-radioimmunoconjugates

### Radiosynthesis of Radioimmunoconjugate [¹¹¹In]-Compound C-Anti-EGFRviII

A 1.5 mL Eppendorf tube was charged with a SABST solution of Compound C-anti-EGFRvIII immunoconjugate (0.0492 mL of 3.05 mg/mL, i.e., 0.150 mg; prepared from EGFRvIII antibody hH3-hL1 following protocols similar to those described in Example 4 above), additional SABST to make a 2.0 mg/mL solution, and a 0.05±0.01 M HCl solution of [¹¹¹In]Cl₃ (2.0 mCi). After 60 minutes at 37° C., radio-TLC analysis of the reaction mixture (iTLC-SG plates, 5% methanol in 0.02 M citrate buffer as the mobile phase) indicated a radiochemical conversion (RCC) of 97%. Purification was carried out using a 1 mL column packed with Sephadex G50 resin (hydrated with SABST). The product fractions were eluted using SABST and combined. SABST solutions of sodium L-ascorbate and diethylenetriamine-pentaacetic acid calcium trisodium salt hydrate (DTPA) were added to give a final formulation of 10 mM ascorbate and 1 mM DTPA. Analysis of the resulting formulation by radio-TLC and SEC-HPLC at end-of-synthesis (EOS) indicated the formation of [¹¹¹In]-Compound C-anti-EGFRvIII (364 μL, 0.299 mg/mL, 8.9 mCi/mg specific activity, 99% radiochemical purity, and >95% chemical purity).

### Radiosynthesis of Radioimmunoconjugate [¹¹¹In]-Compound C-IgG4

A 1.5 mL Eppendorf tube was charged with a SABST solution of Compound C-IgG4 immunoconjugate (0.0318 mL of 4.72 mg/mL, i.e., 0.150 mg; prepared from isotype IgG4 antibody S228P (purchased from Bio X Cell, Cat #BE0349-5 MG-R) following similar protocols to those described in Example 4 above), additional SABST to make a 2.0 mg/mL solution and a 0.05±0.01 M HCl solution of [¹¹¹In]C₁; (2.25 mCi). After 60 minutes at room temperature, radio-TLC analysis of the reaction mixture (iTLC-SG plates, 5% methanol in 0.02 M citrate buffer as the mobile phase) indicated a radiochemical conversion (RCC) of 99%. Purification was carried out using a 1 mL column packed with Sephadex G50 resin (hydrated with SABST). The product fractions were eluted using SABST and combined. SABST solutions of sodium L-ascorbate and diethylenetriamine-pentaacetic acid calcium trisodium salt hydrate (DTPA) were added to give a final formulation of 10 mM ascorbate and 1 mM DTPA. Analysis of the resulting formulation by radio-TLC and SEC-HPLC at end-of-synthesis (EOS) indicated the formation of [¹¹¹In]-Compound C-IgG4 (364 μL, 0.362 mg/mL, 9.3 mCi/mg specific activity, 99% radiochemical purity, and >95% chemical purity).

### Radiosynthesis of Radioimmunoconjugate [²²⁵Ac]-Compound C-Anti-EGFRvIII

A 1.5 mL Eppendorf tube was charged with a SABST solution of Compound C-anti-EGFRvIII immunoconjugate (0.0656 mL of 3.05 mg/mL, i.e., 0.200 mg; prepared from anti-EGFRvIII antibody hH3-hL1 following the protocols in Example 4 above), additional SABST to make a 2.0 mg/mL solution and a 0.001 M HCl solution of [²²⁵Ac]Cl₃ (10.9 μCi). After 2 hours at 37° C., radio-TLC analysis of the reaction mixture (iTLC-SG plates, 5% methanol in 0.02 M citrate buffer as the mobile phase) indicated a radiochemical conversion (RCC) of 99% (the plate was scanned on the radio-TLC scanner the following day). Purification was carried out using a 1 mL column packed with Sephadex G50 resin (hydrated with SABST). The product fractions were eluted using SABST and combined. SABST solutions of sodium L-ascorbate and diethylenetriamine-pentaacetic acid calcium trisodium salt hydrate (DTPA) were added to give a final formulation of 10 mM ascorbate and 1 mM DTPA. Analysis of the resulting formulation by radio-TLC and SEC-HPLC at end-of-synthesis (EOS) indicated the formation of [²²⁵Ac]-Compound C-anti-EGFRVIII (461 μL, 0.406 mg/mL, 0.0454 mCi/mg specific activity, 99% radiochemical purity, and >95% chemical purity).

### Radiosynthesis of Radioimmunoconjugate [²²⁵Ac]-Compound C-IgG4

A 1.5 mL Eppendorf tube was charged with a SABST solution of IgG4 immunoconjugate (0.0847 mL of 4.72 mg/mL, i.e., 0.400 mg; prepared from isotype IgG4 antibody S228P following protocols similar to those described in Example 4 above), additional SABST to make a 2.0 mg/mL solution and a 0.001 M HCl solution of [225Ac]Cl₃ (21.1 μCi). After 2 hours at 37° C., radio-TLC analysis of the reaction mixture (iTLC-SG plates, 5% methanol in 0.02 M citrate buffer as the mobile phase) indicated a radiochemical conversion (RCC) of 68% (the plate was scanned on the radio-TLC scanner the following day). Purification was carried out using a 1 mL column packed with Sephadex G50 resin (hydrated with SABST). The product fractions were eluted using SABST and combined. SABST solutions of sodium L-ascorbate and diethylenetriamine-pentaacetic acid calcium trisodium salt hydrate (DTPA) were added to give a final formulation of 10 mM ascorbate and 1 mM DTPA. Analysis of the resulting formulation by radio-TLC and SEC-HPLC at end-of-synthesis (EOS) indicated the formation of [²²⁵Ac]-Compound C-IgG4 (463 μL, 0.773 mg/mL, 0.0427 mCi/mg specific activity, 99% radiochemical purity, and 95% chemical purity).

### Example 6. In Vitro Binding of [177Lu]-Compound C-Anti-EGFRvIII Conjugates

A study was conducted to evaluate the receptor binding affinity of [¹⁷⁷Lu]-Compound C-anti-EGFRvIII conjugates, i.e., Conjugates A and B, with the U87-EGFRvIII glioblastoma cell line over-expressing EGFRvIII (obtained from National Research Council of Canada).

The materials used in this study are summarized in Table 3 below.

TABLE 3
Materials

| | MW | | | | |
|---|---|---|---|---|---|
| Name | or amount | | Company | Catalog No. | Characteristics |
| Conjugate A | 144 | kDa | | | |
| Conjugate B | 144 | kDa | | | |
| PBS | | | Invitrogen | | pH 7.4 |
| BSA | ~66 | kDa | Sigma | A7906 | pH 6.5-7.5/1% in 0.15M |
| | | | | | NaCl |
| FBS | 500 | ml | Invitrogen | 12483-020 | Qualified, Canada origin |
| Trypsin/EDTA | | | Sigma | T4049 | 0.25%, 0.2 g EDTA |
| Trypan blue | 960.81 | | Sigma | T8154 | Sterile-filtered, cell |
| | | | | | culture tested |
| RPMI | 500 | ml | Invitrogen | 11875-093 | With L-glutamine, |
| | | | | | Phenol Red, |
| | | | | | Without HEPES |
| DMEM | 500 | ml | Invitrogen | | |

| | | | | |
|---|---|---|---|---|
| Triton X-100 | Ave. 625 | Sigma | T8787 | Molecular biology grade |

### Buffers:

1. Cell lysate buffer was prepared using PBS with 1% Triton X-100
2. Cell binding buffer was prepared using PBS with 0.5% BSA

This study followed the procedures described below:
1. Cell preparation:
   a. Harvest attached cells: recover sufficient, U87-EGFRvIII cells from T225 flasks using trypsin/EDTA and transfer to a 50 ml culture tube. Dispense a 50 μl sample of resuspended cells into an Eppendorf micro-tube and dilute with 50 μl of trypan blue. Fill a hemocytometer (10 μl) chamber and count the number of cells present.
      i. Count cells in four quadrants of the counting area.
      ii. Calculate cells/mL: count/4 quadrants×dilution factor×10⁴=cells/mL.
   b. Resuspend cells in medium to a final concentration of 3×10⁵ cells/ml.
   c. Plate 3×10⁵/1 mL U87-EGFRvIII into designated well of the 24-well plates and gently tap to evenly distribute cells (total 6 plates prepared-4 U87-EGFRvIII, 2 blanks)
   d. Incubate plates overnight at 37° C. and 5% CO₂.
2. Binding assay:
   Blocking agent was prepared as follows: 4500 μl as concentration of 4 μM; Final concentration of blocker 2 μM.
      For Conjugate A
         stock=7.58 mg/mL
         mg/ml=52.64 μM, add 342 μl
         Binding buffer: 4158 μl
      For Conjugate B
         stock=6.7 mg/mL
         mg/ml=46.53 μM, add 387 μl
         Binding buffer: 4113 μl
      a. Remove medium from two plates
      b. Wash plates once with 1 mL PBS
      c. Add 200 μl binding buffer into TB well and blank well.
      d. Add 200 μl blocking agent into NSB well and incubate 1 h on ice
         Hot article preparation:
      i. Prepare 4000 μl 160 nM Conjugate A (Tube 1) from stock solution (0.390 mg/ml=2.71 μM) using binding buffer
         Conjugate A: 236.2 μl
         Binding buffer:
         3763.8 μl
         0.2×-dilution of Tube 1 (total 8 dilutions)
      ii. Prepare 4000 μl 160 nM Conjugate B (Tube 1) from stock solution (0.341 mg/ml=2.37 M) using binding buffer
         Conjugate B: 270 μl
         Binding buffer: 3730 μl
         2×-dilution of Tube 1 (total 8 dilutions)
      e. Add 200 μl variety conc. of Conjugate A or Conjugate B (total 8 conc.) into TB, NSB and blank well
      f. Incubate 2 h on ice
      g. Prepare standards for the 160, 80, 40, and 20 nM concentrations-200 μL of the stock solutions (Tubes 1, 2, 3, 4 in duplicates) to be added to another gamma counting tube
      h. Tap plate to dislodge cells from plate and resuspend cells in each well of the plate
      i. Transfer the binding solution and cells from each well into 1.5 mL tubes
      j. Wash plate with 1 mL cold PBS and transfer to tubes
      k. Spin max speed for 15 sec
      l. Aspirate solution and add 1 mL of cold 1×PBS to tubes
      m. Repeat wash for a total of (2) PBS washes
      n. Centrifuge and aspirate
      o. Lyse cells with 300 μl 1% Triton X-100. Incubate tubes for 30 sec at room temperature with gentle rocking
      p. Transfer 250 μl lysate cells into designated gamma counting tube
      q. Count standards on dose calibrator
      r. Count samples and standards on gamma counter
      s. Determine protein concentration (BCA assay) using the remaining lysate (25 μL)
      t. Calculate Kd using GraphPad Prism software. Analyze the direct binding curve using non-linear curve fitting with a one-site binding model.

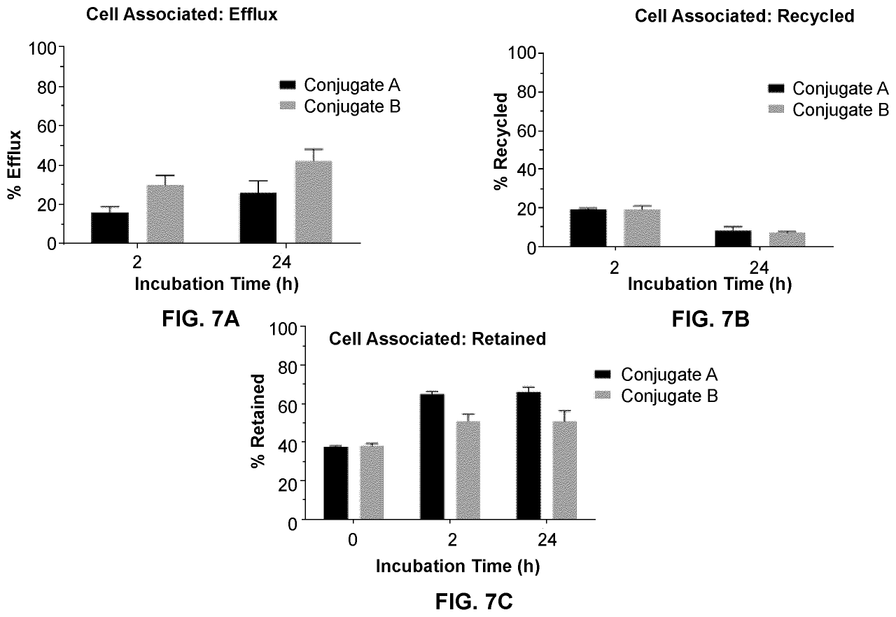

The results from the in vitro binding study are shown in FIGS. 5A and 5B. It was observed that the two EGFRvIII radioimmunoconjugates, i.e., Conjugates A and B, exhibited high binding affinities of 6.78 nM and 2.75 nM, respectively.

### Example 7. Internalization Evaluation of [¹⁷⁷Lu]-Compound C-Anti-EGFRvIII Conjugates

A study was conducted to evaluate the internalization of [¹⁷⁷Lu]-Compound C-anti-EGFRvIII conjugates, i.e., Conjugates A and B, with the U87-EGFRvIII glioblastoma cell line over-expressing EGFRvIII (obtained from National Research Council of Canada) following the protocol described below.

The main purpose of this study is to: 1) quantitatively measure the amount of radiolabeled test article, i.e., EGFRvIII radioimmunoconjugate, on the cell surface and inside cells; and 2) determine the amount of internalized test article that is retrieved in the medium, retained in the cells, or recycled back on the cell surface.

The materials used in this study are summarized in Table 4 below.

TABLE 4
Materials

| | MW or | | | | |
|---|---|---|---|---|---|
| Name | amount | | Company | Catalog No. | Characteristics |
| Test Article (mAb) | 144 | kDa | Fusion | Conjugate A-03 | Monoclonal |
| | | | | Conjugate B-03 | antibody |
| PBS | | | | | pH 7.4 |
| BSA | ~66 | kDa | Sigma | A7906 | pH 6.5-7.5/1% in |
| | | | | | 0.15M NaCl |
| FBS | 500 | ml | Invitrogen | 12483-020 | Qualified, Canada |
| | | | | | origin |
| Trypsin/EDTA | | | Sigma | T4049 | 0.25%, 0.2 g EDTA |
| Trypan blue | 960.81 | | Sigma | T8154 | Sterile-filtered, cell |
| | | | | | culture tested |
| DMEM | 500 | ml | Invitrogen | 11995-073 | With high glucose, |
| | | | | | L-glutamine, Phenol |
| | | | | | Red, Na Pyruvate, |
| | | | | | Without HEPES |

| | | | | |
|---|---|---|---|---|
| Pencillin/streptomycin | | Sigma | P0781 | With 10,000 unit of |
| | | | | Pen and 10 mg |
| | | | | Strep/ml in Saline |
| NaCl | 58.44 | Sigma | S3014 | |
| CaCl₂ | 110.98 | Sigma | C5670 | Anhydrous |
| NaAcetate | 82.03 | Sigma | S8750 | |
| NaOH | 40 | Sigma | S5881 | pellet |
| HCl | 36.46 | Sigma | H1758 | 36.5-38% |
| Triton X-100 | Ave. 625 | Sigma | T8787 | Molecular biology |
| | | | | grade |

### Buffers:

3. Mild acid wash buffer (pH 4.6) was prepared with 150 mM NaCl, 1 mM CaCl₂), and 20 mM NaAc.
4. Acid wash buffer (pH 2.5) was prepared with 500 mM NaCl and 200 mM NaAc.
5. Cell lysate buffer Triton was prepared with 1.0% Triton X-100.

This study followed the procedures below:

1. Cell preparation:
a. 16-24 hours prior to assay start, harvest U87-EGFRvIII cells: Remove medium from cells in a T75, wash with PBS and resuspend the cells in 1 mL of growth medium, pipetting up and down to resuspend. Fill a hemocytometer (10 μL) chamber and count the number of cells present.
b. Resuspend cells in medium to a final concentration of 3×10⁵ cells/ml.
c. Plate 3×10⁵ cells into designated well of the 24-well plates and gently tap to evenly distribute cells.
d. Plate three blank (no cells) wells filled with 1 mL DMEM media/well.
e. Incubate overnight at 37° C. and 5% CO₂.
   Plate 0 h, 2 h, 24 h for each antibody (6 plates):

### 2. Test Article and Standards Preparation:

a. Prepare 15 mL of 10 nM (about 2×Kd) radiolabeled test article from stock solution using medium without FBS (DMEM):
   Conjugate A Stock=0.421 mg/ml=2.8 μM
      15000 uL×10 nM=2.8 μM (x μL)
      53.6 μL of Conjugate A stock antibody into 15 mL DMEM (no FBS)
   Conjugate B Stock=0.448 mg/ml=2.99 μM
      15000 uL×10 nM=2.99 μM (x μL)
      50.2 μL of Conjugate B stock antibody into 15 mL DMEM (no FBS)
b. Prepare 3 standard aliquots of 800 μL each per tube (and read on dose calibrator the next day).

### 3. Procedure:

a. Remove medium from plates.
b. Serum starvation: add 1 mL fresh medium without FBS to each well for 1 hour at 37° C.
c. Wash plates once with 1 mL PBS (sterilized).
d. Load 500 μL Test article into all wells. Prepare test article in medium less than 15 minutes prior to addition.
e. Incubate plates for 2 h at 37° C.
f. After incubation, immediately remove plates from incubator and place on ice.
g. Process plates as follows:
   1) Plate 2 h and 24 h:
      Transfer medium into pre-labelled gamma-counting tubes (non-bound)
      Wash cells once with cold PBS (1 mL) and add into non-bound tube
      Add mild acid wash buffer (pH=4.6) (500 μL) into all wells
      Incubate 15 sec at 4° C. and remove solution into pre-labelled gamma-counting tube (membrane-bound)
      Add 1 mL warmed medium into all wells and incubate plates at 37° C. with 5% CO₂ for designated time (2 h and 24 h)
   2) Plate 0 h:
      Transfer medium into pre-labelled gamma-counting tube (non-bound)
      Wash plates once with 1 ml cold PBS and add to non-bound tube
      Add 500 μL strong acid wash buffer (pH=2.5) into wells
      Incubate 5 sec on ice
      Collect acid wash buffer into pre-labelled gamma-counting tube (membrane-bound)
      Lyse cells with 300 μL 1% Triton X-100 buffer for 30 sec at room temperature with mild shaking
      Transfer 250 μL lysate into designated gamma counting tube (internalized)
      Count samples and standard on gamma counter the next day
h. Post Incubation
   1) Plate 2 h and 24 h:
      Stop incubation at various time by placing the plates on ice
      Transfer medium into pre-labelled gamma-counting tube (efflux)
      Wash plates once with 1 mL cold PBS and add to efflux tube
      Add 500 μL strong acid wash buffer (pH=2.5) into wells
      Incubate 5 sec on ice
      Collect acid wash buffer into pre-labelled gamma-counting tube (recycled)
      Lyse cells with 300 μL 1% Triton X-100 for 30 sec at room temperature with mild shaking
      Transfer 250 μL lysate cells into designated gamma counting tube (retained)
      Count samples and standard on gamma counter the next day

### 4. Analysis

a. Calculate the percentage of non-bound, membrane-bound, internalized, efflux, recycled or retained Test Article accordingly.
   i. Total Activity Calculations:
      Percent free (non-bound; F)=CPM (non-bound)/CPM (non-bound+membrane-bound+internalized)
      Percent cell-associated=100-percent free
      Percent internalized=CPM (internalized)/CPM (non-bound+membrane-bound+internalized)
   ii. Cell-Associated Activity Calculations:
      For 0 h plate (t=0)
         Percent of membrane-bound=CPM (membrane-bound)/CPM (membrane-bound+internalized)
         Percent internalized=CPM (internalized)/CPM (membrane-bound+internalized)
      For 2, 24 h plates
         Percent of membrane-bound (at t=0)=CPM (membrane-bound)/CPM (membrane-bound+efflux+recycled+retained)
         Percent internalized (at t=0)=CPM (efflux+recycled+retained)/CPM (membrane-bound+efflux+recycled+retained)
   iii. Internalized Activity Calculations (t=2 h or 24 h):
      Percent efflux=CPM (efflux)/CPM (efflux+recycled+retained).
      Percent recycled=CPM (recycled)/CPM (efflux+recycled+retained)
      Percent retained=CPM (retained)/CPM (efflux+recycled+retained)
   b. CPM (internalized) and CPM (retained) need to be adjusted for volume (300/250), and the 24 h counts need to be adjusted for decay using the standards.
   c. Plot percent of cell-associated internalized Test Article (ii: t=0) and retained Test Article (iii; t=2 and 24 hours) vs. incubation time using GraphPad Prism®) software.

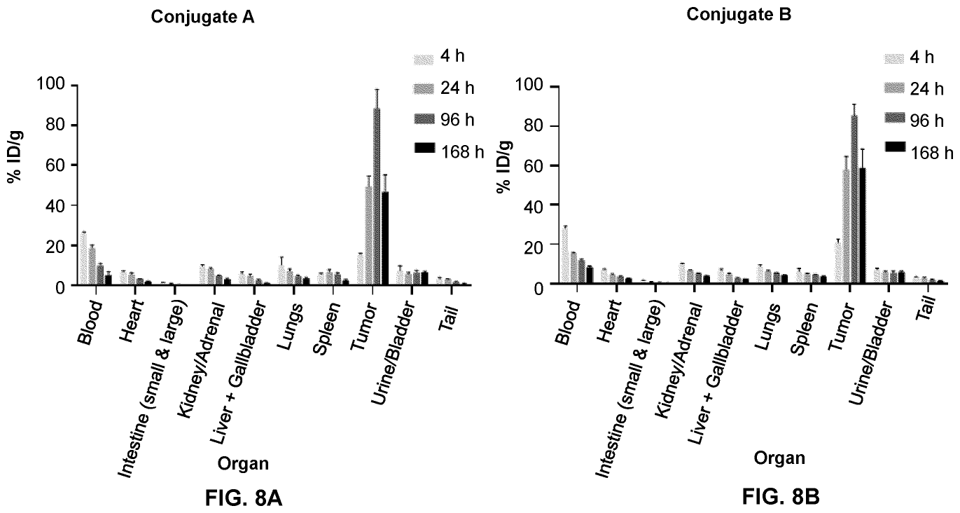

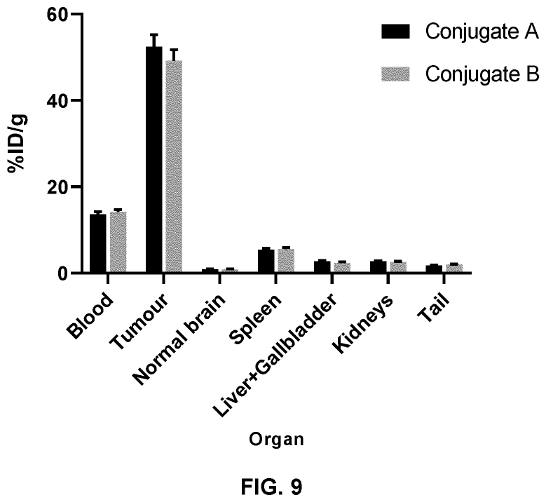

The results from the internalization study are shown in FIGS. 6A-6B and 7A-7C. As shown in FIGS. 6A and 6B, both Conjugates A and B had about 70% membrane-bound and about 30% internalized at 24 hr with no significant difference between the two conjugates.

As shown in FIGS. 7A-7C, both Conjugates A and B showed similar kinetics. Compared with Conjugate A, Conjugate B had slightly higher efflux (42% vs 26% at 24 hour). % Retained was slightly lower in Conjugate B (51%) as compared to Conjugate A (66%) at 24 hour which may be attributed to the higher efflux with Conjugate B.

### Example 8. In Vivo Biodistribution of [¹⁷⁷Lu]-Compound C-Anti-EGFRvIII Conjugates in Subcutaneous U87-EGFRvIII Model

A U87-EGFRvIII cell line xenograft mouse model was used to assess the in vivo biodistribution of [¹⁷⁷Lu]-DOTA-anti-EGFRvIII conjugates. Two [¹⁷⁷Lu]-DOTA-anti-EGFRvIII conjugates, i.e., Conjugates A and B, were synthesized using a pure R enantiomer of Compound C (see Example 4), two humanized variants of anti-EGFRvIII antibody 4E11, and lutetium-177.

Groups of tumor-bearing animals were injected intravenously with [¹⁷⁷Lu]-DOTA-anti-EGFRvIII conjugates. Doses contained about 9.6-9.8 microcuries (μCi)/μg of activity on 2 μg (0.1 mg/kg) of antibody. Animals were euthanized at 4 h, 24 h, 96 h, and 168 h after injection to determine levels of radioactivity in the blood, heart, intestines, kidneys, liver, lungs, spleen, tumor, urine, and tail (n=3 per time point).

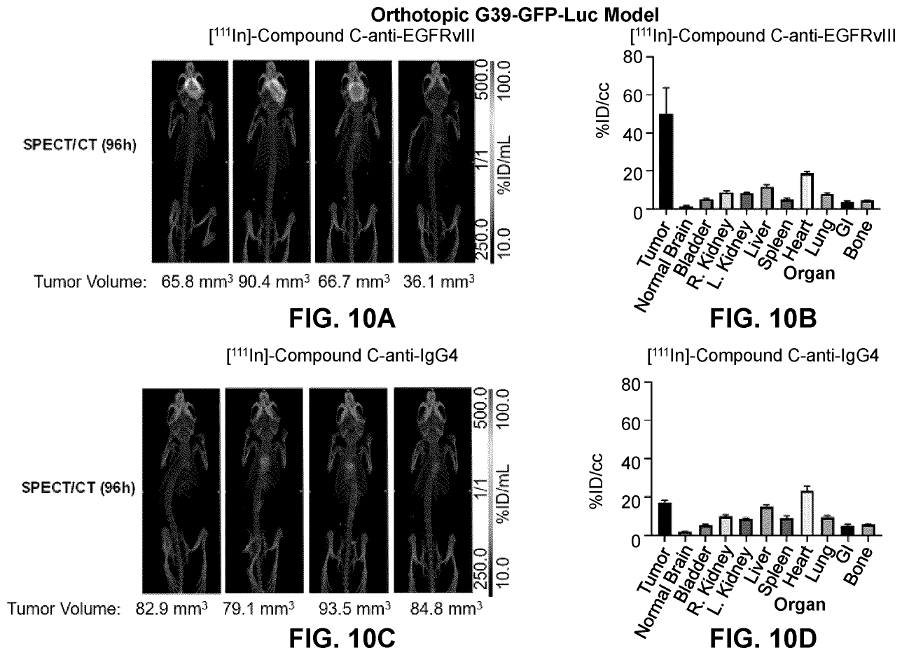

Results were expressed as the percentage injected dose per gram of tissue (% ID/g) and are depicted in FIGS. 8A and 8B (for Conjugate A and Conjugate B, respectively). Both conjugates showed clearance of radioactivity from the blood over 168 h, low uptake of [¹⁷⁷Lu]-DOTA-anti-EGFRvIII in normal tissues, and high uptake of [¹⁷⁷Lu]-DOTA-anti-EGFRvIII in tumors. Superior tumor uptake at 96 h was demonstrated for both conjugates. For example, Conjugates A and B showed excellent tumor uptake with about 88.6±9.5% ID/g and about 85.5±9.9% ID/g, respectively, at 96 h after dosing in the subcutaneous U87-EGFRvIII model.

### Example 9. In Vivo Biodistribution of [¹⁷⁷Lu]-Compound C-Anti-EGFRvIII Conjugates in a U87-EGFRvIII-GFP-Luc Orthotopic Model

A U87-EGFRvIII-GFP-Luc orthotopic model was used to assess the in vivo biodistribution of radiolabeled anti-EGFRvIII conjugates, i.e., Conjugates A and B.

Groups of tumor-bearing animals were injected intravenously with [¹⁷⁷Lu]-DOTA-anti-EGFRvIII. Doses contained about 9.6-9.8 microcuries (μCi)/μg of activity on 2 μg (0.1 mg/kg) of antibody. Animals were euthanized at 96 h after injection to determine levels of radioactivity in the blood, tumor, normal brain, spleen, liver, kidneys, and tail (n=10 per time point for Conjugate A and n=10 per time point for Conjugate B).

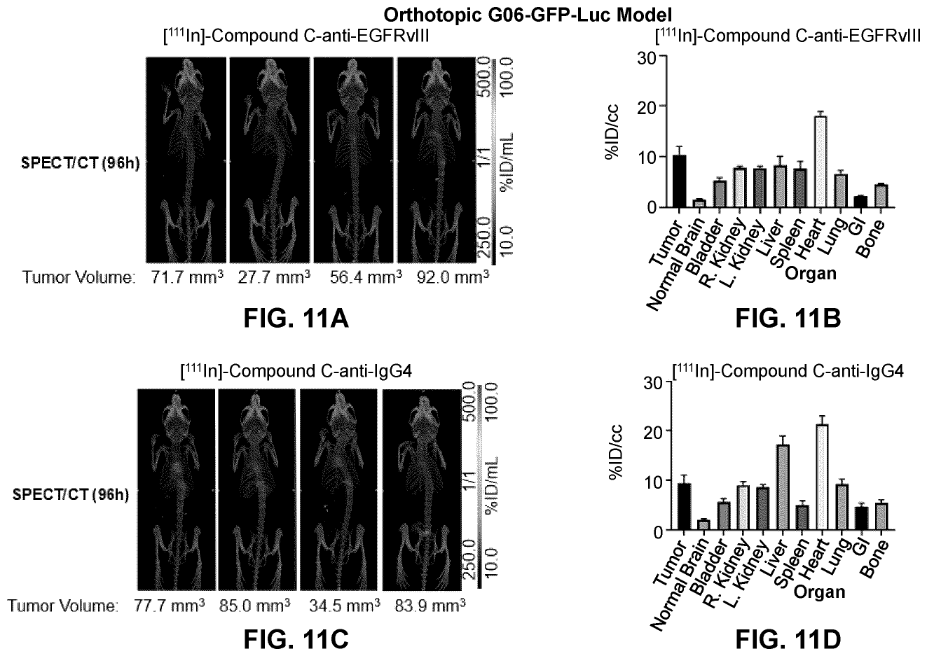

Results were expressed as the percentage injected dose per gram of tissue (% ID/g) and are depicted in FIG. 9. Both conjugates showed good blood uptake of [¹⁷⁷Lu]-DOTA-anti-EGFRvIII, low uptake of [¹⁷⁷Lu]-DOTA-anti-EGFRvIII in normal tissues, and high uptake of [¹⁷⁷Lu]-DOTA-anti-EGFRvIII in brain tumors at 96 h. For example, Conjugates A and B showed excellent brain tumor uptake with about 52.4±2.9% ID/g and about 49.1±2.6% ID/g, respectively, at 96 h after dosing in the U87-EGFRvIII-GFP-Luc orthotopic model. High brain tumor uptake indicates good blood-brain barrier (BBB) penetrance and is likely due to leaky vasculature.

### Example 10. In Vivo Biodistribution of [¹¹¹In]-Compound C-Anti-EGFRvIII and [¹¹¹In]-Compound C-IgG4 in Orthotopic Patient-Derived Xenograft (PDX) Models

Target specificity and tumor uptake of [¹¹¹In]-Compound C-anti-EGFRvIII (see Example 5 above) was investigated in an imaging biodistribution study using orthotopic glioblastoma multiforme (GBM) PDX models with varying degrees of blood-brain barrier (BBB) permeability. Although all GBM tumors have some degree of BBB permeability as a characteristic of the disease, this can vary considerably between tumors and even within the same tumor. The large size of antibodies (about 150 kDa) prevents them from crossing an intact BBB, and intravenous administration of radiolabeled anti-EGFRvIII immunoconjugates may be dependent on BBB permeability for delivery to GBM tumors. To characterize the extent of tumor uptake after intravenous administration of [¹¹¹In]-Compound C-anti-EGFRvIII, an imaging biodistribution study was performed in a GBM PDX model with a relatively intact BBB (G06-GFP-Luc) and another model with a completely disrupted BBB (G39-GFP-Luc). In addition, to validate the target specificity, the biodistribution of a non-targeted [¹¹¹In]-Compound C-IgG4 (see Example 5 above) was also assessed for comparison.

Orthotopic GBM PDX tumors were established in 7-8 week old female Balb/c athymic nude mice by intracranial injection of 3×10⁵ G06-GFP-Luc or 2.5×10⁵ G39-GFP-Luc cells in 3 μL PBS. Cells were injected into the right cerebral hemisphere 1 mm posterior from bregma, 1.5 mm to the right of the sagittal suture, and at a depth of 2.5 mm. Bioluminescence 3D tomography (BLT) was performed 13 days after intracranial injections to confirm the presence of tumors and determine tumor volumes. 150 mg/kg D-luciferin in PBS was injected intraperitoneally into mice 15 min prior to imaging. Mice were anesthetized with isoflurane and placed on the imaging bed. Images were acquired using the optical imaging (OI) module of the VECTor⁶CT SPECT/PET/CT/OI imaging system and reconstructed using MiLabs BLT Recon 1.0 software. Tumor volume was quantified using Imalytics software and region of interest (ROI) analysis. Mice were triaged into groups based on tumor size such that each group had a relatively equal distribution of tumor volumes.

Tumor-bearing mice were injected intravenously via the lateral tail vein with 0.2 mL of [¹¹¹In]-Compound C-anti-EGFRvIII or [¹¹¹In]-Compound C-IgG4 containing 100 μCi of radioactivity (10.8-11.8 μg of antibody) formulated in 20 mM sodium citrate pH 5.5, 0.82% NaCl, 0.01% Tween 80 (n=4 mice/compound). BLT and SPECT/CT imaging was performed ⁹⁶ hours post-injection using the VECTor⁶CT SPECT/PET/CT/OI imaging system to determine the tissue biodistribution. Images were reconstructed using MiLabs BLT Recon 1.0 software and tumor/normal tissue uptake was determined using PMOD software. Uptake was calculated as percentage of injected dose per cubic centimeter of tissue (% ID/cc).

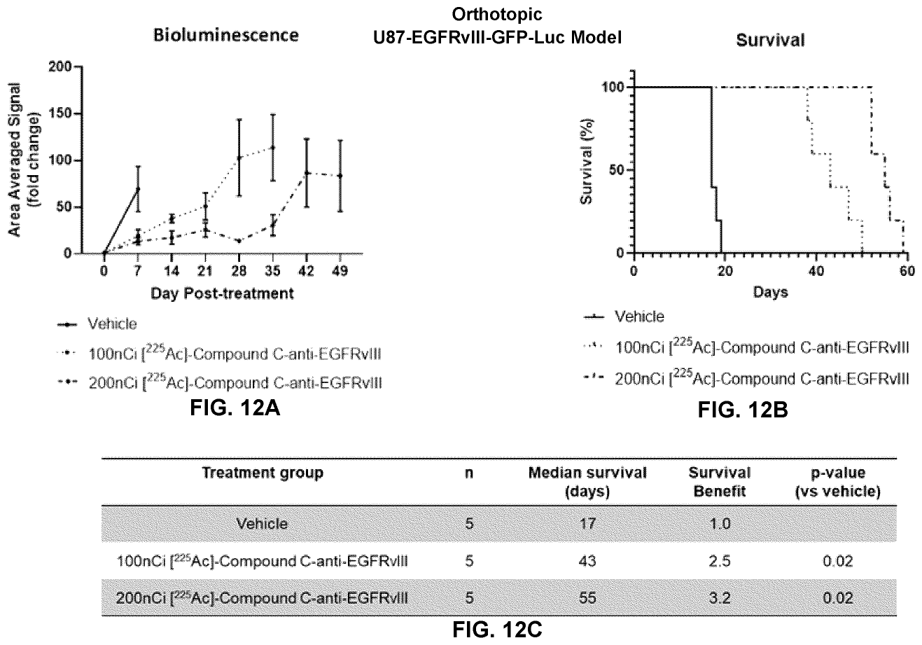

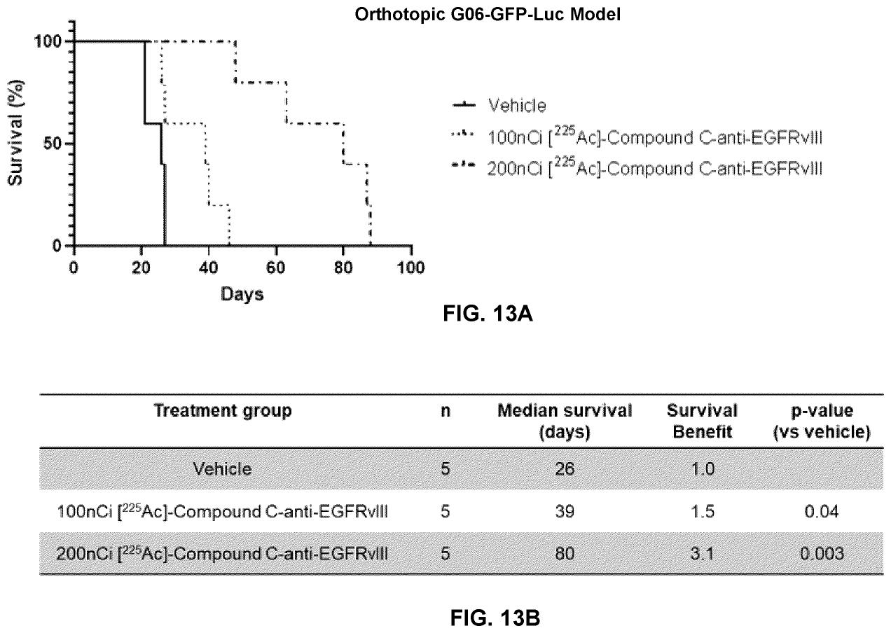

In the G39-GFP-Luc PDX model with a disrupted BBB, [¹¹¹In]-Compound C-anti-EGFRvIII biodistribution data demonstrated high tumor uptake of 50.2±13.6% ID/cc at 96 h. In comparison, non-targeting [¹¹¹In]-Compound C-IgG4 had a tumor uptake of 17.4±1.2% ID/cc at 96 h, confirming that the majority of [¹¹¹In]-Compound C-anti-EGFRvIII tumor uptake is target specific. Importantly, normal brain showed very low uptake (1.6-2.1% ID/cc) providing further evidence of tumor-specific uptake of [¹¹¹In]-Compound C-anti-EGFRVIII (FIGS. 10B and 11D). All other normal organs evaluated had an uptake ≤10% ID/cc with the exception of highly perfused organs such as liver and heart, which represents the majority of the blood pool.

In the G06-GFP-Luc PDX model with a relatively intact BBB, [¹¹¹In]-Compound C-anti-EGFRvIII biodistribution data demonstrated a tumor uptake of 10.4±1.6% ID/cc at 96 h while non-targeting [¹¹¹In]-Compound C-IgG4 had a tumor uptake of 9.4±1.7% ID/cc at 96 h (FIGS. 11B and 11D). These results are consistent with the inability of antibodies to cross the intact BBB in this model. Normal tissue uptake was similar to that of the tumor (<10% ID/cc) with the exception of highly perfused organs such as liver and heart, which represents the majority of the blood pool.

The results of the imaging biodistribution studies confirm that uptake of [¹¹¹In]-Compound C-anti-EGFRvIII is highly tumor-specific with minimal off-target uptake in normal tissues. It was further demonstrated that high tumor uptake of [¹¹¹In]-Compound C-anti-EGFRvIII can be achieved via intravenous administration in tumors with a highly disrupted BBB, but uptake is restricted by an intact BBB.

### Example 11. Single-Dose Radiotherapeutic Efficacy of [²²⁵Ac]-Compound C-Anti-EGFRvIII in Orthotopic Glioblastoma Mouse Models

The single-dose radiotherapeutic efficacy of [²²⁵Ac]-Compound C-anti-EGFRvIII (see Example 5 above) was determined using orthotopic glioblastoma multiforme (GBM) models generated with the cell line U87-EGFRvIII-GFP-Luc or the patient-derived xenograft (PDX) model, G06-GFP-Luc. Both the GBM cell line and PDX cells were transduced with lentiviral particles expressing green fluorescent protein (GFP) and luciferase (Luc) for in vivo and ex vivo imaging purposes. GBM brain tumors were established in 7-8 week old female Balb/c athymic nude mice by intracranial injection of 2×10⁴ U87-EGFRvIII-GFP-Luc cells or 3×10⁵ G06-GFP-Luc in 3 μL PBS. Cells were injected into the right cerebral hemisphere 1 mm posterior from bregma, 1.5 mm to the right of the sagittal suture, and at a depth of 2.5 mm. Bioluminescence imaging (BLI) was performed 8-10 days after intracranial injections to confirm the presence of tumors and quantify tumor burden. 150 mg/kg D-luciferin in PBS was injected intraperitoneally into mice 15 min prior to imaging. BLI signal was quantified in a region of interest (ROI) drawn around the tumor and expressed as area averaged signal. The area averaged signal represents a measurement of tumor burden and mice were triaged into groups such that the average BLI tumor signal was equal across groups.

Therapy was initiated 10-13 days post-intracranial injection and after tumors were verified by imaging. Tumor-bearing mice were injected intravenously (IV) via the lateral tail vein with 0.2 mL [²²⁵Ac]-Compound C-anti-EGFRvIII formulated in 20 mM sodium citrate pH5.5, 0.82% NaCl, 0.01% Tween 80 or vehicle alone for control mice (n=5/group). Actinium-225 radiolabeled test articles contained 100-200 nCi of activity (up to 4 μg of antibody protein/dose). Mice were weighed three times per week and monitored daily for signs of brain tumor development such as hunched posture, lethargy, seizures, impaired mobility, paralysis, or enlarged skull. For the U87-EGFRVIII-GFP-Luc cell line model, BLI was used to monitor tumor burden and response to therapy over time. Mice were imaged once per week as described above, and BLI signals were plotted as a fold change from the pre-treatment area averaged tumor signal to determine the therapeutic response. Animals were euthanized when humane endpoints were reached (>20% weight loss, BC2) or signs of brain tumors developed (as described above). Overall survival was plotted on a Kaplan-Meier survival curve. The log rank (Mantel-Cox) test was used to statistically compare the survival between groups and determine p-values. To facilitate the interpretation of treatment response between models, the survival benefit for a given treatment was calculated by determining the ratio of the median survival for mice receiving the treatment versus vehicle control.

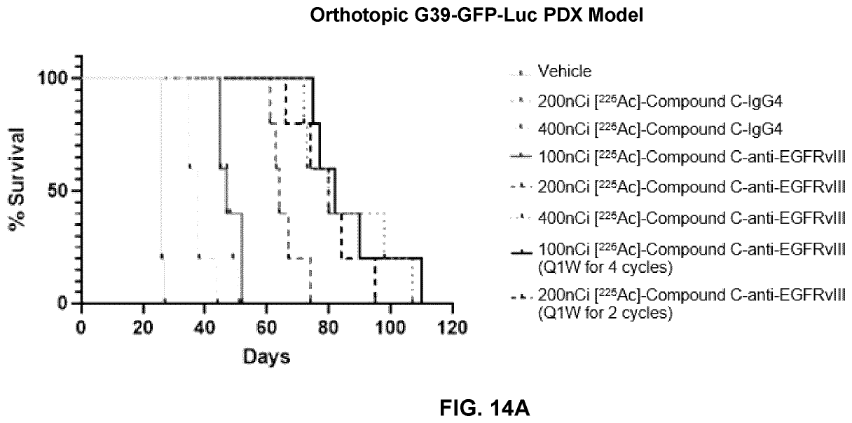

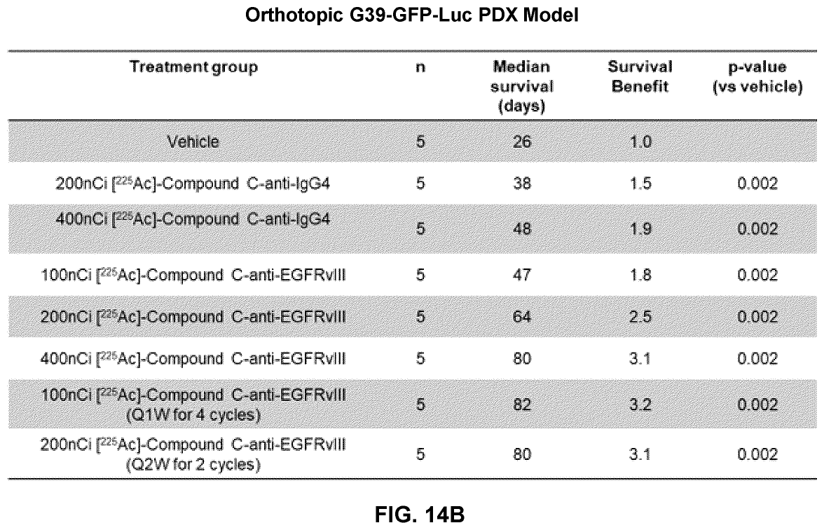

Radiotherapeutic studies of [²²⁵Ac]-Compound C-anti-EGFRvIII in the U87-EGFRvIII-GFP-Luc cell line model demonstrated significant therapeutic efficacy at both 100 nCi and 200 nCi doses. BLI analysis revealed that tumor growth was significantly suppressed in comparison to the vehicle control group (FIG. 12A). In the 200 nCi dose group, tumors showed near complete tumor suppression up until day 28. The suppression of tumor growth in response to treatment resulted in a significant survival benefit. The median survival of vehicle control was 17 days versus 43 days in the 100 nCi group and 55 days in the 200 nCi group which translates to a survival benefit of 2.5 and 3.2, respectively. These differences in survival were statistically significant (p<0.05, Mantel-Cox test) (FIG. 12B; FIG. 12C). Similarly, the G06-GFP-Luc PDX model showed a significant survival benefit in response to treatment with [²²⁵Ac]-Compound C-anti-EGFRvIII. The median survival of the vehicle control group was 26 days while the 100 nCi and 200 nCi dose groups had a median survival of 39 days and 80 days, respectively. In comparison to the vehicle controls, the 200 nCi dose group showed a significant survival benefit of 3.1, while the 100 nCi group showed a more modest survival benefit of 1.5 (FIGS. 13A and 13B). Collectively, these results demonstrate that single-dose [²²⁵Ac]-Compound C-anti-EGFRvIII significantly slows tumor growth in a dose-dependent manner and extends survival in multiple orthotopic GBM xenograft models.

### Example 12. Fractionated Multi-Dose Radiotherapeutic Efficacy of [²²⁵Ac]-Compound C-Anti-EGFRvIII in Orthotopic Glioblastoma Mouse Models

The radiotherapeutic efficacy of [²²⁵Ac]-Compound C-anti-EGFRVIII was examined using an orthotopic GBM patient-derived xenograft (PDX) model. Multiple fractionated doses of [²²⁵Ac]-Compound C-anti-EGFRvIII or [²²⁵Ac]-Compound C-IgG4 (see Example 5 above) were compared to single dose administration to determine the optimal dosing regimen to maximize therapeutic efficacy. Aside from the dosing schedule, the study was carried out as described above for the single dose radiotherapeutic efficacy studies.

Mice bearing orthotopic G39-GFP-Luc tumors were administered [²²⁵Ac]-Compound C-anti-EGFRvIII at a single dose of 100, 200, or 400 nCi or a total cumulative radiochemical dose of 400 nCi fractionated as follows: 100 nCi dosed once weekly for 4 weeks (100 nCi×4) or 200 nCi dosed every 2 weeks for 2 doses (200 nCi×2). Each dose contained 8.6 μg of antibody protein. Control groups were administered a single dose of [²²⁵Ac]-Compound C-IgG4 containing either 200 nCi or 400 nCi of radioactivity (9.4 μg antibody protein/dose) or vehicle alone (n=5 mice/group).

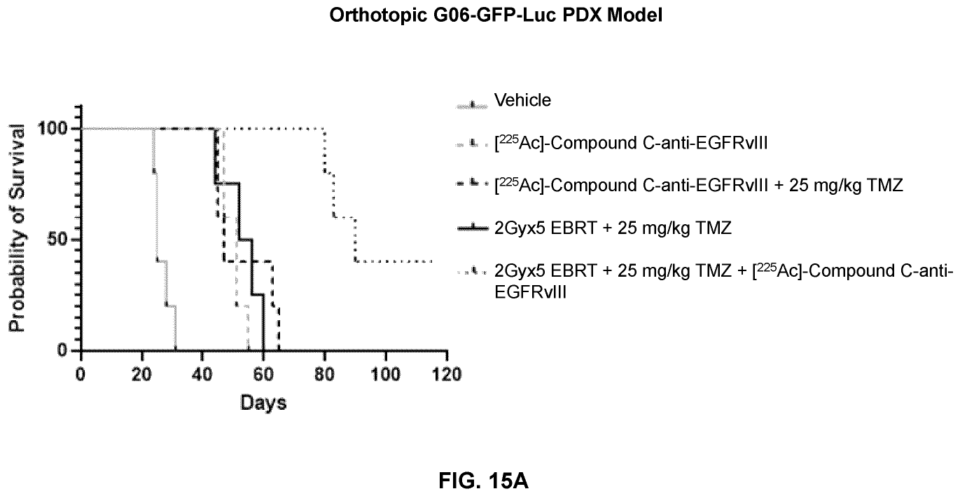

Survival analysis showed a dose-dependent increase in survival in response to treatment with [²²⁵Ac]-Compound C-anti-EGFRvIII. The survival benefits of the 400 nCi [²²⁵Ac]-Compound C-anti-EGFRvIII single-dose or fractionated dose groups were comparable with survival benefit ratios of 3.1, 3.2, and 3.1 for the 400 nCi single dose, fractionated 200 nCi×2, and fractionated 100 nCi×4 dose groups, respectively. These results further demonstrated that efficacy was related to the total radioactivity dose, regardless of the dosing schedule. In addition, the survival benefit of 400 nCi single-dose [²²⁵Ac]-Compound C-IgG4 was significantly lower (1.9), confirming that at least some of the therapeutic efficacy of [²²⁵Ac]-Compound C-anti-EGFRvIII is target-mediated (FIGS. 14A and 14B).

### Example 13. Therapeutic Efficacy of [²²⁵Ac]-Compound C-Anti-EGFRvIII in Combination with Standard of Care

The standard of care (SoC) for GBM patients is maximal surgical resection followed by external beam radiation therapy (EBRT) in combination with the chemotherapeutic temozolomide (TMZ). The therapeutic efficacy of combined SoC and [²²⁵Ac]-Compound C-anti-EGFRvIII in comparison to single-agent was tested using the orthotopic G06-GFP-Luc GBM PDX model. Tumors were engrafted and verified by BLT imaging as described above. Tumor bearing mice were treated with a single intravenous dose (0.2 mL) of vehicle alone, 200 nCi dose [²²⁵Ac]-Compound C-anti-EGFRvIII, or 200 nCi dose [²²⁵Ac]-Compound C-anti-EGFRvIII in combination with TMZ (25 mg/kg administered daily for 5 days via oral gavage). Two additional treatment groups (SoC alone and combination group) received 2 Gy EBRT once daily for 5 consecutive days in combination with 25 mg/kg temozolomide (administered daily for 5 days via oral gavage 1 hr prior to EBRT). The combination group then subsequently received a single dose of 200 nCi [²²⁵Ac]-Compound C-anti-EGFRvIII 6 days after completion of EBRT/TMZ treatments (n=5 mice/group).

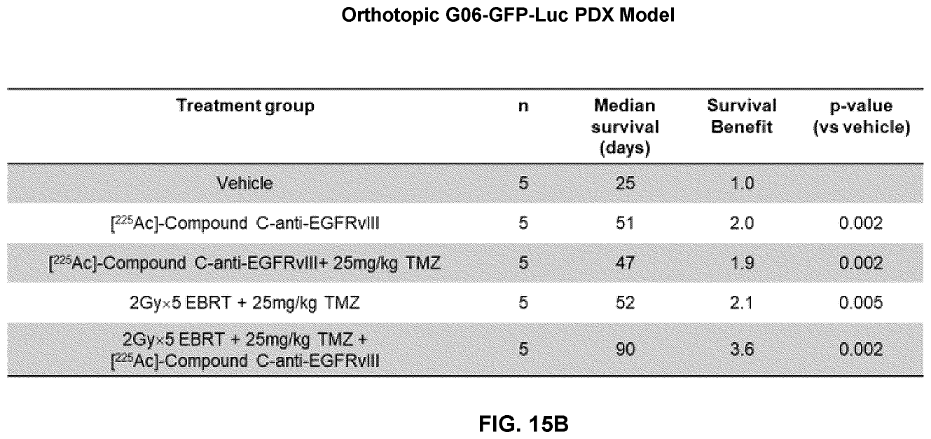

Survival analysis demonstrated that SoC alone (2 Gyx5 EBRT+25 mg/kg TMZ), 200 nCi [²²⁵Ac]-Compound C-anti-EGFRvIII, and 200 nCi [²²⁵Ac]-Compound C-anti-EGFRvIII in combination with TMZ all significantly extend survival in comparison to vehicle controls with a survival benefit of approximately 2 for all. However, combined treatment with SoC (2 Gyx5 EBRT+25 mg/kg TMZ) followed by 200 nCi [²²⁵Ac]-Compound C-anti-EGFRvIII resulted in a significant extension in survival (survival benefit of 3.6) compared to SoC alone, [²²⁵Ac]-Compound C-anti-EGFRvIII, or [²²⁵Ac]-Compound C-anti-EGFRvIII in combination with TMZ (survival benefit of approximately 2) (FIGS. 15A and 15B). These results suggest that combined treatment of [²²⁵Ac]-Compound C-anti-EGFRvIII and SoC may offer a significant survival benefit in comparison to conventional SoC alone treatment for GBM.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

TABLE 5
Amino Acid Sequences.
In some sequences, CDRs are underlined and indicated in bold

| Seq. | | |
|---|---|---|
| ID | Description | Sequence |
| 1 | Wild type human | MRPSGTAGAALLALLAALCPASRALEEKKVCQGTSNKLTQLGTFEDH |
| | EGFR amino | FLSLQRMFNNCEVVLGNLEITYVQRNYDLSFLKTIQEVAGYVLIALNTV |
| | acid sequence | ERIPLENLQIIRGNMYYENSYALA VLSNYDANKTGLKELPMRNLQEILH |
| | (signal peptide in | GAVRFSNNPALCNVESIQWRDIVSSDFLSNMSMDFQNHLGSCQKCDPS |
| | bold and | CPNGSCWGAGEENCQKLTKIICAQQCSGRCRGKSPSDCCHNQCAAGCT |
| | underlined) | GPRESDCLVCRKFRDEATCKDTCPPLMLYNPTTYQMDVNPEGKYSFGA |
| | | TCVKKCPRNYVVTDHGSCVRACGADSYEMEEDGVRKCKKCEGPCRK |
| | | VCNGIGIGEFKDSLSINATNIKHFKNCTSISGDLHILPVAFRGDSFTHTPP |
| | | LDPQELDILKTVKEITGFLLIQAWPENRTDLHAFENLEIIRGRTKQHGQF |
| | | SLAVVSLNITSLGLRSLKEISDGDVIISGNKNLCYANTINWKKLFGTSGQ |
| | | KTKIISNRGENSCKATGQVCHALCSPEGCWGPEPRDCVSCRNVSRGRE |
| | | CVDKCNLLEGEPREFVENSECIQCHPECLPQAMNITCTGRGPDNCIQCA |
| | | HYIDGPHCVKTCPAGVMGENNTLVWKYADAGHVCHLCHPNCTYGCT |
| | | GPGLEGCPTNGPKIPSIATGMVGALLLLLVVALGIGLFMRRRHIVRKRT |
| | | LRRLLQERELVEPLTPSGEAPNQALLRILKETEFKKIKVLGSGAFGTVYK |
| | | GLWIPEGEKVKIPVAIKELREATSPKANKEILDEAYVMASVDNPHVCRL |
| | | LGICLTSTVQLITQLMPFGCLLDYVREHKDNIGSQYLLNWCVQIAKGM |
| | | NYLEDRRLVHRDLAARNVLVKTPQHVKITDFGLAKLLGAEEKEYHAE |
| | | GGKVPIKWMALESILHRIYTHQSDVWSYGVTVWELMTFGSKPYDGIPA |
| | | SEISSILEKGERLPQPPICTIDVYMIMVKCWMIDADSRPKFRELIIEFSKM |
| | | ARDPQRYLVIQGDERMHLPSPTDSNFYRALMDEEDMDDVVDADEYLIP |
| | | QQGFFSSPSTSRTPLLSSLSATSNNSTVACIDRNGLQSCPIKEDSFLQRYS |
| | | SDPTGALTEDSIDDTFLPVPEYINQSVPKRPAGSVQNPVYHNQPLNPAPS |
| | | RDPHYQDPHSTAVGNPEYLNTVQPTCVNSTFDSPAHWAQKGSHQISLD |
| | | NPDYQQDFFPKEAKPNGIFKGSTAENAEYLRVAPQSSEFIGA |
| 2 | Wild type human | CTGGAGGAAAAGAAAGTTTGCCAAGGCACGAGTAACAAGCTCACGC |
| | EGFR | AGTTGGGCACTTTTGAAGATCATTTTCTCAGCCTCCAGAGGATGTTC |
| | ectodomain | AATAACTGTGAGGTGGTCCTTGGGAATTTGGAAATTACCTATGTGCA |
| | cDNA sequence | GAGGAATTATGATCTTTCCTTCTTAAAGACCATCCAGGAGGTGGCTG |
| | | GTTATGTCCTCATTGCCCTCAACACAGTGGAGCGAATTCCTTTGGAA |
| | | AACCTGCAGATCATCAGAGGAAATATGTACTACGAAAATTCCTATG |
| | | CCTTAGCAGTCTTATCTAACTATGATGCAAATAAAACCGGACTGAA |
| | | GGAGCTGCCCATGAGAAATTTACAGGAAATCCTGCATGGCGCCGTG |
| | | CGGTTCAGCAACAACCCTGCCCTGTGCAACGTGGAGAGCATCCAGT |
| | | GGCGGGACATAGTCAGCAGTGACTTTCTCAGCAACATGTCGATGGA |
| | | CTTCCAGAACCACCTGGGCAGCTGCCAAAAGTGTGATCCAAGCTGT |
| | | CCCAATGGGAGCTGCTGGGGTGCAGGAGAGGAGAACTGCCAGAAA |
| | | CTGACCAAAATCATCTGTGCCCAGCAGTGCTCCGGGCGCTGCCGTG |
| | | GCAAGTCCCCCAGTGACTGCTGCCACAACCAGTGTGCTGCAGGCTG |
| | | CACAGGCCCCCGGGAGAGCGACTGCCTGGTCTGCCGCAAATTCCGA |
| | | GACGAAGCCACGTGCAAGGACACCTGCCCCCCACTCATGCTCTACA |
| | | ACCCCACCACGTACCAGATGGATGTGAACCCCGAGGGCAAATACAG |
| | | CTTTGGTGCCACCTGCGTGAAGAAGTGTCCCCGTAATTATGTGGTGA |
| | | CAGATCACGGCTCGTGCGTCCGAGCCTGTGGGGCCGACAGCTATGA |
| | | GATGGAGGAAGACGGCGTCCGCAAGTGTAAGAAGTGCGAAGGGCC |
| | | TTGCCGCAAAGTGTGTAACGGAATAGGTATTGGTGAATTTAAAGAC |
| | | TCACTCTCCATAAATGCTACGAATATTAAACACTTCAAAAACTGCAC |
| | | CTCCATCAGTGGCGATCTCCACATCCTGCCGGTGGCATTTAGGGGTG |
| | | ACTCCTTCACACATACTCCTCCTCTGGATCCACAGGAACTGGATATT |
| | | CTGAAAACCGTAAAGGAAATCACAGGGTTTTTGCTGATTCAGGCTT |
| | | GGCCTGAAAACAGGACGGACCTCCATGCCTTTGAGAACCTAGAAAT |
| | | CATACGCGGCAGGACCAAGCAACATGGTCAGTTTTCTCTTGCAGTCG |
| | | TCAGCCTGAACATAACATCCTTGGGATTACGCTCCCTCAAGGAGATA |
| | | AGTGATGGAGATGTGATAATTTCAGGAAACAAAAATTTGTGCTATG |
| | | CAAATACAATAAACTGGAAAAAACTGTTTGGGACCTCCGGTCAGAA |
| | | AACCAAAATTATAAGCAACAGAGGTGAAAACAGCTGCAAGGCCAC |
| | | AGGCCAGGTCTGCCATGCCTTGTGCTCCCCCGAGGGCTGCTGGGGCC |
| | | CGGAGCCCAGGGACTGCGTCTCTTGCCGGAATGTCAGCCGAGGCAG |
| | | GGAATGCGTGGACAAGTGCAACCTTCTGGAGGGTGAGCCAAGGGAG |
| | | TTTGTGGAGAACTCTGAGTGCATACAGTGCCACCCAGAGTGCCTGCC |
| | | TCAGGCCATGAACATCACCTGCACAGGACGGGGACCAGACAACTGT |
| | | ATCCAGTGTGCCCACTACATTGACGGCCCCCACTGCGTCAAGACCTG |
| | | CCCGGCAGGAGTCATGGGAGAAAACAACACCCTGGTCTGGAAGTAC |
| | | GCAGACGCCGGCCATGTGTGCCACCTGTGCCATCCAAACTGCACCT |
| | | ACGGATGCACTGGGCCAGGTCTTGAAGGCTGTCCAACGAATGGGCC |
| | | TAAGATCCCGTCCATCGCC |
| 3 | Human EGFR | LEEKKVCQGTSNKLTQLGTFEDHFLSLQRMFNNCEVVLGNLEITYVQR |
| | Ectodomain | NYDLSFLKTIQEVAGYVLIALNTVERIPLENLQIIRGNMYYENSYALAVL |
| | amino acid | SNYDANKTGLKELPMRNLQEILHGAVRFSNNPALCNVESIQWRDIVSS |
| | sequence | DFLSNMSMDFQNHLGSCQKCDPSCPNGSCWGAGEENCQKLTKIICAQQ |
| | | CSGRCRGKSPSDCCHNQCAAGCTGPRESDCLVCRKFRDEATCKDTCPP |
| | | LMLYNPTTYQMDVNPEGKYSFGATCVKKCPRNYVVTDHGSCVRACG |
| | | ADSYEMEEDGVRKCKKCEGPCRKVCNGIGIGEFKDSLSINATNIKHFKN |
| | | CTSISGDLHILPVAFRGDSFTHTPPLDPQELDILKTVKEITGFLLIQAWPE |
| | | NRTDLHAFENLEIIRGRTKQHGQFSLAVVSLNITSLGLRSLKEISDGDVII |
| | | SGNKNLCYANTINWKKLFGTSGQKTKIISNRGENSCKATGQVCHALCS |
| | | PEGCWGPEPRDCVSCRNVSRGRECVDKCNLLEGEPREFVENSECIQCHP |
| | | ECLPQAMNITCTGRGPDNCIQCAHYIDGPHCVKTCPAGVMGENNTLV |
| | | WKYADAGHVCHLCHPNCTYGCTGPGLEGCPTNGPKIPS |
| 4 | Human | CTGGAAGAGAAGAAAGGCAACTACGTCGTGACCGACCACGGCAGCT |
| | EGFRvIII | GTGTGCGGGCTTGTGGCGCCGATAGCTACGAGATGGAAGAGGACGG |
| | ectodomain | CGTGCGGAAGTGCAAGAAGTGCGAGGGCCCCTGCCGGAAAGTGTGC |
| | cDNA sequence | AACGGCATCGGCATCGGAGAGTTCAAGGACAGCCTGAGCATCAACG |
| | (nucleotides 1- | CCACCAACATCAAGCACTTCAAGAACTGCACCAGCATCAGCGGCGA |
| | 996) | CCTGCACATCCTGCCCGTGGCCTTTAGAGGCGACAGCTTCACCCACA |
| | | CCCCCCCACTGGACCCCCAGGAACTGGACATCCTGAAAACCGTGAA |
| | | AGAGATCACCGGCTTTCTGCTGATTCAGGCCTGGCCCGAGAACCGG |
| | | ACAGACCTGCACGCCTTCGAGAACCTGGAAATCATCCGGGGCAGGA |
| | | CCAAGCAGCACGGCCAGTTTTCTCTGGCCGTGGTGTCCCTGAACATC |
| | | ACCAGCCTGGGCCTGCGGAGCCTGAAAGAAATCAGCGACGGCGACG |
| | | TGATCATCTCCGGCAACAAGAACCTGTGCTACGCCAACACCATCAA |
| | | CTGGAAGAAGCTGTTCGGCACCTCCGGCCAGAAAACAAAGATCATC |
| | | AGCAACCGGGGCGAGAACAGCTGCAAGGCCACAGGACAAGTGTGC |
| | | CACGCCCTGTGTAGCCCTGAGGGCTGTTGGGGACCCGAGCCCAGAG |
| | | ATTGCGTGTCCTGCAGAAACGTGTCCCGGGGCAGAGAATGCGTGGA |
| | | CAAGTGCAACCTGCTGGAAGGCGAGCCCCGCGAGTTCGTGGAAAAC |
| | | AGCGAGTGCATCCAGTGCCACCCCGAGTGTCTGCCCCAGGCCATGA |
| | | ACATTACCTGCACCGGCAGAGGCCCCGACAACTGTATCCAGTGCGC |
| | | CCACTACATCGACGGCCCCCACTGCGTGAAAACCTGTCCTGCTGGCG |
| | | TGATGGGAGAGAACAACACCCTCGTGTGGAAGTACGCCGACGCCGG |
| | | CCATGTGTGCCACCTGTGTCACCCCAAT |
| 5 | Human | LEEKKGNYVVTDHGSCVRACGADSYEMEEDGVRKCKKCEGPCRKVC |
| | EGFRvIII | NGIGIGEFKDSLSINATNIKHFKNCTSISGDLHILPVAFRGDSFTHTPPLDP |
| | ectodomain | QELDILKTVKEITGFLLIQAWPENRTDLHAFENLEIIRGRTKQHGQFSLA |
| | amino acid | VVSLNITSLGLRSLKEISDGDVIISGNKNLCYANTINWKKLFGTSGQKTK |
| | sequence (amino | IISNRGENSCKATGQVCHALCSPEGCWGPEPRDCVSCRNVSRGRECVD |
| | acids 1-332) | KCNLLEGEPREFVENSECIQCHPECLPQAMNITCTGRGPDNCIQCAHYID |
| | | GPHCVKTCPAGVMGENNTLVWKYADAGHVCHLCHPN |
| 6 | Human | SCVRACGADSYEMEEDGVRKCKK |
| | EGFRvIII amino | |
| | acid residues 15 | |
| | to 37 | |
| 7 | 5G6 light chain | DVVMTQTPLTLSVTIGQPASISCKSSQSLLDSDGKTYLNWLLQRPGQSP |
| | variable region | KRLIYLASKLDSGVPDRFTGSGSGTDFTLKISRVEAEDLGVYYCWQAT |
| | (CDRs in bold) | HFPWTFGGGTKLEIK |
| 8 | 5G6 CDRL 1 | KSSQSLLDSDGKTYLN |
| 9 | 5G6 CDRL2 | LASKLDS |
| 10 | 5G6 CDRL3 | WQATHFPWT |
| 11 | 5G6—Light | GATGTTGTGATGACCCAGACTCCACTCACTTTGTCGGTTACCATTGG |
| | chain variable | ACAACCAGCCTCCATCTCTTGCAAGTCAAGTCAGAGCCTCTTAGATA |
| | region cDNA | GTGATGGAAAGACATATTTGAATTGGTTGTTACAGAGGCCTGGCCA |
| | | GTCTCCAAAGCGCCTAATCTATCTGGCGTCTAAACTGGACTCTGGAG |
| | | TCCCTGACAGGTTCACTGGCAGTGGATCAGGGACAGATTTCACACT |
| | | GAAAATCAGCAGAGTGGAGGCTGAGGATTTGGGAGTTTATTATTGC |
| | | TGGCAAGCTACACATTTTCCGTGGACGTTCGGTGGAGGCACCAAGC |
| | | TGGAAATCAAA |
| 12 | 5G6 heavy chain | EVQLQQSGAELARPGASVKMSCKASGYTFTSYWMHWVKQRPGQGLE |
| | variable region | WIGAIYPGNSDISYNQKFKGKAKLTAVTSATTAYMELSSLTNEDSAVY |
| | | YCTLYDYDPDYWGQGTTLTVSS |
| 13 | 5G6 CDRH1 | SYWMH |
| 14 | 5G6 CDRH2 | AIYPGNSDISYNQKFKG |
| 15 | 5G6 CDRH3 | YDYDPDY |
| 16 | 5G6—Heavy | GAGGTCCAACTGCAGCAGTCTGGGGCTGAGCTGGCAAGACCTGGGG |
| | chain variable | CTTCAGTGAAGATGTCCTGCAAGGCTTCTGGCTACACCTTTACCAGC |
| | region cDNA | TACTGGATGCACTGGGTAAAACAGAGGCCTGGACAGGGTCTGGAAT |
| | | GGATTGGCGCTATTTATCCTGGAAATAGTGATATTAGCTACAATCAG |
| | | AAGTTCAAGGGCAAGGCCAAACTGACTGCAGTCACATCCGCCACCA |
| | | CTGCCTACATGGAGCTCAGCAGCCTAACAAATGAGGACTCTGCGGT |
| | | CTATTACTGTACCCTCTATGATTACGACCCTGACTACTGGGGCCAAG |
| | | GCACCACTCTCACAGTCTCCTCA |
| 17 | 1A8 light chain | DIVMTQSPSSLAMSVGQKVTMNCKSSQSLLNSSNQKNYLAWFQQKPG |
| | variable region | QSPKLLVYFASTRESGVPDRFIGSGSGTDFTLTISSVQAEDLADYFCQQ |
| | | HYSTPLTFGAGTKLELK |
| 18 | 1A8 CDRL 1 | KSSQSLLNSSNQKNYLA |
| 19 | 1A8 CDRL2 | FASTRES |
| 20 | 1A8 CDRL3 | QQHYSTPLT |
| 21 | 1A8 light chain | GACATTGTGATGACACAGTCTCCATCCTCCCTGGCTATGTCAGTAGG |
| | variable region | ACAGAAGGTCACTATGAACTGCAAGTCCAGTCAGAGCCTTTTAAAT |
| | cDNA | AGTAGCAATCAAAAGAACTATTTGGCCTGGTTCCAGCAGAAACCAG |
| | | GACAGTCTCCTAAACTTCTGGTATACTTTGCTTCCACTAGGGAATCT |
| | | GGGGTCCCTGATCGCTTCATAGGCAGTGGATCTGGGACAGATTTCAC |
| | | TCTTACCATCAGCAGTGTGCAGGCTGAAGACCTGGCAGATTACTTCT |
| | | GTCAGCAACATTATAGCACTCCTCTCACGTTCGGTGCTGGGACCAAG |
| | | CTGGAGCTGAAA |
| 22 | 1A8 heavy chain | EVQLQQSGAELVRPGALVKLSCKASGFNIKDYYMHWVKQRPEQGLE |
| | variable region | WIGWIDPENGNTIYDPKFQGKATITADTSSNTAYLQLSSLASEDTAVY |
| | | YCARGWLLLWGQGTTLTVSS |
| 23 | 1A8 CDRH1 | DYYMH |
| 24 | 1A8 CDRH2 | WIDPENGNTIYDPKFQG |
| 25 | 1A8 CDRH3 | GWLLL |
| 26 | 1A8 heavy chain | GAGGTTCAGCTGCAGCAGTCTGGGGCTGAGCTTGTGAGGCCAGGGG |
| | variable region | CCTTAGTCAAGTTGTCCTGCAAAGCTTCTGGCTTCAACATTAAAGAC |
| | cDNA | TACTATATGCACTGGGTGAAGCAGAGGCCTGAACAGGGCCTGGAGT |
| | | GGATTGGATGGATTGATCCTGAGAATGGTAATACTATATATGACCC |
| | | GAAGTTCCAGGGCAAGGCCACTATAACAGCAGACACATCCTCCAAC |
| | | ACAGCCTACCTGCAGCTCAGCAGCCTGGCATCTGAGGACACTGCCG |
| | | TCTATTACTGTGCTAGAGGATGGTTACTACTTTGGGGCCAAGGCACC |
| | | ACTCTCACAGTCTCCTCA |
| 27 | 4B3 light chain | EIVLTQSPALMAASPGEKVTITCSVSSSISSSNLHWYQQKSETSPKPWIY |
| | variable region | GTSNLASGVPVRFSGSGSGTSYSLTISSMEAEDAATYYCQQWSSYPLT |
| | | FGAGTKLELE |
| 28 | 4B3 CDRL1 | SVSSSISSSNLH |
| 29 | 4B3 CDRL2 | GTSNLAS |
| 30 | 4B3 CDRL3 | QQWSSYPLT |
| 31 | 4B3 light chain | GAAATTGTGCTCACCCAGTCTCCAGCACTCATGGCTGCATCTCCAGG |
| | variable region | GGAGAAGGTCACCATCACCTGCAGTGTCAGCTCAAGTATAAGTTCC |
| | cDNA | AGCAACTTGCACTGGTACCAGCAGAAGTCAGAAACCTCCCCCAAAC |
| | | CCTGGATTTATGGCACATCCAACCTGGCTTCTGGAGTCCCTGTTCGC |
| | | TTCAGTGGCAGTGGATCTGGGACCTCTTATTCTCTCACAATCAGCAG |
| | | CATGGAGGCTGAAGATGCTGCCACTTATTACTGTCAACAGTGGAGT |
| | | AGTTACCCACTCACGTTCGGTGCTGGGACCAAGCTGGAACTGGAA |
| 32 | 4B3 heavy chain | EVQLQQSGPELVKPGSSVKISCKASGYTFTDYNMDWVKQSHGKSLEWI |
| | variable region | GTINPNNGGTSYNQKFKGKATLTVDKSSNTAYMELRSLTSEDSAVYY |
| | | CARGYDYDLWFAYWGQGTLVTVSA |
| 33 | 4B3 CDRH1 | DYNMD |
| 34 | 4B3 CDRH2 | TINPNNGGTSYNQKFKG |
| 35 | 4B3 CDRH3 | GYDYDLWFAY |
| 36 | 4B3 heavy chain | GAGGTCCAGCTGCAACAGTCTGGACCTGAGCTGGTGAAGCCTGGGT |
| | variable region | CTTCAGTGAAGATATCCTGCAAAGCTTCTGGATACACATTCACTGAC |
| | cDNA | TACAACATGGACTGGGTGAAGCAGAGCCATGGAAAGAGCCTTGAGT |
| | | GGATTGGTACTATTAATCCTAACAATGGTGGTACTAGCTACAACCAG |
| | | AAGTTCAAGGGCAAGGCCACATTGACTGTAGACAAGTCCTCCAACA |
| | | CAGCCTACATGGAGCTCCGCAGCCTGACATCTGAGGACTCTGCAGT |
| | | CTATTACTGTGCAAGAGGCTATGATTACGACTTGTGGTTTGCTTACT |
| | | GGGGCCAAGGGACTCTGGTCACTGTCTCTGCA |
| 37 | 4E11 light chain | DILMTQSPSSMSVSLGDTVSITCHASQGINSNIGWLLQKPGKSFKGLIY |
| | variable region | HGTNLEDGVPSRFSGSGSGTDYSLTISSLESEDFADYYCVQYAQFPYTF |
| | | GGGTKLEIK |
| 38 | 4E11 CDRL1 | HASQGINSNIG |
| 39 | 4E11 CDRL2 | HGTNLED |
| 40 | 4E11 CDRL3 | VQYAQFPYT |
| 41 | 4E11 light chain | GACATCCTGATGACCCAATCTCCATCCTCCATGTCTGTATCTCTGGG |
| | variable region | AGACACAGTCAGCATCACTTGCCATGCAAGTCAGGGCATTAACAGT |
| | cDNA | AATATAGGGTGGTTGCTGCAGAAACCAGGGAAATCATTTAAGGGCC |
| | | TGATCTATCATGGAACCAACTTGGAAGATGGAGTTCCATCAAGGTTC |
| | | AGTGGCAGTGGATCTGGAACAGATTATTCTCTCACCATCAGCAGCCT |
| | | GGAATCTGAGGATTTTGCTGACTATTACTGTGTACAGTATGCTCAGT |
| | | TTCCGTACACGTTCGGAGGGGGGACCAAACTGGAAATAAAA |
| 42 | 4E11 heavy | DVQLQESGPGLVKPSQSLSLTCTVTGYSITSDYAWNWIRQFPGNKLEW |
| | chain variable | MGYIGYNGRTSYNPSLKSRISITRDTSKNQFFLQLNYVTTEDTATFYCA |
| | region | RLGRGFAYWGQGTLVTVSA |
| 43 | 4E11 CDRH1 | SDYAWN |
| 44 | 4E11 CDRH2 | YIGYNGRTSYNPSLKS |
| 45 | 4E11 CDRH3 | LGRGFAY |
| 46 | 4E11 heavy | GATGTGCAGCTTCAGGAGTCGGGACCTGGCCTGGTGAAACCTTCTC |
| | chain variable | AGTCTCTGTCCCTCACCTGCACTGTCACTGGCTACTCAATCACCAGT |
| | region cDNA | GATTATGCCTGGAACTGGATCCGGCAGTTTCCAGGAAACAAACTGG |
| | | AGTGGATGGGCTACATAGGCTACAATGGTAGAACTAGTTACAACCC |
| | | ATCTCTCAAAAGTCGAATCTCTATCACTCGAGACACATCCAAGAACC |
| | | AGTTCTTCCTGCAGTTGAATTATGTGACTACTGAGGACACAGCCACA |
| | | TTTTACTGTGCAAGACTGGGCCGAGGGTTTGCTTACTGGGGCCAAGG |
| | | GACTCTGGTCACTGTCTCTGCA |
| 47 | 5D8 light chain | EIVLTQSPVFMAASPGEKVTITCSVSSSISSSNLHWYQQKSETSPKPWIY |
| | variable region | GTSNLASGVPVRFSGSGSGTSYSLTISSMEAEDAATYYCQQWSSYPLT |
| | | FGAGTKLELK |
| 48 | 5D8 CDRL 1 | SVSSSISSSNLH |
| 49 | 5D8 CDRL2 | GTSNLAS |
| 50 | 5D8 CDRL3 | QQWSSYPLT |
| 51 | 5D8 light chain | GAAATTGTGCTCACCCAGTCTCCAGTATTCATGGCTGCATCTCCAGG |
| | variable region | GGAGAAGGTCACCATCACCTGCAGTGTCAGCTCAAGTATAAGTTCC |
| | cDNA | AGCAACTTGCACTGGTACCAGCAGAAGTCAGAAACCTCCCCCAAAC |
| | | CCTGGATTTATGGCACATCCAACCTGGCTTCTGGAGTCCCTGTTCGC |
| | | TTCAGTGGCAGTGGATCTGGGACCTCTTATTCTCTCACAATCAGCAG |
| | | CATGGAGGCTGAAGATGCTGCCACTTATTACTGTCAACAGTGGAGT |
| | | AGTTACCCACTCACGTTCGGTGCTGGGACCAAGCTGGAGCTGAAA |
| 52 | 5D8 heavy chain | EVQLQQSGPDLVKPGSSVKISCKASGYTFTDYNIDWVKQSHGKSLEWI |
| | variable region | GTINPNYGGTSYNQKFKGKATLTVDKSSSTAYMELRSLTSEDSAVYY |
| | | CARGYDYDLWFAYWGQGTLVTVSA |
| 53 | 5D8 CDRH1 | DYNID |
| 54 | 5D8 CDRH2 | TINPNYGGTSYNQKFKG |
| 55 | 5D8 CDRH3 | GYDYDLWFAY |
| 56 | 5D8 heavy chain | GAGGTCCAGCTGCAACAGTCTGGACCTGACCTGGTGAAGCCTGGGT |
| | variable region | CTTCAGTGAAGATTTCCTGCAAAGCTTCTGGATACACATTCACTGAC |
| | cDNA | TACAACATTGACTGGGTGAAGCAGAGCCATGGAAAGAGCCTTGAGT |
| | | GGATTGGAACTATTAATCCTAACTATGGTGGTACTTCCTACAACCAG |
| | | AAGTTCAAGGGCAAGGCCACATTGACTGTAGACAAGTCCTCCAGCA |
| | | CAGCCTACATGGAGCTCCGCAGCCTGACATCTGAGGACTCTGCAGT |
| | | CTATTACTGTGCAAGAGGCTATGATTACGACTTGTGGTTTGCTTACT |
| | | GGGGCCAAGGGACTCTGGTCACTGTCTCTGCA |
| 57 | 9C9 light chain | EIVLTQSPTLMAASPGEKVTITCSVSSSISSSNLHWYQQKSETSPKPWIY |
| | variable region | DTSNLASGVPIRFSGSGSGTSYSLTISSVEAEDAATYYCQQWSSYPLTF |
| | | GSGTKLEIK |
| 58 | 9C9 CDRL 1 | SVSSSISSSNLH |
| 59 | 9C9 CDRL2 | DTSNLAS |
| 60 | 9C9 CDRL3 | QQWSSYPLT |
| 61 | 9C9 light chain | GAAATTGTGCTCACCCAGTCTCCAACACTCATGGCTGCATCTCCAGG |
| | variable region | GGAGAAGGTCACCATCACCTGCAGTGTCAGCTCAAGTATAAGTTCC |
| | cDNA | AGCAACTTGCACTGGTACCAGCAGAAGTCAGAAACCTCCCCCAAAC |
| | | CCTGGATTTATGACACATCCAACCTGGCTTCTGGAGTCCCTATTCGC |
| | | TTCAGTGGCAGTGGATCTGGGACCTCTTATTCTCTCACAATCAGCAG |
| | | CGTGGAGGCTGAAGATGCTGCCACTTATTACTGTCAACAGTGGAGT |
| | | AGTTACCCACTCACGTTCGGCTCGGGGACAAAGTTGGAAATAAAA |
| 62 | 9C9 heavy chain | QVQLQQPGAELVKPGASVKLSCKASGYTFTSYWMHWVKQRPGQDLE |
| | variable region | WIGEIDPSDSYTNYNQKFKGKATLTVDKSSSTAYIQLSSLTSEDSALYY |
| | | CARFDFAYWGQGTLVTVSA |
| 63 | 9C9 CDRH1 | SYWMH |
| 64 | 9C9 CDRH2 | EIDPSDSYTNYNQKFKG |
| 65 | 9C9 CDRH3 | FDFAY |
| 66 | 9C9 heavy chain | CAGGTCCAACTGCAGCAGCCTGGGGCTGAGCTTGTGAAGCCTGGGG |
| | variable region | CTTCAGTGAAGCTGTCCTGCAAGGCTTCTGGCTACACCTTCACCAGC |
| | cDNA | TACTGGATGCACTGGGTGAAACAGAGGCCTGGACAAGACCTTGAGT |
| | | GGATCGGAGAGATTGATCCTTCTGATAGTTATACTAACTACAATCAA |
| | | AAGTTCAAGGGCAAGGCCACATTGACTGTAGACAAATCCTCCAGCA |
| | | CAGCCTACATTCAGCTCAGCAGCCTGACATCTGAGGACTCTGCGCTC |
| | | TATTACTGTGCAAGATTCGATTTTGCTTACTGGGGCCAAGGGACTCT |
| | | GGTCACTGTCTCTGCA |
| 67 | 11B1 light chain | DVVMTQTPLSLPVSLGDQASISCRSSQSLVYSNGNTYLHWYLQKPGQS |
| | 1 (L1) variable | PKLLIYKVSNRFSGVPDRFSGSGSGTDFTLKISRVEAEDLGVYFCSQST |
| | region | HVPFTFGSGTKLEIK |
| | (dominant) | |
| 68 | 11B1 L1 CDRL1 | RSSQSLVYSNGNTYLH |
| 69 | 11B1 L1 CDRL2 | KVSNRFS |
| 70 | 11B1 L1 CDRL3 | SQSTHVPFT |
| 71 | 11B1 light chain | GATGTTGTGATGACCCAAACTCCACTCTCCCTGCCTGTCAGTCTTGG |
| | 1 (L1) variable | AGATCAAGCCTCCATCTCTTGCAGATCTAGTCAGAGCCTTGTATATA |
| | region cDNA | GTAATGGAAACACCTATTTACATTGGTACCTGCAGAAGCCAGGCCA |
| | (dominant) | GTCTCCAAAGCTCCTGATCTACAAAGTTTCCAACCGATTTTCTGGGG |
| | | TCCCAGACAGGTTCAGTGGCAGTGGATCAGGGACAGATTTCACACT |
| | | CAAGATCAGCAGAGTGGAGGCTGAGGATCTGGGAGTTTATTTCTGC |
| | | TCTCAAAGTACACATGTTCCATTCACGTTCGGCTCGGGGACAAAGTT |
| | | GGAAATAAAA |
| 72 | 11B1 light chain | ENVLTQSPAIMSASLGEKVTMSCRASSSVNYMYWCQQKSDASPKLWI |
| | 2 (L2) variable | YYTSNLAPGVPARFSGSGSGNSYSLTISSMEGEDVATYYCQQFTSSPS |
| | region | MHTFGGGTKLEIK |
| 73 | 11B1 L2 CDRL 1 | RASSSVNYMY |
| 74 | 11B1 L2 CDRL2 | YTSNLAP |
| 75 | 11B1 L2 CDRL3 | QQFTSSPSMHT |
| 76 | 11B1 light chain | GAAAATGTGCTCACCCAGTCTCCAGCAATCATGTCTGCATCTCTAGG |
| | 2 (L2) variable | GGAGAAGGTCACCATGAGCTGCAGGGCCAGCTCAAGTGTAAATTAC |
| | region cDNA | ATGTACTGGTGCCAGCAGAAGTCAGATGCCTCCCCCAAACTATGGA |
| | | TTTATTACACATCCAACCTGGCTCCTGGAGTCCCAGCTCGCTTCAGT |
| | | GGCAGTGGGTCTGGGAACTCTTATTCTCTCACAATCAGCAGCATGGA |
| | | GGGTGAAGATGTTGCCACTTATTACTGCCAGCAGTTTACTAGTTCCC |
| | | CATCCATGCACACGTTCGGAGGGGGGACCAAGCTGGAAATAAAA |
| 77 | 11B1 heavy | QIQLVQSGPELKKPGETVKISCKASGYTFTTAGMQWVKKMPGKGFKW |
| | chain variable | IGWINTHSGDPKYAEDFKGRFAFSLETYASTAYLQISNLKNEDTASYF |
| | region | CARTHIYDGYNYAMDYWGQGTSVTVSS |
| 78 | 11B1 CDRH1 | TAGMQ |
| 79 | 11B1 CDRH2 | WINTHSGDPKYAEDFKG |
| 80 | 11B1 CDRH3 | THIYDGYNYAMDY |
| 81 | 11B1 heavy | CAGATCCAGTTGGTGCAGTCTGGACCTGAGCTGAAGAAGCCTGGAG |
| | chain variable | AGACAGTCAAGATCTCCTGCAAGGCTTCTGGGTATACCTTCACAACT |
| | region cDNA | GCTGGAATGCAGTGGGTAAAAAAGATGCCAGGAAAGGGTTTTAAGT |
| | | GGATTGGCTGGATAAACACCCACTCTGGAGATCCAAAATATGCAGA |
| | | AGACTTCAAGGGACGGTTTGCCTTCTCTTTGGAAACCTACGCCAGTA |
| | | CTGCATATTTGCAGATAAGCAACCTCAAAAACGAGGACACTGCTTC |
| | | GTATTTCTGTGCGAGGACCCACATCTATGATGGTTATAACTATGCTA |
| | | TGGACTACTGGGGTCAAGGGACCTCAGTCACCGTCTCCTCA |
| 82 | 11C8 light chain | DVVMTQTPLSLPVSLGDQASISCRSSQSLVYSNGNTYLHWYLQKPGQS |
| | 1 (L1) variable | PKLLIYKVSNRFSGVPDRFSGSGSGTDFTLKISRVEAEDLGVYFCSQST |
| | region | HVPFTFGSGTKLEIK |
| | (dominant) | |
| 83 | 11C8 L1 CDRL1 | RSSQSLVYSNGNTYLH |
| 84 | 11C8 L1 CDRL2 | KVSNRFS |
| 85 | 11C8 L1 CDRL3 | SQSTHVPFT |
| 86 | 11C8 light chain | GATGTTGTGATGACCCAAACTCCACTCTCCCTGCCTGTCAGTCTTGG |
| | 1 (L1) variable | AGATCAAGCCTCCATCTCTTGCAGATCTAGTCAGAGCCTTGTATATA |
| | region cDNA | GTAATGGAAACACCTATTTACATTGGTACCTGCAGAAGCCAGGCCA |
| | (dominant) | GTCTCCAAAGCTCCTGATCTACAAAGTTTCCAACCGATTTTCTGGGG |
| | | TCCCAGACAGGTTCAGTGGCAGTGGATCAGGGACAGATTTCACACT |
| | | CAAGATCAGCAGAGTGGAGGCTGAGGATCTGGGAGTTTATTTCTGC |
| | | TCTCAAAGTACACATGTTCCATTCACGTTCGGCTCGGGGACAAAGTT |
| | | GGAAATAAAA |
| 87 | 11C8 light chain | ENVLTQSPAIMSASLGEKVTMSCRASSSVNYMYWCQQKSDASPKLWI |
| | 2 (L2) variable | YYTSNLAPGVPARFSGSGSGNSYSLTISSMEGEDVATYYCQQFTSSPS |
| | region | MHTFGGGTKLEIK |
| 88 | 11C8 L2 CDRL1 | RASSSVNYMY |
| 89 | 11C8 L2 CDRL2 | YTSNLAP |
| 90 | 11C8 L2 CDRL3 | QQFTSSPSMHT |
| 91 | 11C8 light chain | GAAAATGTGCTCACCCAGTCTCCAGCAATCATGTCTGCATCTCTAGG |
| | 2 (L2) variable | GGAGAAGGTCACCATGAGCTGCAGGGCCAGCTCAAGTGTAAATTAC |
| | region cDNA | ATGTACTGGTGCCAGCAGAAGTCAGATGCCTCCCCCAAACTATGGA |
| | | TTTATTACACATCCAACCTGGCTCCTGGAGTCCCAGCTCGCTTCAGT |
| | | GGCAGTGGGTCTGGGAACTCTTATTCTCTCACAATCAGCAGCATGGA |
| | | GGGTGAAGATGTTGCCACTTATTACTGCCAGCAGTTTACTAGTTCCC |
| | | CATCCATGCACACGTTCGGAGGGGGGACCAAGCTGGAAATAAAA |
| 92 | 11C8 heavy | QIQLVQSGPELKKPGETVKISCKASGYTFTTAGMQWVQKMPGKGFKW |
| | chain variable | IGWINTHSGDPKYAEDFKGRFAFSLETYASTAYLQISNLKNEDTASYF |
| | region | CARTHIYDGYNYAMDYWGQGTSVTVSS |
| 93 | 11C8 CDRH1 | TAGMQ |
| 94 | 11C8 CDRH2 | WINTHSGDPKYAEDFKG |
| 95 | 11C8 CDRH3 | THIYDGYNYAMDY |
| 96 | 11C8 heavy | CAGATCCAGTTGGTGCAGTCTGGACCTGAGCTGAAGAAGCCTGGAG |
| | chain variable | AGACAGTCAAGATCTCCTGCAAGGCTTCTGGGTATACCTTCACAACT |
| | region cDNA | GCTGGAATGCAGTGGGTACAAAAGATGCCAGGAAAGGGTTTTAAGT |
| | | GGATTGGCTGGATAAACACCCACTCTGGAGATCCAAAATATGCAGA |
| | | AGACTTCAAGGGACGGTTTGCCTTCTCTTTGGAAACCTACGCCAGTA |
| | | CTGCATATTTGCAGATAAGCAACCTCAAAAACGAGGACACTGCTTC |
| | | GTATTTCTGTGCGAGGACCCACATCTATGATGGTTACAACTATGCTA |
| | | TGGACTACTGGGGTCAAGGGACCTCAGTCACCGTCTCCTCA |
| 97 | 11H3 light chain | DVVMTQTPLSLPVSLGDQASISCRSSQSLVYSNGNTYLHWYLQKPGQS |
| | variable region | PKLLIYKVSNRFSGVPDRFSGSGSGTDFTLKISRVEAEDLGVYFCSQST |
| | | HVPFTFGSGTKLEIK |
| 98 | 11H3 CDRL1 | RSSQSLVYSNGNTYLH |
| 99 | 11H3 CDRL2 | KVSNRFS |
| 100 | 11H3 CDRL3 | SQSTHVPFT |
| 101 | 11H3 light chain | GATGTTGTGATGACCCAAACTCCACTCTCCCTGCCTGTCAGTCTTGG |
| | variable region | AGATCAAGCCTCCATCTCTTGCAGATCTAGTCAGAGCCTTGTATATA |
| | cDNA | GTAATGGAAACACCTATTTACATTGGTACCTGCAGAAGCCAGGCCA |
| | | GTCTCCAAAGCTCCTGATCTACAAAGTTTCCAACCGATTTTCTGGGG |
| | | TCCCAGACAGGTTCAGTGGCAGTGGATCAGGGACAGATTTCACACT |
| | | CAAGATCAGCAGAGTGGAGGCTGAGGATCTGGGAGTTTATTTCTGC |
| | | TCTCAAAGTACACATGTTCCATTCACGTTCGGCTCGGGGACAAAGTT |
| | | GGAAATAAAA |
| 102 | 11H3 heavy | QIQLVQSGPELKKPGETVKISCKASGYTFTTAGMQWVQKMPGKGFKW |
| | chain variable | IGWINTHSGDPKYAEDFKGRFAFSLETYASTAYLQISNLKNEDTATYF |
| | region | CARTHIYDGYNYAMDYWGQGTSVTVSS |
| 103 | 11H3 CDRH1 | TAGMQ |
| 104 | 11H3 CDRH2 | WINTHSGDPKYAEDFKG |
| 105 | 11H3 CDRH3 | THIYDGYNYAMDY |
| 106 | 11H3 heavy | CAGATCCAGTTGGTGCAGTCTGGACCTGAGCTGAAGAAGCCTGGAG |
| | chain variable | AGACAGTCAAGATCTCCTGCAAGGCTTCTGGGTATACCTTCACAACT |
| | region cDNA | GCTGGAATGCAGTGGGTACAAAAGATGCCAGGAAAGGGTTTTAAGT |
| | | GGATTGGCTGGATAAACACCCACTCTGGAGATCCAAAATATGCAGA |
| | | AGACTTCAAGGGACGGTTTGCCTTCTCTTTGGAAACCTACGCCAGCA |
| | | CTGCATATTTGCAGATAAGCAACCTCAAAAACGAGGACACTGCTAC |
| | | GTATTTCTGTGCGAGGACCCATATCTATGATGGTTATAATTATGCTA |
| | | TGGACTACTGGGGTCAAGGAACCTCAGTCACCGTCTCCTCA |
| 107 | 4E11 Heavy | DVQLQESGPGLVKPSQSLSLTCTVTGYSITSDYAWNWIRQFPGNKLEW |
| | chain human | MGYIGYNGRTSYNPSLKSRISITRDTSKNQFFLQLNYVTTEDTATFYCA |
| | IgG1 | RLGRGFAYWGQGTLVTVSAASTKGPSVFPLAPSSKSTSGGTAALGCLV |
| | (variable region | KDYFPEPVTVSWNSGALTSGVHTFPAVLQSSGLYSLSSVVTVPSSSLGT |
| | underlined) | QTYICNVNHKPSNTKVDKKVEPKSCDKTHTCPPCPAPELLGGPSVFLFP |
| | | PKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKA |
| | | KGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNH |
| | | YTQKSLSLSPG |
| 108 | 4E11 Light chain | DILMTQSPSSMSVSLGDTVSITCHASQGINSNIGWLLQKPGKSFKGLIYH |
| | human Kappa | GTNLEDGVPSRFSGSGSGTDYSLTISSLESEDFADYYCVQYAQFPYTFG |
| | (variable region | GGTKLEIKRTVAAPSVFIFPPSDEQLKSGTASVVCLLNNFYPREAKVQW |
| | underlined) | KVDNALQSGNSQESVTEQDSKDSTYSLSSTLTLSKADYEKHKVYACEV |
| | | THQGLSSPVTKSFNRGEC |
| 109 | 5G6 Heavy chain | EVQLQQSGAELARPGASVKMSCKASGYTFTSYWMHWVKQRPGQGLE |
| | human IgG1 | WIGAIYPGNSDISYNQKFKGKAKLTAVTSATTAYMELSSLTNEDSAVY |
| | (variable region | YCTLYDYDPDYWGQGTTLTVSSASTKGPSVFPLAPSSKSTSGGTAALG |
| | underlined) | CLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSVF |
| | | LFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTIS |
| | | KAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPG |
| 110 | 5G6 Light chain | DVVMTQTPLTLSVTIGQPASISCKSSQSLLDSDGKTYLNWLLQRPGQSP |
| | human Kappa | KRLIYLASKLDSGVPDRFTGSGSGTDFTLKISRVEAEDLGVYYCWQAT |
| | (variable region | HFPWTFGGGTKLEIKRTVAAPSVFIFPPSDEQLKSGTASVVCLLNNFYPR |
| | underlined) | EAKVQWKVDNALQSGNSQESVTEQDSKDSTYSLSSTLTLSKADYEKH |
| | | KVYACEVTHQGLSSPVTKSFNRGEC |
| 111 | 13.1.2 Heavy | QVOLVESGGGVVQPGRSLRLSCAASGFTFSSYGMHWVRQAPGKGLEW |
| | chain human | VAVIWYDGSNKYYVDSVKGRFTISRDNSKNTLYLQMNSLRAEDTAVY |
| | IgG1 | YCARDGWQQLAPFDYWGQGTLVTVSSASTKGPSVFPLAPSSKSTSGGT |
| | (variable region | AALGCLVKDYFPEPVTVSWNSGALTSGVHTFPAVLQSSGLYSLSSVVT |
| | underlined) | VPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPG |
| 112 | 13.1.2 Light | DIVMTQTPLSSPVTLGQPASISCRSSQSLVHSDGNTYLSWLHQRPGQPPR |
| | chain human | LLIYKISNRFSGVPDRFSGSGAGTAFTLKISRVEAEDVGVYYCMQATQL |
| | Kappa | PRTFGQGTKVEIKRTVAAPSVFIFPPSDEQLKSGTASVVCLLNNFYPREA |
| | (variable region | KVQWKVDNALQSGNSQESVTEQDSKDSTYSLSSTLTLSKADYEKHKV |
| | underlined) | YACEVTHQGLSSPVTKSFNRGEC |
| 113 | N-terminal light | MVLQTQVFISLLLWISGAYG |
| | chain signal | |
| | sequence | |
| 114 | N-terminal heavy | MDWTWRILFLVAAATGTHA |
| | chain signal | |
| | sequence | |
| 115 | consensus VLs | EIVLTQSPX₁ₐX₂ₐMAASPGEKVTITCSVSSSISSSNLHWYQQKSETSPKPW |
| | of 4B3 and 5D8 | IYGTSNLASGVPVRFSGSGSGTSYSLTISSMEAEDAATYYCQQWSSYPL |
| | | TFGAGTKLELX₃ₐ |
| | | Where X₁ₐ may be a conservative amino acid substitution |
| | | to alanine (A) or valine (V) or is alanine or valine |
| | | Where X₂ₐ may be a conservative amino acid substitution |
| | | to leucine (L) or phenylalanine (F) or is leucine |
| | | or phenylalanine |
| | | Where X₃ₐ may be a conservative amino acid substitution |
| | | to lysine (K) or glutamic acid (E) or is lysine or |
| | | glutamic acid |
| 116 | consensus VHs | EVQLQQSGPX_{1b}LVKPGSSVKISCKASGYTFTDYNX_{2b}DWVKQSHGKSLE |
| | of 4B3 and 5D8 | WIGTINPNX_{3b}GGTSYNQKFKGKATLTVDKSSX_{4b}TAYMELRSLTSEDS |
| | | AVYYCARGYDYDLWFAYWGQGTLVTVSA |
| | | Where X_{1b} may be a conservative amino acid substitution |
| | | to glutamic acid (E) or aspartic acid (D) or is glutamic |
| | | acid or aspartic acid |
| | | Where X_{2b} may be a conservative amino acid substitution to |
| | | methionine (M) or isoleucine (I) or is methionine or |
| | | isoleucine |
| | | Where X_{3b} may be a conservative amino acid substitution to |
| | | asparagine (N) or tyrosine (Y) or is asparagine or tyro- |
| | | sine |
| | | Where X_{4b} may be a conservative amino acid substitution to |
| | | asparagine (N) or serine (S) or is asparagine or serine |
| 117 | Consensus of | QIQLVQSGPELKKPGETVKISCKASGYTFTTAGMQWVX_{1c}KMPGKGFK |
| | VHs of 11B1, | WIGWINTHSGDPKYAEDFKGRFAFSLETYASTAYLQISNLKNEDTAX_{2c} |
| | 11C8 and 11H3 | YFCARTHIYDGYNYAMDYWGQGTSVTV |
| | | Where X_{1c} may be a conservative amino acid substitution to |
| | | lysine (K) or glutamine (Q) or is lysine or glutamine |
| | | Where X_{2c} may be conservative amino acid substitution to |
| | | serine (S) or threonine (T) or is serine or threonine |
| 118 | consensus VLs | EIVLTQSPX_{1d}X_{2d}MAASPGEKVTITCSVSSSISSSNLHWYQQKSETSPKP |
| | of 4B3, 5D8 and | WIYX_{3d}TSNLASGVPX_{4d}RFSGSGSGTSYSLTISSXEAEDAATYYCQQW |
| | 9C9 | SSYPLTFGX_{6d}GTKLEX_{7d}X_{8d} |
| | | Where X_{1d} may be a conservative amino acid substitution to |
| | | alanine (A), threonine (T) or valine (V) or is alanine, |
| | | threonine or valine |
| | | Where X_{2d} may be a conservative amino acid substitution to |
| | | leucine (L) or phenylalanine (F) or is leucine or phenyl- |
| | | alanine |
| | | Where X_{3d} may be a conservative amino acid substitution to |
| | | glycine (G) or aspartic acid (D) or is glycine or |
| | | glutamic acid |
| | | Where X_{4d} may be a conservative amino acid substitution to |
| | | isoleucine (I) or valine (V) or is isoleucine or valine |
| | | Where X_{5d} may be a conservative amino acid substitution to |
| | | methionine (M) or valine (V) or is methionine or valine |
| | | Where X_{6d} may be a conservative amino acid substitution to |
| | | alanine (A) or serine (S) or is alanine or serine |
| | | Where X_{7d} may be a conservative amino acid substitution to |
| | | isoleucine (I) or leucine (L) or is isoleucine or leucine |
| | | Where X_{8d} may be a conservative amino acid substitution to |
| | | lysine (K) or glutamic acid (E) or is lysine or glutamic |
| | | acid |
| 119 | Amino acid residues 1 to 76 of | LEEKKGNYVVTDHGSCVRACGADSYEMEEDGVRKC |
| | EGFRvIII | KKCEGPCRKVCNGIGIGEFKDSLSINATNIKHFKNCTSI |
| | | SG |
| 120 | Amino acid residues 1 to 62 of | LEEKKGNYVVTDHGSCVRACGADSYEMEEDGVRKC |
| | EGFRvIII | KKCEGPCRKVCNGIGIGEFKDSLSINA |
| 121 | Amino acid residues 1 to 49 of | LEEKKGNYVVTDHGSCVRACGADSYEMEEDGVRKC |
| | EGFRvIII | KKCEGPCRKVCNGI |
| 122 | Amino acid residues 1 to 45 of | LEEKKGNYVVTDHGSCVRACGADSYEMEEDGVRKC |
| | EGFRvIII | KKCEGPCRKV |
| 123 | Amino acid residues 1 to 37 of | LEEKKGNYVVTDHGSCVRACGADSYEMEEDGVRKC |
| | EGFRvIII | KK |
| 124 | Amino acid residues 1 to 33 of | LEEKKGNYVVTDHGSCVRACGADSYEMEEDGVR |
| | EGFRvIII | |
| 125 | Amino acid residues 1 to 18 of | LEEKKGNYVVTDHGSCVR |
| | EGFRvIII | |
| 126 | Amino acid residues 3 to 49 of | EKKGNYVVTDHGSCVRACGADSYEMEEDGVRKCKK |
| | EGFRvIII | CEGPCRKVCNGI |
| 127 | Amino acid residues 3 to 45 of | EKKGNYVVTDHGSCVRACGADSYEMEEDGVRKCKK |
| | EGFRvIII | CEGPCRKV |
| 128 | Amino acid residues 3 to 37 of | EKKGNYVVTDHGSCVRACGADSYEMEEDGVRKCKK |
| | EGFRvIII | |
| 129 | Amino acid residues 3 to 18 of | EKKGNYVVTDHGSCVR |
| | EGFRvIII | |
| 130 | Amino acid residues 6 to 49 of | GNYVVTDHGSCVRACGADSYEMEEDGVRKCKKCEG |
| | EGFRvIII | PCRKVCNGI |
| 131 | Amino acid residues 6 to 45 of | GNYVVTDHGSCVRACGADSYEMEEDGVRKCKKCEG |
| | EGFRvIII | PCRKV |
| 132 | Amino acid residues 6 to 37 of | GNYVVTDHGSCVRACGADSYEMEEDGVRKCKK |
| | EGFRvIII | |
| 133 | Amino acid residues 10 to 49 of | VTDHGSCVRACGADSYEMEEDGVRKCKKCEGPCRK |
| | EGFRvIII | VCNGI |
| 134 | Amino acid residues 10 to 45 of | VTDHGSCVRACGADSYEMEEDGVRKCKKCEGPCRK |
| | EGFRvIII | V |
| 135 | Amino acid residues 10 to 37 of | VTDHGSCVRACGADSYEMEEDGVRKCKK |
| | EGFRvIII | |
| 136 | Amino acid residues 15 to 49 of | SCVRACGADSYEMEEDGVRKCKKCEGPCRKVCNGI |
| | EGFRvIII | |
| 137 | Amino acid residues 15 to 45 of | SCVRACGADSYEMEEDGVRKCKKCEGPCRKV |
| | EGFRvIII | |
| 138 | Amino acid residues 19 to 76 of | ACGADSYEMEEDGVRKCKKCEGPCRKVCNGIGIGEF |
| | EGFRvIII | KDSLSINATNIKHFKNCTSISG |
| 139 | Amino acid residues 19 to 62 of | ACGADSYEMEEDGVRKCKKCEGPCRKVCNGIGIGEF |
| | EGFRvIII | KDSLSINA |
| 140 | Amino acid residues 19 to 49 of | ACGADSYEMEEDGVRKCKKCEGPCRKVCNGI |
| | EGFRvIII | |
| 141 | Amino acid residues 19 to 45 of | ACGADSYEMEEDGVRKCKKCEGPCRKV |
| | EGFRvIII | |
| 142 | Amino acid residues 19 to 37 of | ACGADSYEMEEDGVRKCKK |
| | EGFRvIII | |
| 143 | Amino acid residues 28 to 45 of | EEDGVRKCKKCEGPCRKV |
| | EGFRvIII | |
| 144 | Amino acid residues 28 to 37 of | EEDGVRKCKK |
| | EGFRvIII | |
| 145 | Amino acid residues 15 to 37 of | ACVRACGADSYEMEEDGVRKCKK |
| | EGFRvIII, with Ser15 to Ala | |
| | mutation | |
| 146 | Amino acid residues 15 to 37 of | SAVRACGADSYEMEEDGVRKCKK |
| | EGFRvIII, with Cys16 to Ala | |
| | mutation | |
| 147 | Amino acid residues 15 to 37 of | SCARACGADSYEMEEDGVRKCKK |
| | EGFRvIII, with Val17 to Ala | |
| | mutation | |
| 148 | Amino acid residues 15 to 37 of | SCVAACGADSYEMEEDGVRKCKK |
| | EGFRvIII, with Arg18 to Ala | |
| | mutation | |
| 149 | Amino acid residues 15 to 37 of | SCVRAAGADSYEMEEDGVRKCKK |
| | EGFRvIII, with Cys20 to Ala | |
| | mutation | |
| 150 | Amino acid residues 15 to 37 of | SCVRACAADSYEMEEDGVRKCKK |
| | EGFRvIII, with Gly21 to Ala | |
| | mutation | |
| 151 | Amino acid residues 15 to 37 of | SCVRACGAASYEMEEDGVRKCKK |
| | EGFRvIII, with Asp23 to Ala | |
| | mutation | |
| 152 | Amino acid residues 15 to 37 of | SCVRACGADAYEMEEDGVRKCKK |
| | EGFRvIII, with Ser24 to Ala | |
| | mutation | |
| 153 | Amino acid residues 15 to 37 of | SCVRACGADSAEMEEDGVRKCKK |
| | EGFRvIII, with Tyr25 to Ala | |
| | mutation | |
| 154 | Amino acid residues 15 to 37 of | SCVRACGADSYAMEEDGVRKCKK |
| | EGFRvIII, with Glu26 to Ala | |
| | mutation | |
| 155 | Amino acid residues 15 to 37 of | SCVRACGADSYEAEEDGVRKCKK |
| | EGFRvIII, with Met27 to Ala | |
| | mutation | |
| 156 | Amino acid residues 15 to 37 of | SCVRACGADSYEMAEDGVRKCKK |
| | EGFRvIII, with Glu28 to Ala | |
| | mutation | |
| 157 | Amino acid residues 15 to 37 of | SCVRACGADSYEMEADGVRKCKK |
| | EGFRvIII, with Glu29 to Ala | |
| | mutation | |
| 158 | Amino acid residues 15 to 37 of | SCVRACGADSYEMEEAGVRKCKK |
| | EGFRvIII, with Asp30 to Ala | |
| | mutation | |
| 159 | Amino acid residues 15 to 37 of | SCVRACGADSYEMEEDAVRKCKK |
| | EGFRvIII, with Gly31 to Ala | |
| | mutation | |
| 160 | Amino acid residues 15 to 37 of | SCVRACGADSYEMEEDGARKCKK |
| | EGFRvIII, with Val32 to Ala | |
| | mutation | |
| 161 | Amino acid residues 15 to 37 of | SCVRACGADSYEMEEDGVAKCKK |
| | EGFRvIII, with Arg33 to Ala | |
| | mutation | |
| 162 | Amino acid residues 15 to 37 of | SCVRACGADSYEMEEDGVRACKK |
| | EGFRvIII, with Lys34 to Ala | |
| | mutation | |
| 163 | Amino acid residues 15 to 37 of | SCVRACGADSYEMEEDGVRKAKK |
| | EGFRvIII, with Cys35 to Ala | |
| | mutation | |
| 164 | Amino acid residues 15 to 37 of | SCVRACGADSYEMEEDGVRKCAK |
| | EGFRvIII, with Lys36 to Ala | |
| | mutation | |
| 165 | Amino acid residues 15 to 37 of | SCVRACGADSYEMEEDGVRKCKA |
| | EGFRvIII, with Lys37 to Ala | |
| | mutation | |
| 166 | Amino acid sequence for single- | EVQLQQSGAELARPGASVKMSCKASGYTFTSYWMH |
| | chain variable fragment composed | WVKQRPGQGLEWIGAIYPGNSDISYNQKFKGKAKLT |
| | of VH, linker, VL sequence | AVTSATTAYMELSSLTNEDSAVYYCTLYDYDPDYWG |
| | derived from 5G6 antibody | QGTTLTVSSGTGGGSGGGGSGGGGSDVVMTQTPLTLS |
| | Linker sequence including | VTIGQPASISCKSSQSLLDSDGKTYLNWLLQRPGQSPK |
| | restriction sites underlined | RLIYLASKLDSGVPDRFTGSGSGTDFTLKISRVEAEDL |
| | but any suitable linker in the | GVYYCWQATHFPWTFGGGTKLEIK |
| | art may be used | |
| 167 | Amino acid sequence for single- | DVQLQESGPGLVKPSQSLSLTCTVTGYSITSDYAWNW |
| | chain variable fragment composed | IRQFPGNKLEWMGYIGYNGRTSYNPSLKSRISITRDTS |
| | of VH, linker, VL sequence | KNQFFLQLNYVTTEDTATFYCARLGRGFAYWGQGTL |
| | derived from 4E11 antibody | VTVSAGTGGGSGGGGSGGGGSDVDILMTQSPSSMSVS |
| | Linker sequence including | LGDTVSITCHASQGINSNIGWLLQKPGKSFKGLIYHGT |
| | restriction sites underlined, | NLEDGVPSRFSGSGSGTDYSLTISSLESEDFADYYCVQ |
| | but any suitable linker in the | YAQFPYTFGGGTKLEIK |
| | art may be used | |
| 168 | Amino Acid exemplary sequence | MLRLLLALNLFPSIQVTGEVQLQQSGAELARPGASVK |
| | for 5G6-CD28-CD3zeta containing | MSCKASGYTFTSYWMHWVKQRPGQGLEWIGAIYPG |
| | chimeric antigen receptor | NSDISYNQKFKGKAKLTAVTSATTAYMELSSLTNEDS |
| | molecule | AVYYCTLYDYDPDYWGQGTTLTVSSGTGGGSGGGGS |
| | Composed of 5G6 scFV, | GGGGSDVVMTQTPLTLSVTIGQPASISCKSSQSLLDSD |
| | a CD8 hinge, human CD28 | GKTYLNWLLQRPGQSPKRLIYLASKLDSGVPDRFTGS |
| | transmembrane domain, human | GSGTDFTLKISRVEAEDLGVYYCWQATHFPWTFGGG |
| | CD28-signaling domain, and | TKLEIKTTTPAPRPPTPAPTIASQPLSLRPEACRPAAGG |
| | human CD3-zeta signaling domain | AVHTRGLDFACDPSKPFWVLVVVGGVLACYSLLVTV |
| | | AFIIFWVRSKRSRLLHSDYMNMTPRRPGPTRKHYQPY |
| | | APPRDFAAYRSASLRVKFSRSADAPAYQQGQNQLYN |
| | | ELNLGRREEYDVLDKRRGRDPEMGGKPQRRKNPQEG |
| | | LYNELQKDKMAEAYSEIGMKGERRRGKGHDGLYQG |
| | | LSTATKDTYDALHMQALPPR |
| 169 | Amino Acid exemplary sequence | MLRLLLALNLFPSIQVTGDVQLQESGPGLVKPSQSLSL |
| | for 4E11-CD28-CD3zeta | TCTVTGYSITSDYAWNWIRQFPGNKLEWMGYIGYNG |
| | containing chimeric antigen | RTSYNPSLKSRISITRDTSKNQFFLQLNYVTTEDTATFY |
| | receptor molecule | CARLGRGFAYWGQGTLVTVSAGTGGGSGGGGSGGG |
| | Composed of 4E11 scFV, a CD8 | GSDVDILMTQSPSSMSVSLGDTVSITCHASQGINSNIG |
| | hinge, human CD28 | WLLQKPGKSFKGLIYHGTNLEDGVPSRFSGSGSGTDY |
| | transmembrane domain, human | SLTISSLESEDFADYYCVQYAQFPYTFGGGTKLEIKTT |
| | CD28-signaling domain, and | TPAPRPPTPAPTIASQPLSLRPEACRPAAGGAVHTRGL |
| | human CD3-zeta signaling domain | DFACDPSKPFWVLVVVGGVLACYSLLVTVAFIIFWVR |
| | | SKRSRLLHSDYMNMTPRRPGPTRKHYQPYAPPRDFAA |
| | | YRSASLRVKFSRSADAPAYQQGQNQLYNELNLGRRE |
| | | EYDVLDKRRGRDPEMGGKPQRRKNPQEGLYNELQKD |
| | | KMAEAYSEIGMKGERRRGKGHDGLYQGLSTATKDTY |
| | | DALHMQALPPR |
| 170 | Amino acid sequence for 4E11 | DVQLQESGPGLVKPSQSLSLTCTVTGYSITSDYAWNW |
| | bi-specific T cell engager | IRQFPGNKLEWMGYIGYNGRTSYNPSLKSRISITRDTS |
| | exemplary sequence | KNQFFLQLNYVTTEDTATFYCARLGRGFAYWGQGTL |
| | Linker sequences underlined, but | VTVSAKTTPPSVYPLAPGSLGTGGGSGGGGSGGGGSD |
| | any suitable linker in the art | VDILMTQSPSSMSVSLGDTVSITCHASQGINSNIGWLL |
| | may be used | QKPGKSFKGLIYHGTNLEDGVPSRFSGSGSGTDYSLTI |
| | CD3-specific scFv engager shown | SSLESEDFADYYCVQYAQFPYTFGGGTKLEIKRADAA |
| | in bold | PTVSIFPPSSKLGDLGGGGSRDDDIKLQQSGAELARP |
| | | GASVKMSCKTSGYTFTRYTMHWVKQRPGQGLEW |
| | | IGYINPSRGYTNYNQKFKDKATLTTDKSSSTAYMQ |
| | | LSSLTSEDSAVYYCARYYDDHYCLDYWGQGTTLTV |
| | | SSVEGGSGGSGGSGGSGGVDDIQLTQSPAIMSASPG |
| | | EKVTMTCRASSSVSYMNWYQQKSGTSPKRWIYDT |
| | | SKVASGVPYRFSGSGSGTSYSLTISSMEAEDAATYY |
| | | CQQWSSNPLTFGAGTKLELK |
| 171 | Exemplary linker | GTGGGGSGGGGSGGGGSDV |
| 172 | Humanized 4E11 light chain | DIQMTQSPSSLSASVGDRVTITCHASQGINSNIGWYQQKP |
| | variable region, variant 1 (hVL1) | GKAPKLLIYHGTNLEDGVPSRFSGSGSGTDYTLTISSLQP |
| | | EDFATYYCVQYAQFPYTFGQGTKLEIK |
| 173 | Humanized 4E11 light chain | DIQMTQSPSSLSASVGDRVTITCHASQGINSNIGWLQQKP |
| | variable region, variant 2 (hVL2) | GKAPKGLIYHGTNLEDGVPSRFSGSGSGTDYTLTISSLQP |
| | | EDFATYYCVQYAQFPYTFGQGTKLEIK |
| 174 | Humanized 4E11 light chain | DIQMTQSPSSLSASVGDRVTITCHASQGINSNIGWLQQKP |
| | variable region, variant 3 (hVL3) | GKAFKGLIYHGTNLEDGVPSRFSGSGSGTDYTLTISSLQP |
| | | EDFATYYCVQYAQFPYTFGQGTKLEIK |
| 175 | Humanized 4E11 heavy chain | QVQLQESGPGLVKPSQTLSLTCTVSGYSITSDYAWNWIR |
| | variable region, variant 1 (hVH1) | QPPGKGLEWIGYIGYNGRTSYNPSLKSRVTISVDTSKNQF |
| | | SLKLSSVTAADTAVYYCARLGRGFAYWGQGTLVTVSS |
| 176 | Humanized 4E11 heavy chain | QVQLQESGPGLVKPSQTLSLTCTVSGYSITSDYAWNWIR |
| | variable region, variant 2 (hVH2) | QPPGKGLEWIGYIGYNGRTSYNPSLKSRVTISRDTSKNQF |
| | | SLKLSSVTAADTAVYYCARLGRGFAYWGQGTLVTVSS |
| 177 | Humanized 4E11 heavy chain | QVQLQESGPGLVKPSQTLSLTCTVSGYSITSDYAWNWIR |
| | variable region, variant 3 (hVH3) | QPPGKGLEWMGYIGYNGRTSYNPSLKSRITISRDTSKNQF |
| | | SLKLSSVTAADTAVYYCARLGRGFAYWGQGTLVTVSS |
| 178 | Light chain human kappa constant | RTVAAPSVFIFPPSDEQLKSGTASVVCLLNNFYPREAKVQ |
| | region | WKVDNALQSGNSQESVTEQDSKDSTYSLSSTLTLSKADY |
| | | EKHKVYACEVTHQGLSSPVTKSFNRGEC |
| 179 | Heavy chain human IgG4 (S228P) | ASTKGPSVFPLAPCSRSTSESTAALGCLVKDYFPEPVTVS |
| | constant region | WNSGALTSGVHTFPAVLQSSGLYSLSSVVTVPSSSLGTKT |
| | | YTCNVDHKPSNTKVDKRVESKYGPPCPPCPAPEFLGGPS |
| | | VFLFPPKPKDTLMISRTPEVTCVVVDVSQEDPEVQFNWY |
| | | VDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLG |
| 180 | Humanized 4E11 light chain, | DIQMTQSPSSLSASVGDRVTITCHASQGINSNIGWYQQKP |
| | variant 1 (hL 1) | GKAPKLLIYHGTNLEDGVPSRFSGSGSGTDYTLTISSLQP |
| | (Variable region underlined) | EDFATYYCVQYAQFPYTFGQGTKLEIKRTVAAPSVFIFPP |
| | | SDEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGEC |
| 181 | Humanized 4E11 light chain, | DIQMTQSPSSLSASVGDRVTITCHASQGINSNIGWLQQKP |
| | variant 2 (hL2) | GKAPKGLIYHGTNLEDGVPSRFSGSGSGTDYTLTISSLQP |
| | (Variable region underlined) | EDFATYYCVQYAQFPYTFGQGTKLEIKRTVAAPSVFIFPP |
| | | SDEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGEC |
| 182 | Humanized 4E11 light chain, | DIQMTQSPSSLSASVGDRVTITCHASQGINSNIGWLQQKP |
| | variant 3 (hL 3) | GKAFKGLIYHGTNLEDGVPSRFSGSGSGTDYTLTISSLQP |
| | (Variable region underlined) | EDFATYYCVQYAQFPYTFGQGTKLEIKRTVAAPSVFIFPP |
| | | SDEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGEC |
| 183 | Humanized 4E11 heavy chain, | QVQLQESGPGLVKPSQTLSLTCTVSGYSITSDYAWNWIR |
| | variant 1 (hH1) | QPPGKGLEWIGYIGYNGRTSYNPSLKSRVTISVDTSKNQF |
| | (Variable region underlined) | SLKLSSVTAADTAVYYCARLGRGFAYWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFLGGPSV |
| | | FLFPPKPKDTLMISRTPEVTCVVVDVSQEDPEVQFNWYV |
| | | DGVEVHNAKTKPREEQFNSTYRVVSVLTVLHQDWLNG |
| | | KEYKCKVSNKGLPSSIEKTISKAKGQPREPQVYTLPPSQE |
| | | EMTKNQVSLTCLVKGFYPSDIAVEWESNGQPENNYKTTP |
| | | PVLDSDGSFFLYSRLTVDKSRWQEGNVFSCSVMHEALH |
| | | NHYTQKSLSLSLG |
| 184 | Humanized 4E11 heavy chain, | QVQLQESGPGLVKPSQTLSLTCTVSGYSITSDYAWNWIR |
| | variant 2 (hH2) | QPPGKGLEWIGYIGYNGRTSYNPSLKSRVTISRDTSKNQF |
| | (Variable region underlined) | SLKLSSVTAADTAVYYCARLGRGFAYWGOGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFLGGPSV |
| | | FLFPPKPKDTLMISRTPEVTCVVVDVSQEDPEVQFNWYV |
| | | DGVEVHNAKTKPREEQFNSTYRVVSVLTVLHQDWLNG |
| | | KEYKCKVSNKGLPSSIEKTISKAKGQPREPQVYTLPPSQE |
| | | EMTKNQVSLTCLVKGFYPSDIAVEWESNGQPENNYKTTP |
| | | PVLDSDGSFFLYSRLTVDKSRWQEGNVFSCSVMHEALH |
| | | NHYTQKSLSLSLG |
| 185 | Humanized 4E11 heavy chain, | QVQLQESGPGLVKPSQTLSLTCTVSGYSITSDYAWNWIR |
| | variant 3 (hH3) | QPPGKGLEWMGYIGYNGRTSYNPSLKSRITISRDTSKNQF |
| | (Variable region underlined) | SLKLSSVTAADTAVYYCARLGRGFAYWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFLGGPSV |
| | | FLFPPKPKDTLMISRTPEVTCVVVDVSQEDPEVQFNWYV |
| | | DGVEVHNAKTKPREEQFNSTYRVVSVLTVLHQDWLNG |
| | | KEYKCKVSNKGLPSSIEKTISKAKGQPREPQVYTLPPSQE |
| | | EMTKNQVSLTCLVKGFYPSDIAVEWESNGQPENNYKTTP |
| | | PVLDSDGSFFLYSRLTVDKSRWQEGNVFSCSVMHEALH |
| | | NHYTQKSLSLSLG |

What is claimed is:

1. A compound comprising the structure of Formula I, or a pharmaceutically acceptable salt thereof:
A-L¹-(L²)ₙ-B Formula I
wherein
A is a chelating moiety or a metal complex thereof,
B is a targeting moiety that is capable of binding to epidermal growth factor receptor variant III (EGFRvIII) or a fragment thereof, wherein the EGFRvIII or fragment thereof comprises a peptide consisting of amino acid residues 1 to 76 of SEQ ID NO: 119;
L¹ is a bond, C═O, C═S, optionally substituted C₁-C₆ alkyl, optionally substituted C₁-C₆ heteroalkyl, optionally substituted aryl, or optionally substituted heteroaryl;
n is an integer between 1 and 5 (inclusive); and
L² each independently has the structure of Formula II:
—X¹-L³-Z¹— Formula II
wherein
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—, or —NR¹—, wherein “*” indicates the attachment point to L³, and R¹ is hydrogen, optionally substituted C₁-C₆ alkyl, optionally substituted C₁-C₆ heteroalkyl, optionally substituted aryl, or optionally substituted heteroaryl;
L³ is optionally substituted C₁-C₅₀ alkyl or optionally substituted C₁-C₅₀ heteroalkyl; and
Z¹ is —CH₂-#, —C(O)-#, —C(S)-#, —OC(O)-#, —C(O)O-#, —NR²C(O)-#, —C(O)NR²-#, or —NR²-#, wherein “#” indicates the attachment point to B, and R² is hydrogen, optionally substituted C₁-C₆ alkyl, optionally substituted C₁-C₆ heteroalkyl, optionally substituted aryl, or optionally substituted heteroaryl.

2. The compound of claim 1, wherein said chelating moiety is selected from the group consisting of DOTA (1,4,7,10-tetraazacyclododecane-1,4,7,10-tetraacetic acid), DOTMA (1R,4R,7R,10R)-α,α′,α″,α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), DO3AM-acetic acid (2-(4,7,10-tris(2-amino-2-oxoethyl)-1,4,7,10-tetraazacyclododecan-1-yl) acetic acid), DOTA-GA anhydride (2,2′,2″-(10-(2,6-dioxotetrahydro-2H-pyran-3-yl)-1,4,7,10-tetraazacyclododecane-1,4,7-triyl)triacetic acid, DOTP (1,4,7,10-tetraazacyclododecane-1,4,7,10-tetra(methylene phosphonic acid)), DOTMP (1,4,6,10-tetraazacyclodecane-1,4,7,10-tetramethylene phosphonic acid, DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaazacyclohexadecane-1,4,7,10,13,16-hexaacetic acid), PEPA (1,4,7,10,13-pentaazacyclopentadecane-N,N′,N″,N′″,N′″-pentaacetic acid), Hoctapa (N,N′-bis(6-carboxy-2-pyridylmethyl)-ethylenediamine-N,N′-diacetic acid), Hadedpa (1,2-[[6-(carboxy)-pyridin-2-yl]-methylamino]ethane), Hophospa (N,N′-(methylenephosphonate)-N,N′-[6-(methoxycarbonyl)pyridin-2-yl]-methyl-1,2-diaminoethane), TTHA (triethylenetetramine-N,N,N′,N″,N′″N″″-hexaacetic acid), DO2P (tetraazacyclododecane dimethanephosphonic acid), HP-DO3A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), octadentate-HOPO (octadentate hydroxypyridinones), and porphyrin.

3. The compound of claim 2, wherein the compound is represented by: 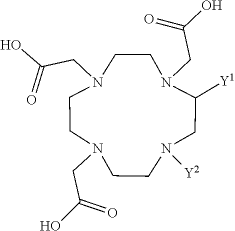
wherein Y¹ is —CH₂OCH₂ (L²)ₙ-B, C═O(L²)ₙ-B, or C═S(L²)ₙ-B and Y² is —CH₂CO₂H; or
wherein Y¹ is H and Y² is L¹-(L²)ₙ-B.

4. The compound of any one of claims 1-3, wherein L¹ is 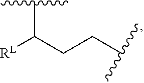
wherein R^{L} is hydrogen or —CO₂H.

5. The compound of any one of claims 1-4,
wherein the metal complex comprises a metal selected from the group consisting of Bi, Pb, Y, Mn, Cr, Fe, Co, Zn, Ni, Tc, In, Ga, Cu, Re, a lanthanide, and an actinide; or
wherein the metal complex comprises a radionuclide selected from the group consisting of ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁸⁹Zr, ⁹⁰Y, ⁹⁷Ru, ⁹⁹Tc, ^{99m}Tc, ¹⁰⁵Rh, ¹⁰⁹Pd, ¹¹¹In, ^{117m}Sn, ¹³³La, ¹³⁴Ce, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ²²⁷Th, and ²²⁹Th.

6. The compound of any one of claims 3-5, wherein Y¹ is H.

7. The compound of any one of claims 1-6 wherein X¹ is —C(O)NR¹—* or —NR¹C(O)—*, “*” indicating the attachment point to L³, and R¹ is H.

8. The compound of any one of claims 1-7, wherein Z¹ is —CH₂—.

9. The compound of any one of claims 1-8, wherein n is 1, and L³ comprises (CH₂CH₂O)₂₋₂₀.

10. The compound of any one of claims 1-8, wherein n is 1, and L³ is (CH₂CH₂O)ₘ(CH₂)_{w}, wherein m and w are each independently an integer between 0 and 10 (inclusive), and at least one of m and w is not 0.

11. The compound of claim 1, wherein A-L-comprises one of the following structures, or a metal complex thereof:

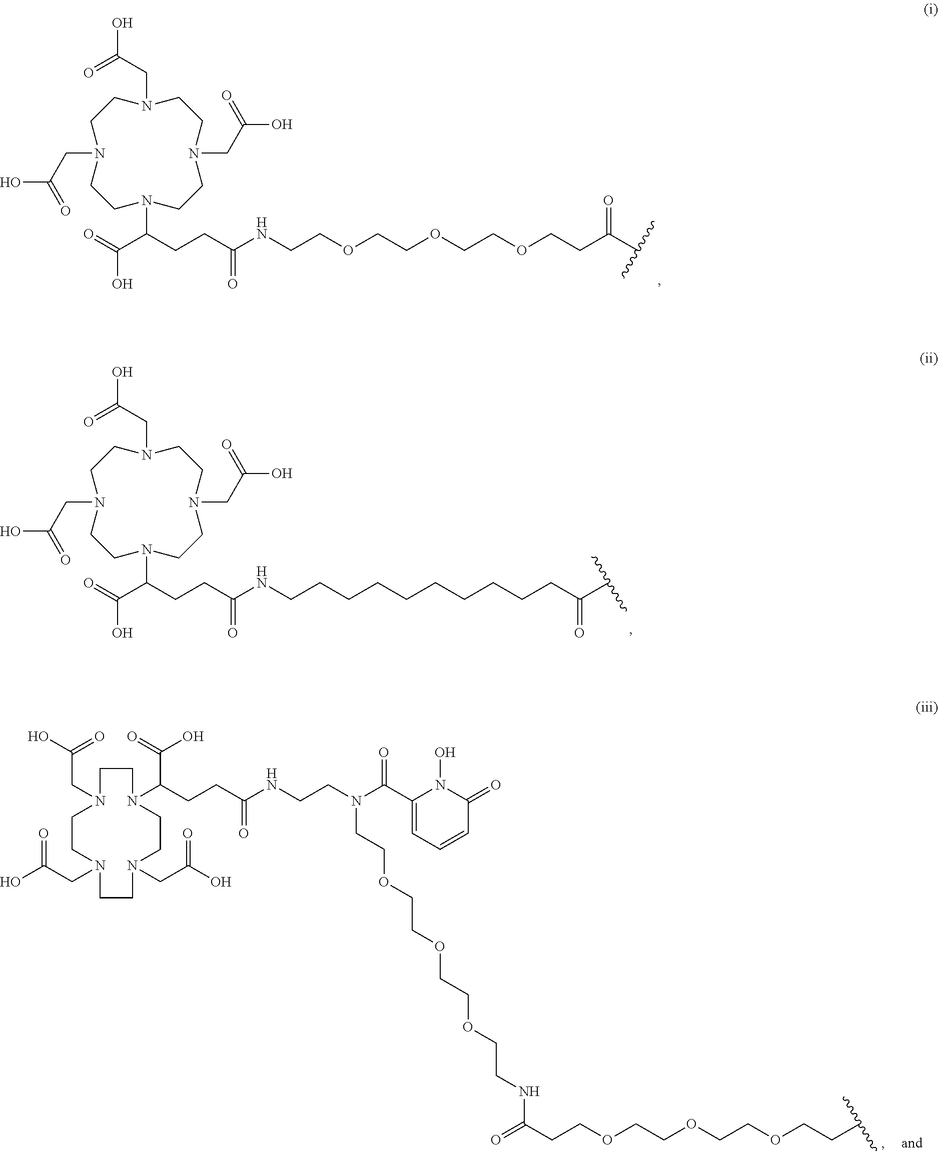 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCNC(=O)CCC(C(=O)O)N1CCN(CC(=O)O)CCN(CC(=O)O)CCN(CC(=O)O)CC1 ; *CCOCCOCCOCCC(=O)NCCOCCOCCOCCN(CCNC(=O)CCC(C(=O)O)N1CCN(CC(=O)O)CCN(CC(=O)O)CCN(CC(=O)O)CC1)C(=O)c1cccc(=O)n1O]

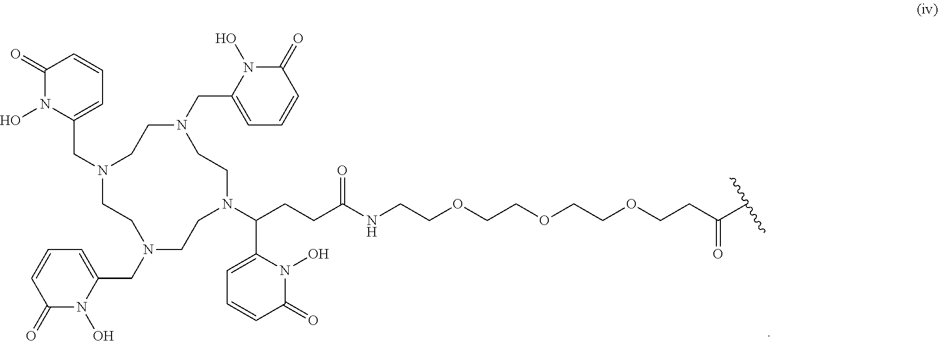 [SMILES: *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

12. The compound of claim 1, wherein the radioimmunoconjugate comprises the following structure, or a metal complex thereof:

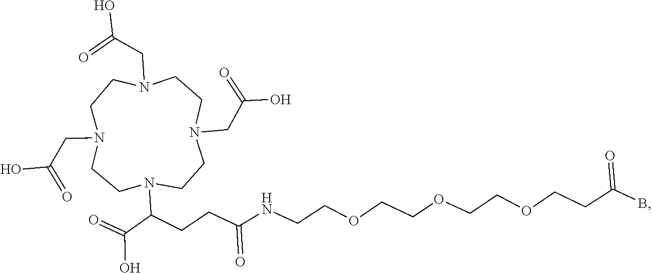

13. The compound of any one of claims 1-12, wherein A is a metal complex of a chelating moiety, and the metal complex comprises a radionuclide.

14. The compound of claim 13, wherein the radionuclide is ⁶⁸Ga, ¹¹¹In, ¹⁷⁷Lu, or ²²⁵Ac.

15. The compound of claim 13, wherein the radionuclide is ²²⁵Ac.

16. The compound of claim 13, wherein the radionuclide is an alpha emitter selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb), or a progeny thereof.

17. The compound of claim 16, wherein the alpha emitter is ²²⁵Ac or a progeny thereof.

18. The compound of any one of claims 1-17, wherein the targeting moiety comprises an antibody or antigen-binding fragment thereof.

19. The compound of claim 18, wherein said antibody or antigen-binding fragment thereof comprises:
a. a light chain variable region comprising a CDRL1 having the amino acid sequence of SEQ ID NO:8, a CDRL2 sequence of SEQ ID NO:9 and a CDRL3 sequence of SEQ ID NO: 10 and a heavy chain variable region comprising a CDRH1 sequence of SEQ ID NO: 13, a CDRH2 sequence of SEQ ID NO:14 and a CDRH3 sequence of SEQ ID NO: 15;
b. a light chain variable region comprising a CDRL1 having the amino acid sequence set forth in SEQ ID NO: 18, a CDRL2 having the amino acid sequence set forth in SEQ ID NO:19 and a CDRL3 having the amino acid sequence set forth in SEQ ID NO: 20 and a heavy chain variable region comprising a CDRH1 having the amino acid sequence set forth in SEQ ID NO:23, a CDRH2 having the amino acid sequence set forth in SEQ ID NO:24 and a CDRH3 having the amino acid sequence set forth in SEQ ID NO:25;
c. a light chain variable region comprising a CDRL1 having the amino acid sequence set forth in SEQ ID NO:28, a CDRL2 having the amino acid sequence set forth in SEQ ID NO:29 and a CDRL3 having the amino acid sequence set forth in SEQ ID NO: 30 and a heavy chain variable region comprising a CDRH1 having the amino acid sequence set forth in SEQ ID NO:33, a CDRH2 having the amino acid sequence set forth in SEQ ID NO:34 and a CDRH3 having the amino acid sequence set forth in SEQ ID NO:35;
d. a light chain variable region comprising a CDRL1 having the amino acid sequence set forth in SEQ ID NO:38, a CDRL2 having the amino acid sequence set forth in SEQ ID NO:39 and a CDRL3 having the amino acid sequence set forth in SEQ ID NO: 40 and a heavy chain variable region comprising a CDRH1 having the amino acid sequence set forth in SEQ ID NO:43, a CDRH2 having the amino acid sequence set forth in SEQ ID NO:44 and a CDRH3 having the amino acid sequence set forth in SEQ ID NO:45;
e. a light chain variable region comprising a CDRL1 having the amino acid sequence set forth in SEQ ID NO:48, a CDRL2 having the amino acid sequence set forth in SEQ ID NO:49 and a CDRL3 having the amino acid sequence set forth in SEQ ID NO: 50 and a heavy chain variable region comprising a CDRH1 having the amino acid sequence set forth in SEQ ID NO:53, a CDRH2 having the amino acid sequence set forth in SEQ ID NO:54 and a CDRH3 having the amino acid sequence set forth in SEQ ID NO:55;
f. a light chain variable region comprising a CDRL1 having the amino acid sequence set forth in SEQ ID NO:58, a CDRL2 having the amino acid sequence set forth in SEQ ID NO:59 and a CDRL3 having the amino acid sequence set forth in SEQ ID NO: 60 and a heavy chain variable region comprising a CDRH1 having the amino acid sequence set forth in SEQ ID NO:63, a CDRH2 having the amino acid sequence set forth in SEQ ID NO:64 and a CDRH3 having the amino acid sequence set forth in SEQ ID NO:65;
g. a light chain variable region comprising a CDRL1 having the amino acid sequence set forth in SEQ ID NO:68, a CDRL2 having the amino acid sequence set forth in SEQ ID NO:69 and a CDRL3 having the amino acid sequence set forth in SEQ ID NO: 70 and a heavy chain variable region comprising a CDRH1 having the amino acid sequence set forth in SEQ ID NO:78, a CDRH2 having the amino acid sequence set forth in SEQ ID NO: 79 and a CDRH3 having the amino acid sequence set forth in SEQ ID NO:80; or
h. a light chain variable region comprising a CDRL1 having the amino acid sequence set forth in SEQ ID NO: 73, a CDRL2 having the amino acid sequence set forth in SEQ ID NO:74 and a CDRL3 having the amino acid sequence set forth in SEQ ID NO: 75 and a heavy chain variable region comprising a CDRH1 having the amino acid sequence set forth in SEQ ID NO:78, a CDRH2 having the amino acid sequence set forth in SEQ ID NO:79 and a CDRH3 having the amino acid sequence set forth in SEQ ID NO:80.

20. The compound of claim 18, wherein said antibody or antigen-binding fragment thereof comprises:
a. a light chain variable region comprising an amino acid sequence at least 80% identical to the amino acid sequence set forth in SEQ ID NO: 118 or substantially identical to SEQ ID NO:118 and a heavy chain variable region comprising an amino acid sequence at least 80% identical to the amino acid sequence set forth in SEQ ID NO: 116 or substantially identical to SEQ ID NO:116;
b. a light chain variable region comprising an amino acid sequence at least 80% identical to the amino acid sequence set forth in SEQ ID NO: 115 or substantially identical to SEQ ID NO:115 and a heavy chain variable region comprising an amino acid sequence at least 80% identical to the amino acid sequence set forth in SEQ ID NO: 116 or substantially identical to SEQ ID NO:116; or
c. a light chain variable region comprising an amino acid sequence at least 80% identical to the amino acid sequence set forth in SEQ ID NO: 118 or substantially identical to SEQ ID NO: 118 and a heavy chain variable region comprising an amino acid sequence at least 80% identical to the amino acid sequence set forth in SEQ ID NO: 62 or substantially identical to SEQ ID NO:62.

21. The compound of claim 18, wherein said antibody or antigen-binding fragment thereof comprises:
a. a light chain variable region comprising a sequence at least 80% identical to the amino acid sequence set forth in SEQ ID NO:7 or substantially identical to SEQ ID NO: 7 and a heavy chain variable region comprising a sequence at least 80% identical to the amino acid sequence set forth in SEQ ID NO: 12 or substantially identical to SEQ ID NO:12;
b. a light chain variable region comprising an amino acid sequence at least 80% identical to the amino acid sequence set forth in SEQ ID NO: 17 or substantially identical to SEQ ID NO: 17 and a heavy chain variable region comprising an amino acid sequence at least 80% identical to the amino acid sequence set forth in SEQ ID NO: 22 or substantially identical to SEQ ID NO:22;
c. a light chain variable region comprising an amino acid sequence at least 80% identical to the amino acid sequence set forth in SEQ ID NO:27 or substantially identical to SEQ ID NO:27 and a heavy chain variable region comprising an amino acid sequence at least 80% identical to the amino acid sequence set forth in SEQ ID NO: 32 or substantially identical to SEQ ID NO:32;
d. a light chain variable region comprising an amino acid sequence at least 80% identical to the amino acid sequence set forth in SEQ ID NO: 37 or substantially identical to SEQ ID NO:37 and a heavy chain variable region comprising an amino acid sequence at least 80% identical to the amino acid sequence set forth in SEQ ID NO: 42 or substantially identical to SEQ ID NO:42;
e. a light chain variable region comprising an amino acid sequence at least 80% identical to the amino acid sequence set forth in SEQ ID NO:47 or substantially identical to SEQ ID NO:47 and a heavy chain variable region comprising an amino acid sequence at least 80% identical to the amino acid sequence set forth in SEQ ID NO: 52 or substantially identical to SEQ ID NO:52;
f. a light chain variable region comprising an amino acid sequence at least 80% identical to the amino acid sequence set forth in SEQ ID NO:57 or substantially identical to SEQ ID NO:57 and a heavy chain variable region comprising an amino acid sequence at least 80% identical to the amino acid sequence set forth in SEQ ID NO: 62 or substantially identical to SEQ ID NO:62;
g. a light chain variable region comprising an amino acid sequence at least 80% identical to the amino acid sequence set forth in SEQ ID NO:67 or substantially identical to SEQ ID NO:67 and a heavy chain variable region comprising an amino acid sequence at least 80% identical to the amino acid sequence set forth in SEQ ID NO: 77 or substantially identical to SEQ ID NO:77, the amino acid set forth in SEQ ID NO: 92 or substantially identical to SEQ ID NO:92 or the amino acid sequence set forth in SEQ ID NO: 102 or substantially identical to SEQ ID NO:102; or
h. a light chain variable region comprising an amino acid sequence at least 80% identical to the amino acid sequence set forth in SEQ ID NO:72 or substantially identical to SEQ ID NO: 72 and a heavy chain variable region comprising an amino acid sequence at least 80% identical to the amino acid sequence set forth in SEQ ID NO: 77 or substantially identical to SEQ ID NO:77 or the amino acid set forth in SEQ ID NO: 92 or substantially identical to SEQ ID NO:92.

22. The compound claim 18, wherein said antibody or antigen-binding fragment thereof comprises:
a. a light chain comprising an amino acid sequence at least 80% identical to the amino acid sequence set forth in SEQ ID NO:108 or substantially identical to SEQ ID NO: 108 and a heavy chain comprising an amino acid sequence at least 80% identical to the amino acid sequence set forth in SEQ ID NO: 107 or substantially identical to SEQ ID NO: 107; or
b. a light chain comprising an amino acid sequence at least 80% identical to the amino acid sequence set forth in SEQ ID NO:110 or substantially identical to SEQ ID NO: 110 and a heavy chain comprising an amino acid sequence at least 80% identical to the amino acid sequence set forth in SEQ ID NO: 109 or substantially identical to SEQ ID NO: 109.

23. The compound of claim 19, wherein said antibody or antigen-binding fragment thereof comprises a light chain variable region comprising a CDRL1 having the amino acid sequence set forth in SEQ ID NO:38, a CDRL2 having the amino acid sequence set forth in SEQ ID NO: 39 and a CDRL3 having the amino acid sequence set forth in SEQ ID NO:40 and a heavy chain variable region comprising a CDRH1 having the amino acid sequence set forth in SEQ ID NO: 43, a CDRH2 having the amino acid sequence set forth in SEQ ID NO:44 and a CDRH3 having the amino acid sequence set forth in SEQ ID NO:45.

24. The compound of claim 23, wherein said antibody or antigen-binding fragment thereof comprises:
a. a light chain variable region having the amino acid sequence set forth in SEQ ID NO: 172 and a heavy chain variable region having the amino acid sequence set forth in SEQ ID NO: 175;
b. a light chain variable region having the amino acid sequence set forth in SEQ ID NO: 173 and a heavy chain variable region having the amino acid sequence set forth in SEQ ID NO: 175;
c. a light chain variable region having the amino acid sequence set forth in SEQ ID NO: 174 and a heavy chain variable region having the amino acid sequence set forth in SEQ ID NO: 175;
d. a light chain variable region having the amino acid sequence set forth in SEQ ID NO: 172 and a heavy chain variable region having the amino acid sequence set forth in SEQ ID NO: 176;
e. a light chain variable region having the amino acid sequence set forth in SEQ ID NO: 173 and a heavy chain variable region having the amino acid sequence set forth in SEQ ID NO: 176;
f. a light chain variable region having the amino acid sequence set forth in SEQ ID NO: 174 and a heavy chain variable region having the amino acid sequence set forth in SEQ ID NO: 176;
g. a light chain variable region having the amino acid sequence set forth in SEQ ID NO: 172 and a heavy chain variable region having the amino acid sequence set forth in SEQ ID NO: 177;
h. a light chain variable region having the amino acid sequence set forth in SEQ ID NO: 173 and a heavy chain variable region having the amino acid sequence set forth in SEQ ID NO: 177; or
i. a light chain variable region having the amino acid sequence set forth in SEQ ID NO: 174 and a heavy chain variable region having the amino acid sequence set forth in SEQ ID NO: 177.

25. The compound of claim 24, wherein said antibody or antigen-binding fragment thereof comprises:
a. a light chain variable region having the amino acid sequence set forth in SEQ ID NO: 174 and a heavy chain variable region having the amino acid sequence set forth in SEQ ID NO: 176; or
b. a light chain variable region having the amino acid sequence set forth in SEQ ID NO: 172 and a heavy chain variable region having the amino acid sequence set forth in SEQ ID NO: 177.

26. The compound of claim 23, 24, or 25, wherein said antibody, or antigen-binding fragment thereof comprises:
a. a light chain region having the amino acid sequence set forth in SEQ ID NO: 180 and a heavy chain region having the amino acid sequence set forth in SEQ ID NO: 183;
b. a light chain region having the amino acid sequence set forth in SEQ ID NO: 181 and a heavy chain region having the amino acid sequence set forth in SEQ ID NO: 183;
c. a light chain region having the amino acid sequence set forth in SEQ ID NO: 182 and a heavy chain region having the amino acid sequence set forth in SEQ ID NO: 183;
d. a light chain region having the amino acid sequence set forth in SEQ ID NO: 180 and a heavy chain region having the amino acid sequence set forth in SEQ ID NO: 184;
e. a light chain region having the amino acid sequence set forth in SEQ ID NO: 181 and a heavy chain region having the amino acid sequence set forth in SEQ ID NO: 184;
f. a light chain region having the amino acid sequence set forth in SEQ ID NO: 182 and a heavy chain region having the amino acid sequence set forth in SEQ ID NO: 184;
g. a light chain region having the amino acid sequence set forth in SEQ ID NO: 180 and a heavy chain region having the amino acid sequence set forth in SEQ ID NO: 185;
h. a light chain region having the amino acid sequence set forth in SEQ ID NO: 181 and a heavy chain region having the amino acid sequence set forth in SEQ ID NO: 185; or
i. a light chain region having the amino acid sequence set forth in SEQ ID NO: 182 and a heavy chain region having the amino acid sequence set forth in SEQ ID NO: 185.

27. The compound of any one of claims 18-26, wherein the antibody or antigen-binding fragment thereof is a monoclonal antibody, a polyclonal antibody, a humanized antibody, a chimeric antibody, a human antibody, a single chain antibody, a multispecific antibody, or an antigen-binding fragment thereof.

28. The compound of any one of claims 18-27, wherein the antibody is a humanized monoclonal antibody.

29. The compound of any one of claims 1-25, 27, and 28, wherein said antibody or antigen-binding fragment thereof comprises a human IgG1 constant region.

30. The compound of any one of claims 1-21, 23-25, 27, and 28, wherein said antibody or antigen-binding fragment thereof comprises a human IgG2 constant region.

31. The compound of any one of claims 1-22, and 24-28, wherein said antibody or antigen-binding fragment thereof comprises a human IgG4 constant region.

32. The compound of claim 26, wherein said antibody or antigen-binding fragment thereof comprises a human IgG4 (S228P) constant region.

33. The compound of any one of claims 1-32, wherein the antigen-binding fragment comprises a scFv, a Fab, a Fab′ or a (Fab′)₂.

34. The compound of claim 1, wherein the compound comprises the following structure: 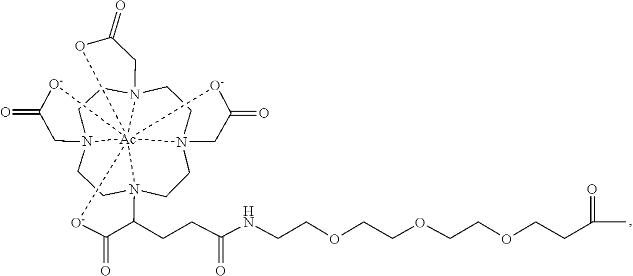
wherein · is an antibody or an antigen-binding fragment thereof that is capable of binding to EGFRvIII or a fragment thereof.

35. The compound of claim 34, wherein the antibody or an antigen-binding fragment thereof is linked to A-L-via the side-chain amino group of a lysine residue.

36. A pharmaceutical composition comprising a compound of any one of claims 1-35 and a pharmaceutically acceptable carrier, diluent, or excipient.

37. A method of radiation treatment planning and/or radiation treatment of cancer, the method comprising administering to a subject in need thereof a compound of any one of claims 1-35 or the pharmaceutical composition of claim 36.

38. A method of treating or preventing cancer that comprises cells expressing EGFRvIII, the method comprising administering to a subject in need thereof a compound of any one of claims 1-35 or the pharmaceutical composition of claim 36 in a therapeutically effective amount.

39. A method of treating or preventing cancer that comprises cells expressing EGFRvIII, the method comprising administering to a subject in need thereof a first dose of a compound of any one of claims 1-35 or a pharmaceutical composition of claim 36 in an amount effective for radiation treatment planning, followed by administering subsequent doses of a compound of any one of claims 1-35 or the pharmaceutical composition of claim 36 in a therapeutically effective amount.

40. The method of claim 39, wherein the compound or pharmaceutical composition administered in the first dose and the compound or pharmaceutical composition administered in a subsequent dose are the same.

41. The method of claim 39, wherein the compound or pharmaceutical composition administered in the first dose and the compound or pharmaceutical composition administered in a subsequent dose are different.

42. The method of any one of claims 37-41, wherein the cancer is glioblastoma multiforme or carcinoma.

43. The method of any one of claims 37-42, further comprising administering an antiproliferative agent, a radiation sensitizer, or an immunomodulatory agent.

44. The method of claim 43, comprising administering a compound of any one of claims 1-35 or the pharmaceutical composition of claim 36 in combination with an antiproliferative agent, in the absence or presence of external beam radiation.

45. The method of claim 44, wherein the antiproliferative agent is temozolomide (TMZ).

46. The method of any one of claims 38-45, wherein the therapeutically effective amount of the compound of any one of claims 1-35 or the pharmaceutical composition of claim 36 is administered in multiple doses.

47. The method of any one of claims 38-45, wherein the therapeutically effective amount of the compound of any one of claims 1-35 or the pharmaceutical composition of claim 36 is administered in a single dose.
